(19) United States
(12) Patent Application Publication
(10) Pub. No.: US 2026/0207638 A1
(43) Pub. Date: Jul. 23, 2026

(54) ORAL TABLET FORMULATIONS

(71) Applicant: Gilead Sciences, Inc., Foster City, CA (US)

(72) Inventors: Melissa A. King, San Mateo, CA (US); Sahar Rahmani, Fremont, CA (US); Morteza Sarmadi, Mountain View, CA (US); Joshua C. Savage, San Mateo, CA (US)

(21) Appl. No.: 19/413,193

(22) Filed: Dec. 9, 2025

Related U.S. Application Data
(63) Continuation of application No. 19/250,335, filed on Jun. 26, 2025, now abandoned.
(60) Provisional application No. 63/665,227, filed on Jun. 27, 2024.

(51) Int. Cl. A61K 31/675; A61K 9/20; A61P 31/18

(52) U.S. Cl. CPC A61K 31/675; A61K 9/2018; A61K 9/2027; A61K 9/2054; A61P 31/18

(57) ABSTRACT

The present disclosure relates to pharmaceutical formulations comprising a HIV capsid inhibitor and methods for the treatment or prevention of a human immunodeficiency virus (HIV) infection in a patient.

### CROSS REFERENCE TO RELATED APPLICATIONS

This application is a continuation of U.S. Ser. No. 19/250,335, filed Jun. 26, 2025, which claims the benefit of U.S. Provisional Application 63/665,227, filed on Jun. 27, 2024, the entire content of which are hereby incorporated by reference in its entirety.

### TECHNICAL FIELD

The present disclosure relates to pharmaceutical formulations comprising a HIV capsid inhibitor and methods for the treatment or prevention of a human immunodeficiency virus (HIV) infection in a patient.

### BACKGROUND

The viral capsid protein (CA) is essential for multiple stages of the HIV life cycle. During viral maturation following the processing of Gag polyprotein by the HIV protease, CA self-assembles into the conical shaped core characteristic of mature HIV-1 virions. Contained within this capsid core are the viral RNA, nucleocapsid, reverse transcriptase, and integrase. Failure to generate a suitable core precludes infectivity. In addition, CA contributes to multiple essential processes during the early stages of HIV replication, including important roles in regulating proper capsid core disassembly (uncoating) kinetics to ensure efficient and productive viral DNA synthesis via coupled reverse transcription, and contributes to the active transport of pre-integration complexes into the nuclear compartment to support viral DNA integration into transcriptionally active loci. Defects in the proper function of capsid ultimately inhibit efficient nuclear uptake and integration of viral DNA into the host genome.

Human immunodeficiency virus type 1 infection is a life-threatening and serious disease of major public health significance, with approximately 38 million people infected worldwide and approximately 26 million on antiretroviral (ARV) treatment (UNAIDS. Global HIV & AIDS statistics, 2020 fact sheet). Advances in combination ARV therapy for HIV have led to significant improvements in morbidity and mortality by suppressing viral replication, preserving immunologic function, and averting disease progression to AIDS.

### SUMMARY

The present disclosure provides, inter alia, a tablet comprising a compound of Formula Ia:

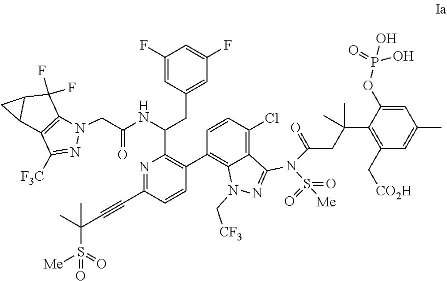 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, and one or more pharmaceutically acceptable excipients.

The present disclosure further provides a method of treating or preventing human immunodeficiency virus (HIV) infection in a patient, comprising administering to the patient a tablet provided herein.

The present disclosure further provides a tablet provided herein, for use in any of the methods described herein.

The present disclosure further provides use of a tablet provided herein, for use in any of the methods described herein.

The present disclosure further provides use of a tablet provided herein, for use in preparation of a medicament for use in any of the methods described herein.

### DESCRIPTION OF DRAWINGS

FIG. 1 shows the structure of 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl)but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid (i.e., Compound 1) and its restricted rotational axes.

FIG. 2 shows a manufacturing process flow diagram for preparing the tablets described in Example 1.

FIGS. 3A-3B show a plots of the plasma concentration of lenacapavir (LEN) in dogs as a function of time upon administration of a non-precipitating solution comprising Compound 1 (55 mg fixed dose); or a dry granulated tablet comprising Compound 1 (100 mg or 55 mg fixed dose).

FIG. 4 shows a representative XRPD pattern of Compound 1, crystalline Form I.

FIG. 5 shows a representative DSC thermogram of Compound 1, crystalline Form I.

FIG. 6 shows a representative TGA thermogram of Compound 1, crystalline Form I.

FIG. 7 shows a representative DVS analysis of Compound 1, crystalline Form I.

FIG. 8 shows mean and standard deviation (SD) of lenacapavir (LEN) plasma concentration-time profiles in a pharmacokinetic (PK) analysis set of single ascending dose (SAD) cohorts. BLQ=below the limit of quantitation; LLOQ=lower limit of quantitation; Values BLQ were treated as 0 for predose and postdose time points for summary purposes. If more than one-third of postdose values were BLQ, then the mean and SD were not presented at that time point. Lower error bars ≤0 are not presented at that time point. Horizontal dashed line indicates LLOQ, defined as 0.5 ng/mL for LEN.

FIG. 9 shows mean and SD of LEN plasma concentration-time profiles in a PK analysis set of multiple ascending dose (MAD) cohorts. Values BLQ were treated as 0 for predose and postdose time points for summary purposes. If more than one-third of postdose values are BLQ, then the mean and SD were not presented at that time point. Lower error bars ≤0 are not presented at that time point. Horizontal dashed line indicates LLOQ, defined as 0.5 ng/mL for LEN.

FIG. 10 shows mean and SD LEN plasma concentration-time profiles in a PK analysis set of food effect (FE) Cohort 12 vs SAD Cohort 5. Values BLQ were treated as 0 for predose and postdose time points for summary purposes. If more than one-third of postdose values are BLQ, then the mean and SD were not presented at that time point. Lower error bars ≤0 are not presented at that time point. Horizontal dashed line indicates LLOQ, defined as 0.5 ng/mL for LEN.

FIG. 11 shows mean and SD LEN plasma concentration-time profiles in a PK analysis set of FE Cohort 11 vs MAD Cohort 7 Week 1. Values BLQ were treated as 0 for predose and postdose time points for summary purposes. If more than one third of postdose values are BLQ, then the mean and SD were not presented at that time point. Lower error bars ≤0 are not presented at that time point. Horizontal dashed line indicates LLOQ, defined as 0.5 ng/mL for LEN.

FIG. 12 shows a manufacturing process flow diagram for preparing the tablets described in Example 14.

FIGS. 13A-13C show dissolution profiles of Compound 1 100 mg (FIG. 13A), 300 mg (FIG. 13B), and 400 mg (FIG. 13C) tablets.

FIG. 14 shows the dissolution profile of Compound 1, 200 mg tablets containing 27.25 w/w % mannitol and 27.25 w/w % microcrystalline cellulose.

### DETAILED DESCRIPTION

Pre-exposure prophylaxis (PrEP) strategies have been used to prevent transmission of HIV-1 infection. In locations where uptake of PrEP is high, there has been a significant population-level reduction in HIV incidence, demonstrating the potential for PrEP to contribute to population-wide HIV control (see e.g., Grulich et al, The Lancet. HIV, 2018; 5(11):e629-e37; and Sullivan et al, Abstract LBPEC036, International AIDS Conference (IAC); July 2018, 23-27). However, for many individuals at risk for HIV, the uptake of daily oral PrEP has been a challenge. In the US, it is estimated that 1.1 million persons are at risk for HIV, yet only 240,000 are on Descovy or Truvada, and another 400,000 more started but have stopped daily oral PrEP. Several reasons likely contribute to the suboptimal uptake of PrEP, including lack of health system accessibility, medical mistrust due to experiences of homophobia, transphobia, and stigma, lack of willingness to take daily oral PrEP, concerns about side effects, and low self-perception of risk (see e.g., Center for Disease Control and Prevention, HIV/AIDS: Pre-exposure prophylaxis (PrEP); cdc.gov/hiv/basics/prep.html, 2017; and Wood et al, AIDS Behav. 2019; 23 (10):2719-29).

Lenacapavir (LEN), a human immunodeficiency virus type 1 (HIV-1) capsid inhibitor, has the potential to provide an additional option for populations at risk of HIV acquisition. Lenacapavir can help address substantial existing PrEP barriers including 1) requirement for daily adherence, 2) stigma and concerns about disclosure and discrimination or other social harms, 3) oral medication-associated adverse events (AEs), including gastrointestinal tolerability, and 4) challenges with access to health care providers in overburdened health systems or in geographical PrEP deserts. Thus, LEN has the potential to increase the uptake of, adherence to, and thereby the scalability of PrEP, thus contributing to the overarching goal of ending the HIV-1 epidemic.

The present invention relates to pharmaceutical compositions (e.g., tablets) comprising 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl)but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid (i.e., Compound 1, structure shown below; see e.g. U.S. Pat. No. 11,787,825, the disclosure of which is incorporated herein by reference in its entirety).

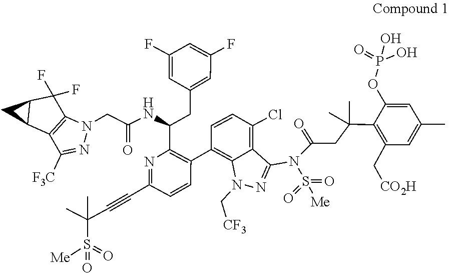 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

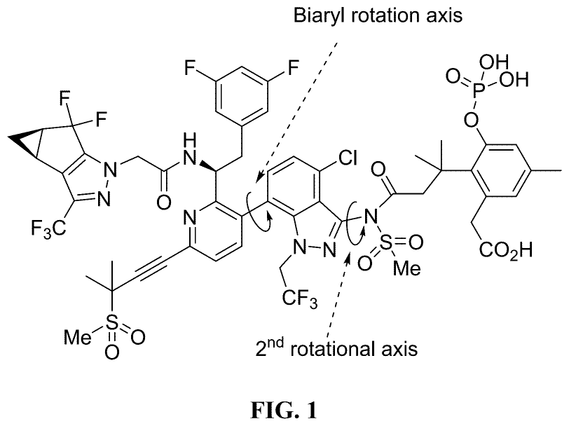

Compound 1 has two restricted rotational axes, resulting in 4 atropisomers (see FIG. 1) that may be detected by ¹⁹F-NMR. In deuterated DMSO at 25° C., the half-life of conversion from the major to the minor atropisomer for the biaryl rotation is about 71.6 hours with equilibrium ratio at about 3:1, and the half-life of interconversion at the 2^{nd} rotational axis is about 7 minutes with equilibrium ratio at about 4:3.

Compound 1 converts to lenacapavir (i.e., N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide), an HIV capsid inhibitor that is in development as a long-acting treatment for HIV, in the gastrointestinal tract when administered to a subject (e.g., a human patient).

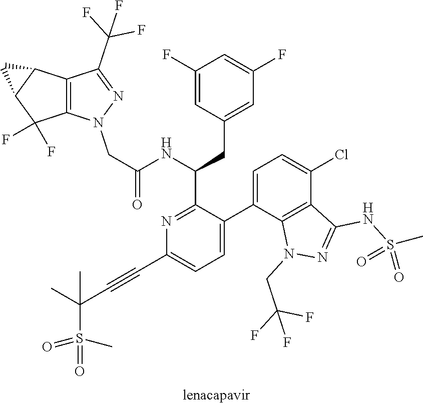 [SMILES: CC(C)(C#Cc1ccc(-c2ccc(Cl)c3c(NS(C)(=O)=O)nn(CC(F)(F)F)c23)c([C@H](Cc2cc(F)cc(F)c2)NC(=O)Cn2nc(C(F)(F)F)c3c2C(F)(F)[C@@H]2C[C@H]32)n1)S(C)(=O)=O]

Synthesis and characterization of lenacapavir, and salts thereof, are described, for example, in US 20180051005 and US 20190300505, the contents of each of which are hereby incorporated by reference in their entireties. Various forms and/or uses of the compounds of lenacapavir are disclosed, for example, in US 20190083478, US 20190084963, US 20200038389A1, and US 20210188815, the contents of each of which are hereby incorporated by reference in their entireties.

As used herein and in the appended claims, the singular forms “a” and “an”, and “the” include plural referents unless the context clearly dictates otherwise. Thus, e.g., reference to “the compound” includes a plurality of such compounds and reference to “the assay” includes reference to one or more assays, and so forth.

Reference to “about” a value or parameter herein includes (and describes) embodiments that are directed to that value or parameter per se. In certain embodiments, the term “about” may include the indicated amount ±10%. In other embodiments, the term “about” may include the indicated amount ±5%. In certain other embodiments, the term “about” may include the indicated amount ±1%. Also, the term “about X” includes description of “X”.

The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds and salts described herein contain one or more asymmetric centers and/or hindered rotation about a bond axis and may thus give rise to enantiomers, diastereomers, and other stereoisomeric forms that may be defined, in terms of absolute stereochemistry, as (R)- or (S)-. The present disclosure is meant to include all such possible isomers, including racemic mixtures, scalemic mixtures, diastereomeric mixtures, optically pure forms and intermediate mixtures. Optically active (R)- and (S)-isomers may be prepared using chiral synthons or chiral reagents, or resolved using conventional techniques.

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s).

One skilled in the art understands that a compound structure may be named or identified using commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC). Accordingly, the compound structure for Compound 1 provided above may also be named or identified as 2-(2-(4-(N-(4-chloro-7-(2-((S)-1-(2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamido)-2-(3,5-difluorophenyl)ethyl)-6-(3-methyl-3-(methylsulfonyl)but-1-yn-1-yl)pyridin-3-yl)-1-(2,2,2-trifluoroethyl)-1H-indazol-3-yl)methylsulfonamido)-2-methyl-4-oxobutan-2-yl)-5-methyl-3-(phosphonooxy)phenyl)acetic acid.

### Pharmaceutical Compositions

Accordingly, the present disclosure provides pharmaceutical compositions comprising a compound of Formula Ia or Tb:

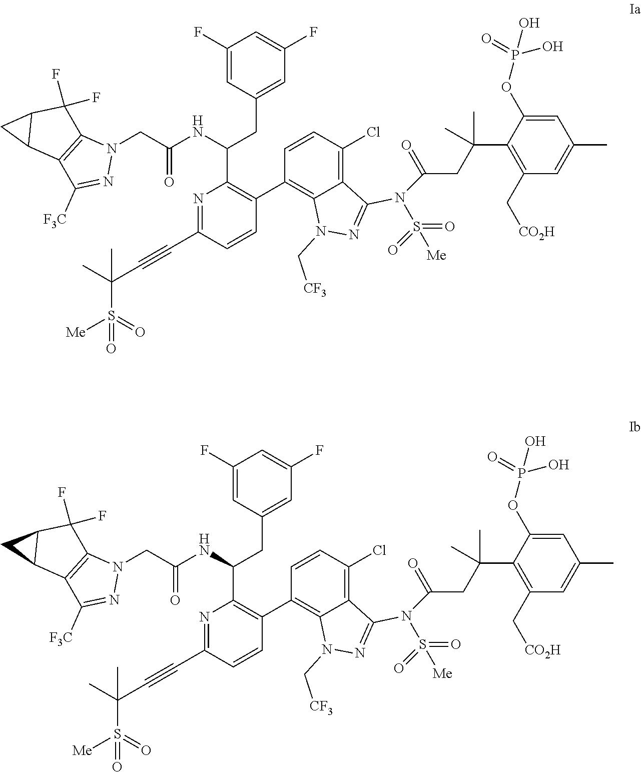 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1 ; Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, and at least one pharmaceutically acceptable carrier.

Pharmaceutical compositions of the disclosure are formulated so as to allow the active ingredients contained therein to be bioavailable upon administration of the composition to a patient. Compositions that will be administered to a patient take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of the compound of Formula Ia or Ib, or a pharmaceutically acceptable salt thereof, for treating an HIV infection, prevention of an HIV infection, or reducing the risk of acquiring an HIV infection, as described herein.

The term “therapeutically effective amount” or “effective amount” of a solid form described herein means an amount sufficient to effect treatment when administered to a subject, to provide a therapeutic benefit such as amelioration of symptoms or slowing of disease progression. For example, a therapeutically effective amount may be an amount sufficient to improve a symptom of a Retroviridae viral infection, including but not limited to HIV infection. The therapeutically effective amount may vary depending on the subject, and the disease or condition being treated, the weight and age of the subject, the severity of the disease or condition, and the manner of administering, which can readily be determined by one of ordinary skill in the art.

As used herein, “pharmaceutically acceptable carrier” is meant to refer to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, the compound of Formula Ia or Ib is administered as a salt, such as a pharmaceutically acceptable salt. A salt generally refers to a derivative of a disclosed compound wherein the parent compound is modified by converting an existing acid or base moiety to its salt form. A pharmaceutically acceptable salt is one that, within the scope of sound medical judgment, is suitable for use in contact with the tissues of human beings and animals without excessive toxicity, irritation, allergic response, or other problem or complication, commensurate with a reasonable benefit/risk ratio. Examples of pharmaceutically acceptable salts include, but are not limited to, mineral or organic acid salts of basic residues such as amines, alkali or organic salts of acidic residues such as carboxylic acids, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

In some embodiments, the pharmaceutical composition provided herein is a tablet. In some embodiments, the tablet provided herein comprises a compound of Formula Ia or Ib, or a pharmaceutically acceptable salt thereof, and one or more pharmaceutically acceptable excipients. In some embodiments, the tablet provided herein comprises a compound of Formula Ia, or a pharmaceutically acceptable salt thereof, and one or more pharmaceutically acceptable excipients. In some embodiments, the tablet provided herein comprises a compound of Formula Ib, or a pharmaceutically acceptable salt thereof, and one or more pharmaceutically acceptable excipients.

In some embodiments, the active ingredient is a compound of Formula Ia, or a pharmaceutically acceptable salt thereof. In some embodiments, the active ingredient is a compound of Formula Ib, or a pharmaceutically acceptable salt thereof.

In some embodiments, the active ingredient is a compound of Formula Ia (i.e., a free acid form of the compound of Formula Ia). In some embodiments, the active ingredient is a compound of Formula Ib (i.e., a free acid form of the compound of Formula Ia).

In some embodiments, the active ingredient is a crystalline form of a compound of Formula Ib (i.e., a crystalline form of Compound 1). In some embodiments, the active ingredient is crystalline Form I of the compound of Formula Ib (i.e., Compound 1, crystalline Form I).

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. In some embodiments, the crystalline form provided herein may be substantially anhydrous.

Crystalline forms can be identified by solid state characterization methods such as by X-ray powder diffraction (XRPD). Other characterization methods such as differential scanning calorimetry (DSC) further help identify the form as well as help determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 2-theta values. Thus, peak assignments, such as those reported herein, can vary by plus or minus about 0.2° (2-theta), and the term “substantially” and “about” as used in the context of XRPD herein is meant to encompass the above-mentioned variations.

In the same way, temperature readings in connection with DSC can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

In some embodiments, the compound of Formula Ib, crystalline Form I has at least one XRPD peak, in terms of 2-theta±0.2°, selected from 6.2°, 6.6°, 8.7°, 10.5°, 12.4°, 12.6°, 13.8°, 23.1°, and 25.7°.

In some embodiments, the compound of Formula Ib, crystalline Form I has at least two XRPD peaks, in terms of 2-theta±0.2°, selected from 6.2°, 6.6°, 8.7°, 10.5°, 12.4°, 12.6°, 13.8°, 23.1°, and 25.7°.

In some embodiments, the compound of Formula Ib, crystalline Form I has at least three XRPD peaks, in terms of 2-theta±0.2°, selected from 6.2°, 6.6°, 8.7°, 10.5°, 12.4°, 12.6°, 13.8°, 23.1°, and 25.7°.

In some embodiments, the compound of Formula Ib, crystalline Form I has at least four XRPD peaks, in terms of 2-theta±0.2°, selected from 6.2°, 6.6°, 8.7°, 10.5°, 12.4°, 12.6°, 13.8°, 23.1°, and 25.7°.

In some embodiments, the compound of Formula Ib, crystalline Form I has at least five XRPD peaks, in terms of 2-theta±0.2°, selected from 6.2°, 6.6°, 8.7°, 10.5°, 12.4°, 12.6°, 13.8°, 23.1°, and 25.7°.

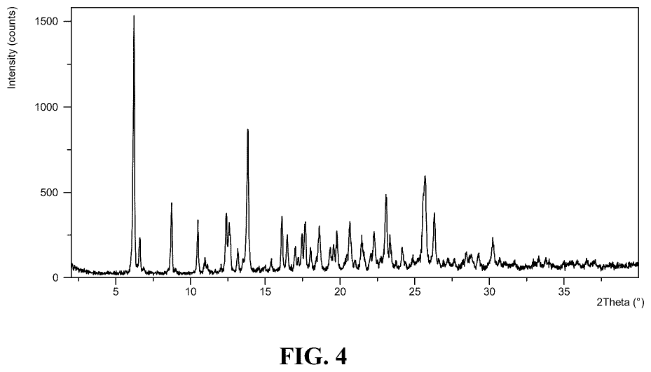

In some embodiments, the compound of Formula Ib, crystalline Form I is characterized by an XRPD pattern substantially as shown in FIG. 4.

In some embodiments, the compound of Formula Ib, crystalline Form I is characterized by a DSC thermogram having a melting onset at about 202° C.

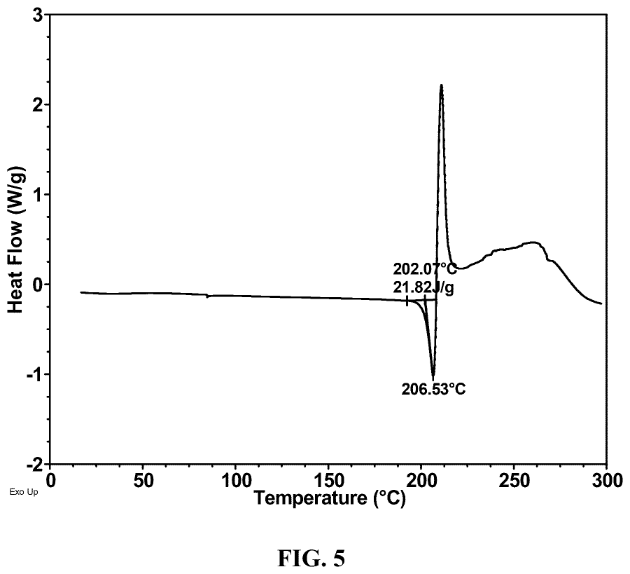

In some embodiments, the compound of Formula Ib, crystalline Form I is characterized by a DSC thermogram substantially as shown in FIG. 5.

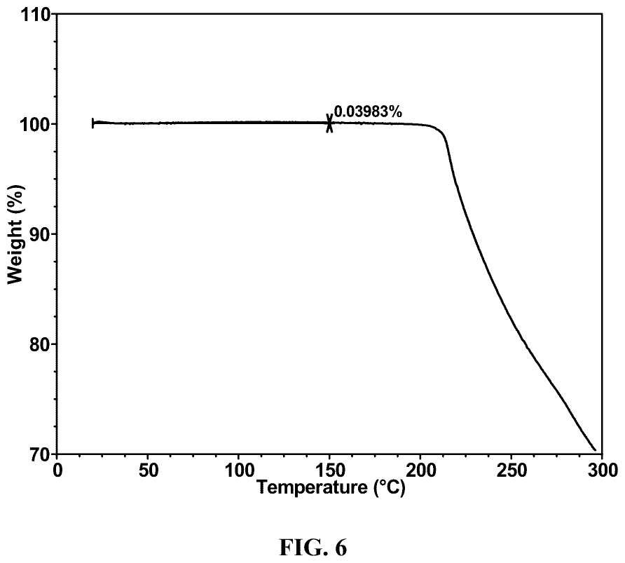

In some embodiments, the compound of Formula Ib, crystalline Form I is characterized by a TGA thermogram substantially as shown in FIG. 6.

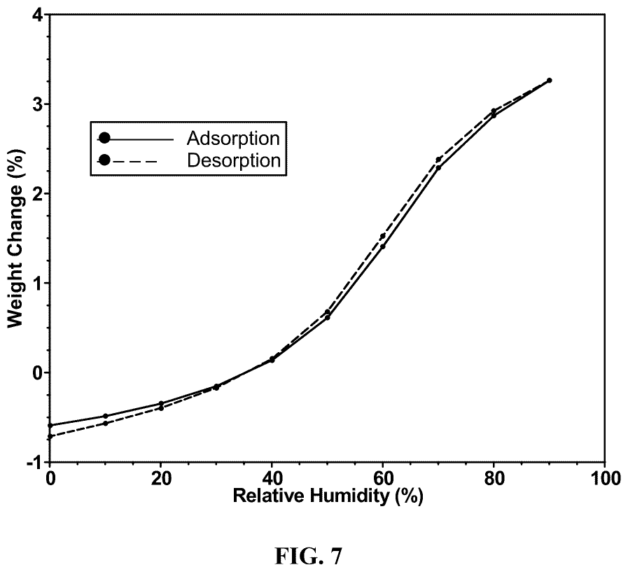

In some embodiments, the compound of Formula Ib, crystalline Form I is characterized by a DVS analysis substantially as shown in FIG. 7.

In some embodiments, the tablet disclosed herein comprise a compound of Formula Ia or Ib, or a pharmaceutically acceptable salt thereof, and one or more pharmaceutically acceptable excipients selected from the group consisting of a diluent, a disintegrant, and a lubricant.

In some embodiments, the tablet provided herein comprises about 30 w/w % to about 85 w/w % of the compound of Formula Ia or Ib, or a pharmaceutically acceptable salt thereof, for example, about 30 w/w %, about 35 w/w %, about 40 w/w %, about 45 w/w %, about 50 w/w %, about 55 w/w %, about 60 w/w %, about 65 w/w %, about 70 w/w %, about 75 w/w %, about 80 w/w %, or about 85 w/w % of the compound of Formula Ia or Ib, or a pharmaceutically acceptable salt thereof. In some embodiments, the tablet provided herein comprises about 30 w/w % to about 85 w/w % of the compound of Formula Ia or Ib (i.e., a free acid form of the compound of Formula Ia or Ib). In some embodiments, the tablet provided herein comprises about 30 w/w % to about 85 w/w % of the compound of Formula Ia. In some embodiments, the tablet provided herein comprises about 30 w/w % to about 85 w/w % of the compound of Formula Ib. In some embodiments, the tablet provided herein comprises about 30 w/w % to about 85 w/w % of a crystalline form of the compound of Formula Ib. In some embodiments, the tablet provided herein comprises about 30 w/w % to about 85 w/w % of the compound of Formula Ib, crystalline Form I.

In some embodiments, the tablet provided herein comprises about 70 w/w % to about 80 w/w % of the compound of Formula Ia or Ib, or a pharmaceutically acceptable salt thereof, for example, about 71 w/w %, about 72 w/w %, about 73 w/w %, about 74 w/w %, about 75 w/w %, about 76 w/w %, about 77 w/w %, about 78 w/w %, about 79 w/w %, or about 80 w/w % of the compound of Formula Ia or Ib, or a pharmaceutically acceptable salt thereof. In some embodiments, the tablet provided herein comprises about 70 w/w % to about 80 w/w % of the compound of Formula Ia. In some embodiments, the tablet provided herein comprises about 70 w/w % to about 80 w/w % of the compound of Formula Ib. In some embodiments, the tablet provided herein comprises about 30 w/w % to about 85 w/w % of a crystalline form of the compound of Formula Ib. In some embodiments, the tablet provided herein comprises about 70 w/w % to about 80 w/w % of the compound of Formula Ib, crystalline Form I.

In some embodiments, the tablet provided herein comprises about 75 w/w % of the compound of Formula Ia or Ib, or a pharmaceutically acceptable salt thereof. In some embodiments, the tablet provided herein comprises about 75 w/w % of the compound of Formula Ia or Ib (i.e., a free acid form of the compound of Formula Ia or Ib). In some embodiments, the tablet provided herein comprises about 75 w/w % of the compound of Formula Ia. In some embodiments, the tablet provided herein comprises about 75 w/w % of the compound of Formula Ib. In some embodiments, the tablet provided herein comprises about 75 w/w % of a crystalline form of the compound of Formula Ib. In some embodiments, the tablet provided herein comprises about 75 w/w % of the compound of Formula Ib, crystalline Form I.

In some embodiments, the tablet provided herein comprises about 30 w/w % to about 50 w/w % of the compound of Formula Ia or Ib, or a pharmaceutically acceptable salt thereof, for example, about 30 w/w %, about 35 w/w %, about 40 w/w %, about 45 w/w %, or about 50 w/w %. In some embodiments, the tablet provided herein comprises about 30 w/w % to about 50 w/w % of a free acid form of the compound of Formula Ia or Ib. In some embodiments, the tablet provided herein comprises about 30 w/w % to about 50 w/w % of a free acid form of the compound of Formula Ia. In some embodiments, the tablet provided herein comprises about 30 w/w % to about 50 w/w % of a free acid form of the compound of Formula Ib. In some embodiments, the tablet provided herein comprises about 30 w/w % to about 50 w/w % of a crystalline form of the compound of Formula Ib. In some embodiments, the tablet provided herein comprises about 30 w/w % to about 50 w/w % of the compound of Formula Ib, crystalline Form I.

In some embodiments, the tablet comprises about 35 w/w % to about 45 w/w % of the compound of Formula Ia or Ib, or a pharmaceutically acceptable salt thereof. In some embodiments, the tablet comprises about 35 w/w % to about 45 w/w % of the compound of Formula Ia, or a pharmaceutically acceptable salt thereof. In some embodiments, the tablet comprises about 35 w/w % to about 45 w/w % of the compound of Formula Ia. In some embodiments, the tablet comprises about 35 w/w % to about 45 w/w % of the compound of Formula Ib, or a pharmaceutically acceptable salt thereof. In some embodiments, the tablet comprises about 35 w/w % to about 45 w/w % of a free acid form of the compound of Formula Ib. In some embodiments, the tablet comprises about 35 w/w % to about 45 w/w % of a crystalline form of the compound of Formula Ib. In some embodiments, the tablet comprises about 35 w/w % to about 45 w/w % of the compound of Formula Ib, crystalline Form I.

In some embodiments, the tablet comprises about 40 w/w % of the compound of Formula Ia or Ib, or a pharmaceutically acceptable salt thereof. In some embodiments, the tablet comprises about 40 w/w % of the compound of Formula Ia, or a pharmaceutically acceptable salt thereof. In some embodiments, the tablet comprises about 40 w/w % of a free acid form of the compound of Formula Ia. In some embodiments, the tablet comprises about 40 w/w % of the compound of Formula Ib, or a pharmaceutically acceptable salt thereof. In some embodiments, the tablet comprises about 40 w/w % of a free acid form of the compound of Formula Ib. In some embodiments, the tablet comprises about 40 w/w % of a crystalline form of the compound of Formula Ib. In some embodiments, the tablet comprises about 40 w/w % of the compound of Formula Ib, crystalline Form I.

In some embodiments, the tablet comprises about 5 w/w % to about 35 w/w % of mannitol, for example, about 5 w/w % about 10 w/w % about 15 w/w % about 20 w/w % about 25 w/w % about 30 w/w % or about 30 w/w % of mannitol.

In some embodiments, the tablet comprises about 5 w/w % to about 10 w/w % of mannitol, for example, about 5%, about 6%, about 7%, about 8%, about 9%, or about 10% of mannitol. In some embodiments, the tablet comprises about 6 w/w % to about 9 w/w % of mannitol. In some embodiments, the tablet comprises about 8 w/w % of mannitol. In some embodiments, the tablet comprises about 7.7 w/w % of mannitol. In some embodiments, the tablet comprises about 7.8 w/w % of mannitol. In some embodiments, the tablet comprises about 7.75 w/w % of mannitol.

In some embodiments, the tablet comprises about 25 w/w % to about 35 w/w % of mannitol, for example, about 25 w/w %, about 26 w/w %, about 27 w/w %, about 28 w/w %, about 29 w/w %, about 30 w/w %, about 31 w/w %, about 32 w/w %, about 33 w/w %, about 34 w/w %, or about 35 w/w % mannitol.

In some embodiments, the tablet comprises about 25 w/w % to about 30 w/w % of mannitol. In some embodiments, the tablet comprises about 27.0 w/w % to about 28 w/w % of mannitol. In some embodiments, the tablet comprises about 27 w/w % of mannitol. In some embodiments, the tablet comprises about 27.0 w/w % to about 27.75 w/w % of mannitol. In some embodiments, the tablet comprises about 27.5 w/w % of mannitol. In some embodiments, the tablet comprises about 27.1 w/w % to about 27.6 w/w % of mannitol. In some embodiments, the tablet comprises about 27.2 w/w % to about 27.3 w/w % of mannitol. In some embodiments, the tablet comprises about 27.3 w/w % of mannitol. In some embodiments, the tablet comprises about 27.2 w/w % of mannitol. In some embodiments, the tablet comprises about 27.25 w/w % of mannitol.

In some embodiments, the tablet comprises about 5 w/w % to about 35 w/w % of microcrystalline cellulose, for example, about 5 w/w % about 10 w/w % about 15 w/w % about 20 w/w % about 25 w/w % about 30 w/w % or about 30 w/w % of microcrystalline cellulose.

In some embodiments, the tablet comprises about 5 w/w % to about 10 w/w % of microcrystalline cellulose, for example, about 5%, about 6%, about 7%, about 8%, about 9%, or about 10% of microcrystalline cellulose. In some embodiments, the tablet comprises about 6 w/w % to about 9 w/w % of microcrystalline cellulose. In some embodiments, the tablet comprises about 8 w/w % of microcrystalline cellulose. In some embodiments, the tablet comprises about 7.7 w/w % of microcrystalline cellulose. In some embodiments, the tablet comprises about 7.8 w/w % of microcrystalline cellulose. In some embodiments, the tablet comprises about 7.75 w/w % of microcrystalline cellulose.

In some embodiments, the tablet comprises about 25 w/w % to about 35 w/w % of microcrystalline cellulose, for example, about 25 w/w %, about 26 w/w %, about 27 w/w %, about 28 w/w %, about 29 w/w %, about 30 w/w %, about 31 w/w %, about 32 w/w %, about 33 w/w %, about 34 w/w %, or about 35 w/w % microcrystalline cellulose. In some embodiments, the tablet comprises about 25 w/w % to about 30 w/w % of microcrystalline cellulose. In some embodiments, the tablet comprises about 27.0 w/w % to about 28 w/w % of microcrystalline cellulose. In some embodiments, the tablet comprises about 27.0 w/w % to about 27.75 w/w % of microcrystalline cellulose. In some embodiments, the tablet comprises about 27.5 w/w % of microcrystalline cellulose. In some embodiments, the tablet comprises about 27.1 w/w % to about 27.6 w/w % of microcrystalline cellulose. In some embodiments, the tablet comprises about 27.2 w/w % to about 27.3 w/w % of microcrystalline cellulose. In some embodiments, the tablet comprises about 27.3 w/w % of microcrystalline cellulose. In some embodiments, the tablet comprises about 27.2 w/w % of microcrystalline cellulose. In some embodiments, the tablet comprises about 27.25 w/w % of microcrystalline cellulose.

In some embodiments, the tablet comprises about 1 w/w % to about 10 w/w % of crospovidone, for example, about 1%, about 2%, about 3%, about 4%, about 5%, about 6%, about 7%, about 8%, about 9%, or about 10% crospovidone. In some embodiments, the tablet comprises about 2 w/w % to about 6 w/w % of crospovidone. In some embodiments, the tablet comprises about 4 w/w % of crospovidone.

In some embodiments, the tablet comprises about 5 w/w % to about 10 w/w % of crospovidone. In some embodiments, the tablet comprises about 6 w/w % to about 10 w/w % of crospovidone. In some embodiments, the tablet comprises about 6 w/w % to about 9 w/w % of crospovidone. In some embodiments, the tablet comprises about 7 w/w % to about 9 w/w % of crospovidone. In some embodiments, the tablet comprises about 8 w/w % of crospovidone.

In some embodiments, the tablet comprises about 1 w/w % to about 5 w/w % of magnesium stearate, for example, about 1%, about 2%, about 3%, about 4%, or about 5% magnesium stearate. In some embodiments, the tablet comprises about 1 w/w % to about 3 w/w % of magnesium stearate. In some embodiments, the tablet comprises about 1.5 w/w % of magnesium stearate.

In some embodiments, the tablet provided herein comprises:
about 30 w/w % to about 50 w/w % of the compound of Formula Ia or Ib, or a pharmaceutically acceptable salt thereof;
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises:
about 35 w/w % to about 45 w/w % of the compound of Formula Ia or Ib, or a pharmaceutically acceptable salt thereof;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ia or Ib, or a pharmaceutically acceptable salt thereof;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises:
about 35 w/w % to about 45 w/w % of the compound of Formula Ia or Ib, or a pharmaceutically acceptable salt thereof;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ia or Ib, or a pharmaceutically acceptable salt thereof;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ia or Ib, or a pharmaceutically acceptable salt thereof,
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ia or Ib, or a pharmaceutically acceptable salt thereof,
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ia or Ib, or a pharmaceutically acceptable salt thereof;
about 27.25 w/w % of mannitol;
about 27.25 w/w % of microcrystalline cellulose;
about 4 w/w % of crospovidone; and
about 1.5 w/w % of magnesium stearate.

In some embodiments, the tablet provided herein comprises:
about 30 w/w % to about 50 w/w % of the compound of Formula Ia, or a pharmaceutically acceptable salt thereof;
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises:
about 35 w/w % to about 45 w/w % of the compound of Formula Ia, or a pharmaceutically acceptable salt thereof;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ia, or a pharmaceutically acceptable salt thereof;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises:
about 35 w/w % to about 45 w/w % of the compound of Formula Ia, or a pharmaceutically acceptable salt thereof;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ia, or a pharmaceutically acceptable salt thereof;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ia, or a pharmaceutically acceptable salt thereof;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ia, or a pharmaceutically acceptable salt thereof,
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ia, or a pharmaceutically acceptable salt thereof;
about 27.25 w/w % of mannitol;
about 27.25 w/w % of microcrystalline cellulose;
about 4 w/w % of crospovidone; and
about 1.5 w/w % of magnesium stearate.

In some embodiments, the tablet provided herein comprises:
about 30 w/w % to about 50 w/w % of the compound of Formula Ia (i.e., the free acid form of the compound of Formula Ia);
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises:
about 35 w/w % to about 45 w/w % of the compound of Formula Ia;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ia;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises:
about 35 w/w % to about 45 w/w % of the compound of Formula Ia;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ia;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ia;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ia;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ia;
about 27.25 w/w % of mannitol;
about 27.25 w/w % of microcrystalline cellulose;
about 4 w/w % of crospovidone; and
about 1.5 w/w % of magnesium stearate.

In some embodiments, the tablet provided herein comprises:
about 30 w/w % to about 50 w/w % of the compound of Formula Ib, or a pharmaceutically acceptable salt thereof;
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises:
about 35 w/w % to about 45 w/w % of the compound of Formula Ib, or a pharmaceutically acceptable salt thereof;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ib, or a pharmaceutically acceptable salt thereof;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises:
about 35 w/w % to about 45 w/w % of the compound of Formula Ib, or a pharmaceutically acceptable salt thereof;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ib, or a pharmaceutically acceptable salt thereof,
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ib, or a pharmaceutically acceptable salt thereof;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ib, or a pharmaceutically acceptable salt thereof;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ib, or a pharmaceutically acceptable salt thereof,
about 27.25 w/w % of mannitol;
about 27.25 w/w % of microcrystalline cellulose;
about 4 w/w % of crospovidone; and
about 1.5 w/w % of magnesium stearate.

In some embodiments, the tablet provided herein comprises:
about 30 w/w % to about 50 w/w % of the compound of Formula Ib (i.e., the free acid form of the compound of Formula Ib);
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises:
about 35 w/w % to about 45 w/w % of the compound of Formula Ib;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ib;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises:
about 35 w/w % to about 45 w/w % of the compound of Formula Ib;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ib;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ib;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ib;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ib;
about 27.25 w/w % of mannitol;
about 27.25 w/w % of microcrystalline cellulose;
about 4 w/w % of crospovidone; and
about 1.5 w/w % of magnesium stearate.

In some embodiments, the tablet provided herein comprises:
about 30 w/w % to about 50 w/w % of a crystalline form of the compound of Formula Ib (i.e., a crystalline form of the free acid form of the compound of Formula Ib);
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises:
about 35 w/w % to about 45 w/w % of a crystalline form of the compound of Formula Ib;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of a crystalline form of the compound of Formula Ib;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises:
about 35 w/w % to about 45 w/w % of a crystalline form of the compound of Formula Ib;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of a crystalline form of the compound of Formula Ib;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of a crystalline form of the compound of Formula Ib;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of a crystalline form of the compound of Formula Ib;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of a crystalline form of the compound of Formula Ib;
about 27.25 w/w % of mannitol;
about 27.25 w/w % of microcrystalline cellulose;
about 4 w/w % of crospovidone; and
about 1.5 w/w % of magnesium stearate.

In some embodiments, the tablet provided herein comprises:
about 30 w/w % to about 50 w/w % of the compound of Formula Ib, crystalline Form I;
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

In some embodiments, the tablet provided herein comprises:
about 35 w/w % to about 45 w/w % of the compound of Formula Ib, crystalline Form I;
about 25 w/w % to about 30 w/w % of mannitol;
about 25 w/w % to about 30 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ib, crystalline Form I;
about 27 w/w % of mannitol;
about 27.5 w/w % of microcrystalline cellulose;
about 4 w/w % of crospovidone; and
about 1.5 w/w % of magnesium stearate.

In some embodiments, the tablet provided herein comprises:
about 35 w/w % to about 45 w/w % of the compound of Formula Ib, crystalline Form I;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ib, crystalline Form I;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ib, crystalline Form I;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 35 w/w % to about 45 w/w % of the compound of Formula Ib, crystalline Form I;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the tablet provided herein comprises:
about 40 w/w % of the compound of Formula Ib, crystalline Form I;
about 27.25 w/w % of mannitol;
about 27.25 w/w % of microcrystalline cellulose;
about 4 w/w % of crospovidone; and
about 1.5 w/w % of magnesium stearate.

In some embodiments, the tablet provided herein comprises:
about 70 w/w % to about 80 w/w % of the compound of Formula Ia or Ib, or a pharmaceutically acceptable salt thereof;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the tablet provided herein comprises:
about 70 w/w % to about 80 w/w % of the compound of Formula Ia or Ib, or a pharmaceutically acceptable salt thereof;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate

In some embodiments, the tablet provided herein comprises:
about 75 w/w % of the compound of Formula Ia or Ib, or a pharmaceutically acceptable salt thereof;
about 7.8 w/w % of mannitol;
about 7.8 w/w % of microcrystalline cellulose;
about 8 w/w % of crospovidone; and
about 1.5 w/w % of magnesium stearate.

In some embodiments, the tablet provided herein comprises:
about 75 w/w % of the compound of Formula Ia or Ib, or a pharmaceutically acceptable salt thereof;
about 7.75 w/w % of mannitol;
about 7.75 w/w % of microcrystalline cellulose;
about 8 w/w % of crospovidone; and
about 1.5 w/w % of magnesium stearate.

In some embodiments, the tablet provided herein comprises:
about 70 w/w % to about 80 w/w % of the compound of Formula Ia, or a pharmaceutically acceptable salt thereof;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the tablet provided herein comprises:
about 70 w/w % to about 80 w/w % of the compound of Formula Ia, or a pharmaceutically acceptable salt thereof;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate

In some embodiments, the tablet provided herein comprises:
about 75 w/w % of the compound of Formula Ia, or a pharmaceutically acceptable salt thereof;
about 7.8 w/w % of mannitol;
about 7.8 w/w % of microcrystalline cellulose;
about 8 w/w % of crospovidone; and
about 1.5 w/w % of magnesium stearate.

In some embodiments, the tablet provided herein comprises:
about 75 w/w % of the compound of Formula Ia, or a pharmaceutically acceptable salt thereof;
about 7.75 w/w % of mannitol;
about 7.75 w/w % of microcrystalline cellulose;
about 8 w/w % of crospovidone; and
about 1.5 w/w % of magnesium stearate.

In some embodiments, the tablet provided herein comprises:
about 70 w/w % to about 80 w/w % of the compound of Formula Ia;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the tablet provided herein comprises:
about 70 w/w % to about 80 w/w % of the compound of Formula Ia;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate

In some embodiments, the tablet provided herein comprises:
about 75 w/w % of the compound of Formula Ia;
about 7.8 w/w % of mannitol;
about 7.8 w/w % of microcrystalline cellulose;
about 8 w/w % of crospovidone; and
about 1.5 w/w % of magnesium stearate.

In some embodiments, the tablet provided herein comprises:
about 75 w/w % of the compound of Formula Ia;
about 7.75 w/w % of mannitol;
about 7.75 w/w % of microcrystalline cellulose;
about 8 w/w % of crospovidone; and
about 1.5 w/w % of magnesium stearate.

In some embodiments, the tablet provided herein comprises:
about 70 w/w % to about 80 w/w % of the compound of Formula Ib, or a pharmaceutically acceptable salt thereof;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the tablet provided herein comprises:
about 70 w/w % to about 80 w/w % of the compound of Formula Ib, or a pharmaceutically acceptable salt thereof;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate

In some embodiments, the tablet provided herein comprises:
about 75 w/w % of the compound of Formula Ib, or a pharmaceutically acceptable salt thereof;
about 7.8 w/w % of mannitol;
about 7.8 w/w % of microcrystalline cellulose;
about 8 w/w % of crospovidone; and
about 1.5 w/w % of magnesium stearate.

In some embodiments, the tablet provided herein comprises:
about 75 w/w % of the compound of Formula Ib, or a pharmaceutically acceptable salt thereof;
about 7.75 w/w % of mannitol;
about 7.75 w/w % of microcrystalline cellulose;
about 8 w/w % of crospovidone; and
about 1.5 w/w % of magnesium stearate.

In some embodiments, the tablet provided herein comprises:
about 70 w/w % to about 80 w/w % of the compound of Formula Ib;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the tablet provided herein comprises:
about 70 w/w % to about 80 w/w % of the compound of Formula Ib;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate

In some embodiments, the tablet provided herein comprises:
about 75 w/w % of the compound of Formula Ib;
about 7.8 w/w % of mannitol;
about 7.8 w/w % of microcrystalline cellulose;
about 8 w/w % of crospovidone; and
about 1.5 w/w % of magnesium stearate.

In some embodiments, the tablet provided herein comprises:
about 75 w/w % of the compound of Formula Ib;
about 7.75 w/w % of mannitol;
about 7.75 w/w % of microcrystalline cellulose;
about 8 w/w % of crospovidone; and
about 1.5 w/w % of magnesium stearate.

In some embodiments, the tablet provided herein comprises:
about 70 w/w % to about 80 w/w % of a crystalline form of the compound of Formula Ib;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the tablet provided herein comprises:
about 70 w/w % to about 80 w/w % of a crystalline form of the compound of Formula Ib;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate

In some embodiments, the tablet provided herein comprises:
about 75 w/w % of a crystalline form of the compound of Formula Ib;
about 7.8 w/w % of mannitol;
about 7.8 w/w % of microcrystalline cellulose;
about 8 w/w % of crospovidone; and
about 1.5 w/w % of magnesium stearate.

In some embodiments, the tablet provided herein comprises:
about 75 w/w % of a crystalline form of the compound of Formula Ib;
about 7.75 w/w % of mannitol;
about 7.75 w/w % of microcrystalline cellulose;
about 8 w/w % of crospovidone; and
about 1.5 w/w % of magnesium stearate.

In some embodiments, the tablet provided herein comprises:
about 70 w/w % to about 80 w/w % of a compound of Formula Ib, crystalline Form I;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the tablet provided herein comprises:
about 70 w/w % to about 80 w/w % of a compound of Formula Ib, crystalline Form I;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate

In some embodiments, the tablet provided herein comprises:
about 75 w/w % of a compound of Formula Ib, crystalline Form I;
about 7.8 w/w % of mannitol;
about 7.8 w/w % of microcrystalline cellulose;
about 8 w/w % of crospovidone; and
about 1.5 w/w % of magnesium stearate.

In some embodiments, the tablet provided herein comprises:
about 75 w/w % of a compound of Formula Ib, crystalline Form I;
about 7.75 w/w % of mannitol;
about 7.75 w/w % of microcrystalline cellulose;
about 8 w/w % of crospovidone; and
about 1.5 w/w % of magnesium stearate.

In some embodiments, the tablet comprises about 1 mg to about 2000 mg of the compound of Formula Ia or Ib, or a pharmaceutically acceptable salt thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1600 mg, about 1700 mg, about 1800 mg, about 1900 mg, or about 2000 mg, of the compound of Formula Ia or Ib, or a pharmaceutically acceptable salt thereof.

In some embodiments, the tablet comprises about 1000 mg to about 1500 mg of the compound of Formula Ia or Ib, or a pharmaceutically acceptable salt thereof. In some embodiments, the tablet comprises about 1100 mg to about 1300 mg of the compound of Formula Ia or Ib, or a pharmaceutically acceptable salt thereof. In some embodiments, the tablet comprises about 1175 mg to about 1225 mg of the compound of Formula Ia or Ib, or a pharmaceutically acceptable salt thereof. In some embodiments, the tablet comprises about 1200 mg of the compound of Formula Ia or Ib, or a pharmaceutically acceptable salt thereof.

In some embodiments, the tablet comprises about 1000 mg to about 1500 mg of the compound of Formula Ia, or a pharmaceutically acceptable salt thereof. In some embodiments, the tablet comprises about 1100 mg to about 1300 mg of the compound of Formula Ia, or a pharmaceutically acceptable salt thereof. In some embodiments, the tablet comprises about 1175 mg to about 1225 mg of the compound of Formula Ia, or a pharmaceutically acceptable salt thereof. In some embodiments, the tablet comprises about 1200 mg of the compound of Formula Ia, or a pharmaceutically acceptable salt thereof.

In some embodiments, the tablet comprises about 1000 mg to about 1500 mg of the compound of Formula Ia. In some embodiments, the tablet comprises about 1100 mg to about 1300 mg of the compound of Formula Ia. In some embodiments, the tablet comprises about 1175 mg to about 1225 mg of the compound of Formula Ia. In some embodiments, the tablet comprises about 1200 mg of the compound of Formula Ia.

In some embodiments, the tablet comprises about 1000 mg to about 1500 mg of the compound of Formula Ib, or a pharmaceutically acceptable salt thereof. In some embodiments, the tablet comprises about 1100 mg to about 1300 mg of the compound of Formula Ib, or a pharmaceutically acceptable salt thereof. In some embodiments, the tablet comprises about 1175 mg to about 1225 mg of the compound of Formula Ib, or a pharmaceutically acceptable salt thereof. In some embodiments, the tablet comprises about 1200 mg of the compound of Formula Ib, or a pharmaceutically acceptable salt thereof.

In some embodiments, the tablet comprises about 1000 mg to about 1500 mg of the compound of Formula Ib. In some embodiments, the tablet comprises about 1100 mg to about 1300 mg of the compound of Formula Ib. In some embodiments, the tablet comprises about 1175 mg to about 1225 mg of the compound of Formula Ib. In some embodiments, the tablet comprises about 1200 mg of the compound of Formula Ib.

In some embodiments, the tablet comprises about 1000 mg to about 1500 mg of a crystalline form of the compound of Formula Ib. In some embodiments, the tablet comprises about 1100 mg to about 1300 mg of a crystalline form of the compound of Formula Ib. In some embodiments, the tablet comprises about 1175 mg to about 1225 mg of a crystalline form of the compound of Formula Ib. In some embodiments, the tablet comprises about 1200 mg of a crystalline form of the compound of Formula Ib.

In some embodiments, the tablet comprises about 1000 mg to about 1500 mg of a compound of Formula Ib, crystalline Form I. In some embodiments, the tablet comprises about 1100 mg to about 1300 mg of a compound of Formula Ib, crystalline Form I. In some embodiments, the tablet comprises about 1175 mg to about 1225 mg of a compound of Formula Ib, crystalline Form I. In some embodiments, the tablet comprises about 1200 mg of a compound of Formula Ib, crystalline Form I.

In some embodiments, the tablet comprises about 1 mg to about 500 mg of the compound of Formula Ia or Ib, or a pharmaceutically acceptable salt thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound of Formula Ia or Ib, or a pharmaceutically acceptable salt thereof.

In some embodiments, the tablet comprises about 5 mg to about 500 mg of the compound of Formula Ia or Ib, or a pharmaceutically acceptable salt thereof. In some embodiments, the tablet comprises about 50 mg to about 450 mg of the compound of Formula Ia or Ib, or a pharmaceutically acceptable salt thereof. In some embodiments, the tablet comprises about 75 mg to about 425 mg of the compound of Formula Ia or Ib, or a pharmaceutically acceptable salt thereof.

In some embodiments, the tablet comprises about 75 mg to about 125 mg of the compound of Formula Ia or Ib, or a pharmaceutically acceptable salt thereof. In some embodiments, the tablet comprises about 100 mg of the compound of Formula Ia or Ib, or a pharmaceutically acceptable salt thereof.

In some embodiments, the tablet comprises about 175 mg to about 225 mg of the compound of Formula Ia or Ib, or a pharmaceutically acceptable salt thereof. In some embodiments, the tablet comprises about 200 mg of the compound of Formula Ia or Ib, or a pharmaceutically acceptable salt thereof.

In some embodiments, the tablet comprises about 275 mg to about 325 mg of the compound of Formula Ia or Ib, or a pharmaceutically acceptable salt thereof. In some embodiments, the tablet comprises about 300 mg of the compound of Formula Ia or Ib, or a pharmaceutically acceptable salt thereof.

In some embodiments, the tablet comprises about 375 mg to about 425 mg of the compound of Formula Ia or Ib, or a pharmaceutically acceptable salt thereof. In some embodiments, the tablet comprises about 400 mg of the compound of Formula Ia or Ib, or a pharmaceutically acceptable salt thereof.

In some embodiments, the tablet comprises about 1 mg to about 500 mg of the compound of Formula Ia, or a pharmaceutically acceptable salt thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound of Formula Ia, or a pharmaceutically acceptable salt thereof.

In some embodiments, the tablet comprises about 5 mg to about 500 mg of the compound of Formula Ia, or a pharmaceutically acceptable salt thereof. In some embodiments, the tablet comprises about 50 mg to about 450 mg of the compound of Formula Ia, or a pharmaceutically acceptable salt thereof. In some embodiments, the tablet comprises about 75 mg to about 425 mg of the compound of Formula Ia, or a pharmaceutically acceptable salt thereof.

In some embodiments, the tablet comprises about 75 mg to about 125 mg of the compound of Formula Ia, or a pharmaceutically acceptable salt thereof. In some embodiments, the tablet comprises about 100 mg of the compound of Formula Ia, or a pharmaceutically acceptable salt thereof.

In some embodiments, the tablet comprises about 175 mg to about 225 mg of the compound of Formula Ia, or a pharmaceutically acceptable salt thereof. In some embodiments, the tablet comprises about 200 mg of the compound of Formula Ia, or a pharmaceutically acceptable salt thereof.

In some embodiments, the tablet comprises about 275 mg to about 325 mg of the compound of Formula Ia, or a pharmaceutically acceptable salt thereof. In some embodiments, the tablet comprises about 300 mg of the compound of Formula Ia, or a pharmaceutically acceptable salt thereof.

In some embodiments, the tablet comprises about 375 mg to about 425 mg of the compound of Formula Ia, or a pharmaceutically acceptable salt thereof. In some embodiments, the tablet comprises about 400 mg of the compound of Formula Ia, or a pharmaceutically acceptable salt thereof.

In some embodiments, the tablet comprises about 1 mg to about 500 mg of the compound of Formula Ib, or a pharmaceutically acceptable salt thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound of Formula Ib, or a pharmaceutically acceptable salt thereof.

In some embodiments, the tablet comprises about 5 mg to about 500 mg of the compound of Formula Ib, or a pharmaceutically acceptable salt thereof. In some embodiments, the tablet comprises about 50 mg to about 450 mg of the compound of Formula Ib, or a pharmaceutically acceptable salt thereof. In some embodiments, the tablet comprises about 75 mg to about 425 mg of the compound of Formula Ib, or a pharmaceutically acceptable salt thereof.

In some embodiments, the tablet comprises about 75 mg to about 125 mg of the compound of Formula Ib, or a pharmaceutically acceptable salt thereof. In some embodiments, the tablet comprises about 100 mg of the compound of Formula Ib, or a pharmaceutically acceptable salt thereof.

In some embodiments, the tablet comprises about 175 mg to about 225 mg of the compound of Formula Ib, or a pharmaceutically acceptable salt thereof. In some embodiments, the tablet comprises about 200 mg of the compound of Formula Ib, or a pharmaceutically acceptable salt thereof.

In some embodiments, the tablet comprises about 275 mg to about 325 mg of the compound of Formula Ib, or a pharmaceutically acceptable salt thereof. In some embodiments, the tablet comprises about 300 mg of the compound of Formula Ib, or a pharmaceutically acceptable salt thereof.

In some embodiments, the tablet comprises about 375 mg to about 425 mg of the compound of Formula Ib, or a pharmaceutically acceptable salt thereof. In some embodiments, the tablet comprises about 400 mg of the compound of Formula Ib, or a pharmaceutically acceptable salt thereof.

In some embodiments, the tablet comprises about 1 mg to about 500 mg of a crystalline form of the compound of Formula Ib, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of a crystalline form of the compound of Formula Ib.

In some embodiments, the tablet comprises about 5 mg to about 500 mg of a crystalline form of the compound of Formula Ib. In some embodiments, the tablet comprises about 50 mg to about 450 mg of a crystalline form of the compound of Formula Ib. In some embodiments, the tablet comprises about 75 mg to about 425 mg of a crystalline form of the compound of Formula Ib.

In some embodiments, the tablet comprises about 75 mg to about 125 mg of a crystalline form of the compound of Formula Ib. In some embodiments, the tablet comprises about 100 mg of a crystalline form of the compound of Formula Ib.

In some embodiments, the tablet comprises about 175 mg to about 225 mg of a crystalline form of the compound of Formula Ib. In some embodiments, the tablet comprises about 200 mg of a crystalline form of the compound of Formula Ib.

In some embodiments, the tablet comprises about 275 mg to about 325 mg of a crystalline form of the compound of Formula Ib. In some embodiments, the tablet comprises about 300 mg of a crystalline form of the compound of Formula Ib.

In some embodiments, the tablet comprises about 375 mg to about 425 mg of a crystalline form of the compound of Formula Ib. In some embodiments, the tablet comprises about 400 mg of a crystalline form of the compound of Formula Ib.

In some embodiments, the tablet comprises about 1 mg to about 500 mg of the compound of Formula Ib, crystalline Form I, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg of the compound of Formula Ib, crystalline Form I.

In some embodiments, the tablet comprises about 5 mg to about 500 mg of the compound of Formula Ib, crystalline Form I. In some embodiments, the tablet comprises about 50 mg to about 450 mg of the compound of Formula Ib, crystalline Form I. In some embodiments, the tablet comprises about 75 mg to about 425 mg of the compound of Formula Ib, crystalline Form I.

In some embodiments, the tablet comprises about 75 mg to about 125 mg of the compound of Formula Ib, crystalline Form I. In some embodiments, the tablet comprises about 100 mg of the compound of Formula Ib, crystalline Form I.

In some embodiments, the tablet comprises about 175 mg to about 225 mg of the compound of Formula Ib, crystalline Form I. In some embodiments, the tablet comprises about 200 mg of the compound of Formula Ib, crystalline Form I.

In some embodiments, the tablet comprises about 275 mg to about 325 mg of the compound of Formula Ib, crystalline Form I. In some embodiments, the tablet comprises about 300 mg of the compound of Formula Ib, crystalline Form I.

In some embodiments, the tablet comprises about 375 mg to about 425 mg of the compound of Formula Ib, crystalline Form I. In some embodiments, the tablet comprises about 400 mg of the compound of Formula Ib, crystalline Form I.

In some embodiments, the tablet comprises about 50 mg to about 300 mg of mannitol, for example, about 50 mg, about 100 mg, about 150 mg, about 200 mg, about 250 mg, about 275 mg, or about 300 mg of mannitol.

In some embodiments, the tablet comprises about 50 mg to about 100 mg of mannitol. In some embodiments, the tablet comprises about 55 mg to about 75 mg of mannitol. In some embodiments, the tablet comprises about 65 mg to about 75 mg of mannitol. In some embodiments, the tablet comprises about 67 mg to about 68 mg of mannitol. In some embodiments, the tablet comprises about 67.5 mg of mannitol.

In some embodiments, the tablet comprises about 100 mg to about 150 mg of mannitol. In some embodiments, the tablet comprises about 120 mg to about 140 mg of mannitol. In some embodiments, the tablet comprises about 130 mg to about 140 mg of mannitol. In some embodiments, the tablet comprises about 135 mg to about 140 mg of mannitol. In some embodiments, the tablet comprises about 135 mg to about 137 mg of mannitol. In some embodiments, the tablet comprises about 136 mg to about 137 mg of mannitol. In some embodiments, the tablet comprises about 134 mg to about 136 mg of mannitol. In some embodiments, the tablet comprises about 135 mg of mannitol. In some embodiments, the tablet comprises about 136 mg of mannitol. In some embodiments, the tablet comprises about 136.2 mg of mannitol. In some embodiments, the tablet comprises about 136.3 mg of mannitol. In some embodiments, the tablet comprises about 136.25 mg of mannitol.

In some embodiments, the tablet comprises about 120 mg to about 130 mg of mannitol. In some embodiments, the tablet comprises about 123 mg to about 125 mg of mannitol. In some embodiments, the tablet comprises about 124 mg of mannitol.

In some embodiments, the tablet comprises about 150 mg to about 250 mg of mannitol. In some embodiments, the tablet comprises about 190 mg to about 210 mg of mannitol. In some embodiments, the tablet comprises about 200 mg to about 205 mg of mannitol. In some embodiments, the tablet comprises about 202 mg to about 203 mg of mannitol. In some embodiments, the tablet comprises about 202.5 mg of mannitol.

In some embodiments, the tablet comprises about 250 mg to about 300 mg of mannitol. In some embodiments, the tablet comprises about 260 mg to about 280 mg of mannitol. In some embodiments, the tablet comprises about 265 mg to about 275 mg of mannitol. In some embodiments, the tablet comprises about 269 mg to about 271 mg of mannitol. In some embodiments, the tablet comprises about 270 mg of mannitol.

In some embodiments, the tablet comprises about 50 mg to about 300 mg of microcrystalline cellulose, for example, about 50 mg, about 100 mg, about 150 mg, about 200 mg, about 250 mg, about 275 mg, or about 300 mg of microcrystalline cellulose.

In some embodiments, the tablet comprises about 50 mg to about 100 mg of microcrystalline cellulose. In some embodiments, the tablet comprises about 55 mg to about 75 mg of microcrystalline cellulose. In some embodiments, the tablet comprises about 65 mg to about 75 mg of microcrystalline cellulose. In some embodiments, the tablet comprises about 68 mg to about 69 mg of microcrystalline cellulose. In some embodiments, the tablet comprises about 68.75 mg of microcrystalline cellulose.

In some embodiments, the tablet comprises about 100 mg to about 150 mg of microcrystalline cellulose. In some embodiments, the tablet comprises about 120 mg to about 140 mg of microcrystalline cellulose. In some embodiments, the tablet comprises about 135 mg to about 140 mg of microcrystalline cellulose. In some embodiments, the tablet comprises about 136 mg to about 138 mg of microcrystalline cellulose. In some embodiments, the tablet comprises about 136 mg to about 137 mg of microcrystalline cellulose. In some embodiments, the tablet comprises about 137.5 mg of microcrystalline cellulose. In some embodiments, the tablet comprises about 136.2 mg of microcrystalline cellulose. In some embodiments, the tablet comprises about 136.3 mg of microcrystalline cellulose. In some embodiments, the tablet comprises about 136.25 mg of microcrystalline cellulose.

In some embodiments, the tablet comprises about 120 mg to about 130 mg of microcrystalline cellulose. In some embodiments, the tablet comprises about 123 mg to about 125 mg of microcrystalline cellulose. In some embodiments, the tablet comprises about 124 mg of microcrystalline cellulose.

In some embodiments, the tablet comprises about 150 mg to about 250 mg of microcrystalline cellulose. In some embodiments, the tablet comprises about 195 mg to about 215 mg of microcrystalline cellulose. In some embodiments, the tablet comprises about 205 mg to about 210 mg of microcrystalline cellulose. In some embodiments, the tablet comprises about 206 mg to about 207 mg of microcrystalline cellulose. In some embodiments, the tablet comprises about 206.25 mg of microcrystalline cellulose.

In some embodiments, the tablet comprises about 250 mg to about 300 mg of microcrystalline cellulose. In some embodiments, the tablet comprises about 265 mg to about 285 mg of microcrystalline cellulose. In some embodiments, the tablet comprises about 270 mg to about 280 mg of microcrystalline cellulose. In some embodiments, the tablet comprises about 274 mg to about 276 mg of microcrystalline cellulose. In some embodiments, the tablet comprises about 275 mg of microcrystalline cellulose.

In some embodiments, the tablet comprises about 5 mg to about 150 mg of crospovidone, for example, about 5 mg, about 10 mg, about 15 mg, about 20 mg, about 25 mg, about 30 mg, about 35 mg, about 40 mg, about 45 mg, about 50 mg, about 75 mg, about 100 mg, about 125 mg, or about 150 mg of crospovidone.

In some embodiments, the tablet comprises about 100 mg to about 150 mg of crospovidone. In some embodiments, the tablet comprises about 120 mg to about 140 mg of crospovidone. In some embodiments, the tablet comprises about 125 mg to about 130 mg of crospovidone. In some embodiments, the tablet comprises about 127 mg to about 129 mg of crospovidone. In some embodiments, the tablet comprises about 128 mg of crospovidone.

In some embodiments, the tablet comprises about 5 mg to about 50 mg of crospovidone, for example, about 5 mg, about 10 mg, about 15 mg, about 20 mg, about 25 mg, about 30 mg, about 35 mg, about 40 mg, about 45 mg, or about 50 mg of crospovidone.

In some embodiments, the tablet comprises about 5 mg to about 15 mg of crospovidone. In some embodiments, the tablet comprises about 7 mg to about 13 mg of crospovidone. In some embodiments, the tablet comprises about 9 mg to about 11 mg of crospovidone. In some embodiments, the tablet comprises about 10 mg of crospovidone.

In some embodiments, the tablet comprises about 15 mg to about 25 mg of crospovidone. In some embodiments, the tablet comprises about 17 mg to about 23 mg of crospovidone. In some embodiments, the tablet comprises about 19 mg to about 21 mg of crospovidone. In some embodiments, the tablet comprises about 20 mg of crospovidone.

In some embodiments, the tablet comprises about 25 mg to about 35 mg of crospovidone. In some embodiments, the tablet comprises about 27 mg to about 33 mg of crospovidone. In some embodiments, the tablet comprises about 29 mg to about 31 mg of crospovidone. In some embodiments, the tablet comprises about 30 mg of crospovidone.

In some embodiments, the tablet comprises about 35 mg to about 45 mg of crospovidone. In some embodiments, the tablet comprises about 37 mg to about 43 mg of crospovidone. In some embodiments, the tablet comprises about 39 mg to about 41 mg of crospovidone. In some embodiments, the tablet comprises about 40 mg of crospovidone.

In some embodiments, the tablet comprises about 1 mg to about 30 mg of magnesium stearate, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, or about 30 mg of magnesium stearate.

In some embodiments, the tablet comprises about 20 mg to about 30 mg of magnesium stearate. In some embodiments, the tablet comprises about 22 mg to about 28 mg of magnesium stearate. In some embodiments, the tablet comprises about 23 mg to about 25 mg of magnesium stearate. In some embodiments, the tablet comprises about 24 mg of magnesium stearate.

In some embodiments, the tablet comprises about 1 mg to about 20 mg of magnesium stearate, for example, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg magnesium stearate.

In some embodiments, the tablet comprises about 1 mg to about 6 mg of magnesium stearate. In some embodiments, the tablet comprises about 2 mg to about 5 mg of magnesium stearate. In some embodiments, the tablet comprises about 3 mg to about 4 mg of magnesium stearate. In some embodiments, the tablet comprises about 3.75 mg of magnesium stearate.

In some embodiments, the tablet comprises about 5 mg to about 10 mg of magnesium stearate. In some embodiments, the tablet comprises about 6 mg to about 9 mg of magnesium stearate. In some embodiments, the tablet comprises about 6 mg to about 8 mg of magnesium stearate. In some embodiments, the tablet comprises about 7.5 mg of magnesium stearate.

In some embodiments, the tablet comprises about 9 mg to about 14 mg of magnesium stearate. In some embodiments, the tablet comprises about 10 mg to about 13 mg of magnesium stearate. In some embodiments, the tablet comprises about 11 mg to about 12 mg of magnesium stearate. In some embodiments, the tablet comprises about 11.25 mg of magnesium stearate.

In some embodiments, the tablet comprises about 10 mg to about 20 mg of magnesium stearate. In some embodiments, the tablet comprises about 13 mg to about 17 mg of magnesium stearate. In some embodiments, the tablet comprises about 14 mg to about 16 mg of magnesium stearate. In some embodiments, the tablet comprises about 15 mg of magnesium stearate.

In some embodiments, the tablet provided herein comprises:
about 5 mg to about 500 mg of the compound of Formula Ia or Ib, or a pharmaceutically acceptable salt thereof;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises:
about 75 mg to about 125 mg of the compound of Formula Ia or Ib, or a pharmaceutically acceptable salt thereof,
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the tablet provided herein comprises:
about 100 mg of the compound of Formula Ia or Ib, or a pharmaceutically acceptable salt thereof;
about 67.5 mg of mannitol;
about 68.75 mg of microcrystalline cellulose;
about 10 mg of crospovidone; and
about 3.75 mg of magnesium stearate.

In some embodiments, the tablet provided herein comprises:
about 75 mg to about 125 mg of the compound of Formula Ia, or a pharmaceutically acceptable salt thereof;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the tablet provided herein comprises:
about 100 mg of the compound of Formula Ia, or a pharmaceutically acceptable salt thereof;
about 67.5 mg of mannitol;
about 68.75 mg of microcrystalline cellulose;
about 10 mg of crospovidone; and
about 3.75 mg of magnesium stearate.

In some embodiments, the tablet provided herein comprises:
about 75 mg to about 125 mg of the compound of Formula Ib, or a pharmaceutically acceptable salt thereof;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the tablet provided herein comprises:
about 100 mg of the compound of Formula Ib, or a pharmaceutically acceptable salt thereof;
about 67.5 mg of mannitol;
about 68.75 mg of microcrystalline cellulose;
about 10 mg of crospovidone; and
about 3.75 mg of magnesium stearate.

In some embodiments, the tablet provided herein comprises:
about 75 mg to about 125 mg of a crystalline form of the compound of Formula Ib;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the tablet provided herein comprises:
about 100 mg of a crystalline form of the compound of Formula Ib;
about 67.5 mg of mannitol;
about 68.75 mg of microcrystalline cellulose;
about 10 mg of crospovidone; and
about 3.75 mg of magnesium stearate.

In some embodiments, the tablet provided herein comprises:
about 75 mg to about 125 mg of the compound of Formula Ib, crystalline Form I;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the tablet provided herein comprises:
about 100 mg of the compound of Formula Ib, crystalline Form I;
about 67.5 mg of mannitol;
about 68.75 mg of microcrystalline cellulose;
about 10 mg of crospovidone; and
about 3.75 mg of magnesium stearate.

In some embodiments, the tablet provided herein comprises:
about 175 mg to about 225 mg of the compound of Formula Ia or Ib, or a pharmaceutically acceptable salt thereof,
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises:
about 200 mg of the compound of Formula Ia or Ib, or a pharmaceutically acceptable salt thereof;
about 135 mg of mannitol;
about 137.5 mg of microcrystalline cellulose;
about 20 mg of crospovidone; and
about 7.5 mg of magnesium stearate.

In some embodiments, the tablet provided herein comprises:
about 175 mg to about 225 mg of the compound of Formula Ia or Ib, or a pharmaceutically acceptable salt thereof;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the tablet provided herein comprises:
about 200 mg of the compound of Formula Ia or Ib, or a pharmaceutically acceptable salt thereof;
about 135 mg to about 137 mg of mannitol;
about 135 mg to about 137 mg of microcrystalline cellulose;
about 20 mg of crospovidone; and
about 7.5 mg of magnesium stearate.

In some embodiments, the tablet provided herein comprises:
about 200 mg of the compound of Formula Ia or Ib, or a pharmaceutically acceptable salt thereof;
about 136.2 mg of mannitol;
about 136.2 mg of microcrystalline cellulose;
about 20 mg of crospovidone; and
about 7.5 mg of magnesium stearate.

In some embodiments, the tablet provided herein comprises:
about 200 mg of the compound of Formula Ia or Ib, or a pharmaceutically acceptable salt thereof;
about 136.3 mg of mannitol;
about 136.3 mg of microcrystalline cellulose;
about 20 mg of crospovidone; and
about 7.5 mg of magnesium stearate.

In some embodiments, the tablet provided herein comprises:
about 200 mg of the compound of Formula Ia or Ib, or a pharmaceutically acceptable salt thereof;
about 136.25 mg of mannitol;
about 136.25 mg of microcrystalline cellulose;
about 20 mg of crospovidone; and
about 7.5 mg of magnesium stearate.

In some embodiments, the tablet provided herein comprises:
about 5 mg to about 500 mg of the compound of Formula Ia, or a pharmaceutically acceptable salt thereof;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises:
about 175 mg to about 225 mg of the compound of Formula Ia, or a pharmaceutically acceptable salt thereof;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises:
about 200 mg of the compound of Formula Ia, or a pharmaceutically acceptable salt thereof;
about 135 mg of mannitol;
about 137.5 mg of microcrystalline cellulose;
about 20 mg of crospovidone; and
about 7.5 mg of magnesium stearate.

In some embodiments, the tablet provided herein comprises:
about 175 mg to about 225 mg of the compound of Formula Ia, or a pharmaceutically acceptable salt thereof;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the tablet provided herein comprises:
about 200 mg of the compound of Formula Ia, or a pharmaceutically acceptable salt thereof;
about 135 mg to about 137 mg of mannitol;
about 135 mg to about 137 mg of microcrystalline cellulose;
about 20 mg of crospovidone; and
about 7.5 mg of magnesium stearate.

In some embodiments, the tablet provided herein comprises:
about 200 mg of the compound of Formula Ia, or a pharmaceutically acceptable salt thereof;
about 136.2 mg of mannitol;
about 136.2 mg of microcrystalline cellulose;
about 20 mg of crospovidone; and
about 7.5 mg of magnesium stearate.

In some embodiments, the tablet provided herein comprises:
about 200 mg of the compound of Formula Ia, or a pharmaceutically acceptable salt thereof;
about 136.3 mg of mannitol;
about 136.3 mg of microcrystalline cellulose;
about 20 mg of crospovidone; and
about 7.5 mg of magnesium stearate.

In some embodiments, the tablet provided herein comprises:
about 200 mg of the compound of Formula Ia, or a pharmaceutically acceptable salt thereof;
about 136.25 mg of mannitol;
about 136.25 mg of microcrystalline cellulose;
about 20 mg of crospovidone; and
about 7.5 mg of magnesium stearate.

In some embodiments, the tablet provided herein comprises:
about 5 mg to about 500 mg of the compound of Formula Ia;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises:
about 175 mg to about 225 mg of the compound of Formula Ia;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises:
about 200 mg of the compound of Formula Ia;
about 135 mg of mannitol;
about 137.5 mg of microcrystalline cellulose;
about 20 mg of crospovidone; and
about 7.5 mg of magnesium stearate.

In some embodiments, the tablet provided herein comprises:
about 175 mg to about 225 mg of the compound of Formula Ia;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the tablet provided herein comprises:
about 200 mg of the compound of Formula Ia;
about 135 mg to about 137 mg of mannitol;
about 135 mg to about 137 mg of microcrystalline cellulose;
about 20 mg of crospovidone; and
about 7.5 mg of magnesium stearate.

In some embodiments, the tablet provided herein comprises:
about 200 mg of the compound of Formula Ia;
about 136.2 mg of mannitol;
about 136.2 mg of microcrystalline cellulose;
about 20 mg of crospovidone; and
about 7.5 mg of magnesium stearate.

In some embodiments, the tablet provided herein comprises:
about 200 mg of the compound of Formula Ia;
about 136.3 mg of mannitol;
about 136.3 mg of microcrystalline cellulose;
about 20 mg of crospovidone; and
about 7.5 mg of magnesium stearate.

In some embodiments, the tablet provided herein comprises:
about 200 mg of the compound of Formula Ia;
about 136.25 mg of mannitol;
about 136.25 mg of microcrystalline cellulose;
about 20 mg of crospovidone; and
about 7.5 mg of magnesium stearate.

In some embodiments, the tablet provided herein comprises:
about 5 mg to about 500 mg of the compound of Formula Ib, or a pharmaceutically acceptable salt thereof;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises:
about 175 mg to about 225 mg of the compound of Formula Ib, or a pharmaceutically acceptable salt thereof,
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises:
about 200 mg of the compound of Formula Ib, or a pharmaceutically acceptable salt thereof;
about 135 mg of mannitol;
about 137.5 mg of microcrystalline cellulose;
about 20 mg of crospovidone; and
about 7.5 mg of magnesium stearate.

In some embodiments, the tablet provided herein comprises:
about 175 mg to about 225 mg of the compound of Formula Ib, or a pharmaceutically acceptable salt thereof;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the tablet provided herein comprises:
about 200 mg of the compound of Formula Ib, or a pharmaceutically acceptable salt thereof;
about 135 mg to about 137 mg of mannitol;
about 135 mg to about 137 mg of microcrystalline cellulose;
about 20 mg of crospovidone; and
about 7.5 mg of magnesium stearate.

In some embodiments, the tablet provided herein comprises:
about 200 mg of the compound of Formula Ib, or a pharmaceutically acceptable salt thereof;
about 136.2 mg of mannitol;
about 136.2 mg of microcrystalline cellulose;
about 20 mg of crospovidone; and
about 7.5 mg of magnesium stearate.

In some embodiments, the tablet provided herein comprises:
about 200 mg of the compound of Formula Ib, or a pharmaceutically acceptable salt thereof;
about 136.3 mg of mannitol;
about 136.3 mg of microcrystalline cellulose;
about 20 mg of crospovidone; and
about 7.5 mg of magnesium stearate.

In some embodiments, the tablet provided herein comprises:
about 200 mg of the compound of Formula Ib, or a pharmaceutically acceptable salt thereof;
about 136.25 mg of mannitol;
about 136.25 mg of microcrystalline cellulose;
about 20 mg of crospovidone; and
about 7.5 mg of magnesium stearate.

In some embodiments, the tablet provided herein comprises:
about 5 mg to about 500 mg of the compound of Formula Ib;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises:
about 175 mg to about 225 mg of the compound of Formula Ib;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises:
about 200 mg of the compound of Formula Ib;
about 135 mg of mannitol;
about 137.5 mg of microcrystalline cellulose;
about 20 mg of crospovidone; and
about 7.5 mg of magnesium stearate.

In some embodiments, the tablet provided herein comprises:
about 175 mg to about 225 mg of the compound of Formula Ib;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the tablet provided herein comprises:
about 200 mg of the compound of Formula Ib;
about 135 mg to about 137 mg of mannitol;
about 135 mg to about 137 mg of microcrystalline cellulose;
about 20 mg of crospovidone; and
about 7.5 mg of magnesium stearate.

In some embodiments, the tablet provided herein comprises:
about 200 mg of the compound of Formula Ib;
about 136.2 mg of mannitol;
about 136.2 mg of microcrystalline cellulose;
about 20 mg of crospovidone; and
about 7.5 mg of magnesium stearate.

In some embodiments, the tablet provided herein comprises:
about 200 mg of the compound of Formula Ib;
about 136.3 mg of mannitol;
about 136.3 mg of microcrystalline cellulose;
about 20 mg of crospovidone; and
about 7.5 mg of magnesium stearate.

In some embodiments, the tablet provided herein comprises:
about 200 mg of the compound of Formula Ib;
about 136.25 mg of mannitol;
about 136.25 mg of microcrystalline cellulose;
about 20 mg of crospovidone; and
about 7.5 mg of magnesium stearate.

In some embodiments, the tablet provided herein comprises:
about 5 mg to about 500 mg of a crystalline form of the compound of Formula Ib;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises:
about 175 mg to about 225 mg of a crystalline form of the compound of Formula Ib;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises:
about 200 mg of a crystalline form of the compound of Formula Ib;
about 135 mg of mannitol;
about 137.5 mg of microcrystalline cellulose;
about 20 mg of crospovidone; and
about 7.5 mg of magnesium stearate.

In some embodiments, the tablet provided herein comprises:
about 175 mg to about 225 mg of a crystalline form of the compound of Formula Ib;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the tablet provided herein comprises:
about 200 mg of a crystalline form of the compound of Formula Ib;
about 135 mg to about 137 mg of mannitol;
about 135 mg to about 137 mg of microcrystalline cellulose;
about 20 mg of crospovidone; and
about 7.5 mg of magnesium stearate.

In some embodiments, the tablet provided herein comprises:
about 200 mg of a crystalline form of the compound of Formula Ib;
about 136.2 mg of mannitol;
about 136.2 mg of microcrystalline cellulose;
about 20 mg of crospovidone; and
about 7.5 mg of magnesium stearate.

In some embodiments, the tablet provided herein comprises:
about 200 mg of a crystalline form of the compound of Formula Ib;
about 136.3 mg of mannitol;
about 136.3 mg of microcrystalline cellulose;
about 20 mg of crospovidone; and
about 7.5 mg of magnesium stearate.

In some embodiments, the tablet provided herein comprises:
about 200 mg of a crystalline form of the compound of Formula Ib;
about 136.25 mg of mannitol;
about 136.25 mg of microcrystalline cellulose;
about 20 mg of crospovidone; and
about 7.5 mg of magnesium stearate.

In some embodiments, the tablet provided herein comprises:
about 5 mg to about 500 mg of a compound of Formula Ib, crystalline Form I;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the tablet provided herein comprises:
about 175 mg to about 225 mg of a compound of Formula Ib, crystalline Form I;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the tablet provided herein comprises:
about 200 mg of a compound of Formula Ib, crystalline Form I;
about 135 mg of mannitol;
about 137.5 mg of microcrystalline cellulose;
about 20 mg of crospovidone; and
about 7.5 mg of magnesium stearate.

In some embodiments, the tablet provided herein comprises:
about 175 mg to about 225 mg of a compound of Formula Ib, crystalline Form I;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the tablet provided herein comprises:
about 200 mg of a compound of Formula Ib, crystalline Form I;
about 135 mg to about 137 mg of mannitol;
about 135 mg to about 137 mg of microcrystalline cellulose;
about 20 mg of crospovidone; and
about 7.5 mg of magnesium stearate.

In some embodiments, the tablet provided herein comprises:

about 200 mg of a compound of Formula Ib, crystalline Form I;
about 136.2 mg of mannitol;
about 136.2 mg of microcrystalline cellulose;
about 20 mg of crospovidone; and
about 7.5 mg of magnesium stearate.

In some embodiments, the tablet provided herein comprises:
about 200 mg of a compound of Formula Ib, crystalline Form I;
about 136.3 mg of mannitol;
about 136.3 mg of microcrystalline cellulose;
about 20 mg of crospovidone; and
about 7.5 mg of magnesium stearate.

In some embodiments, the tablet provided herein comprises:
about 200 mg of a compound of Formula Ib, crystalline Form I;
about 136.25 mg of mannitol;
about 136.25 mg of microcrystalline cellulose;
about 20 mg of crospovidone; and
about 7.5 mg of magnesium stearate.

In some embodiments, the tablet provided herein comprises:
about 275 mg to about 325 mg of the compound of Formula Ia or Ib, or a pharmaceutically acceptable salt thereof;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the tablet provided herein comprises:
about 300 mg of the compound of Formula Ia or Ib, or a pharmaceutically acceptable salt thereof;
about 202.5 mg of mannitol;
about 206.25 mg of microcrystalline cellulose;
about 30 mg of crospovidone; and
about 11.25 mg of magnesium stearate.

In some embodiments, the tablet provided herein comprises:
about 275 mg to about 325 mg of the compound of Formula Ia, or a pharmaceutically acceptable salt thereof;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the tablet provided herein comprises:
about 300 mg of the compound of Formula Ia, or a pharmaceutically acceptable salt thereof;
about 202.5 mg of mannitol;
about 206.25 mg of microcrystalline cellulose;
about 30 mg of crospovidone; and
about 11.25 mg of magnesium stearate.

In some embodiments, the tablet provided herein comprises:
about 275 mg to about 325 mg of the compound of Formula Ib, or a pharmaceutically acceptable salt thereof;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the tablet provided herein comprises:
about 300 mg of the compound of Formula Ib, or a pharmaceutically acceptable salt thereof;
about 202.5 mg of mannitol;
about 206.25 mg of microcrystalline cellulose;
about 30 mg of crospovidone; and
about 11.25 mg of magnesium stearate.

In some embodiments, the tablet provided herein comprises:
about 275 mg to about 325 mg of a crystalline form of the compound of Formula Ib;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the tablet provided herein comprises:
about 300 mg of a crystalline form of the compound of Formula Ib;
about 202.5 mg of mannitol;
about 206.25 mg of microcrystalline cellulose;
about 30 mg of crospovidone; and
about 11.25 mg of magnesium stearate.

In some embodiments, the tablet provided herein comprises:
about 275 mg to about 325 mg of a compound of Formula Ib, crystalline Form I;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the tablet provided herein comprises:
about 300 mg of a compound of Formula Ib, crystalline Form I;
about 202.5 mg of mannitol;
about 206.25 mg of microcrystalline cellulose;
about 30 mg of crospovidone; and
about 11.25 mg of magnesium stearate.

In some embodiments, the tablet provided herein comprises:
about 375 mg to about 425 mg of the compound of Formula Ia or Tb, or a pharmaceutically acceptable salt thereof,
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the tablet provided herein comprises:
about 400 mg of the compound of Formula Ia or Tb, or a pharmaceutically acceptable salt thereof;
about 270 mg of mannitol;
about 275 mg of microcrystalline cellulose;
about 40 mg of crospovidone; and
about 15 mg of magnesium stearate.

In some embodiments, the tablet provided herein comprises:
about 375 mg to about 425 mg of the compound of Formula Ia, or a pharmaceutically acceptable salt thereof;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the tablet provided herein comprises:
about 400 mg of the compound of Formula Ia, or a pharmaceutically acceptable salt thereof;
about 270 mg of mannitol;
about 275 mg of microcrystalline cellulose;
about 40 mg of crospovidone; and
about 15 mg of magnesium stearate.

In some embodiments, the tablet provided herein comprises:
about 375 mg to about 425 mg of the compound of Formula Ib, or a pharmaceutically acceptable salt thereof;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the tablet provided herein comprises:
about 400 mg of the compound of Formula Ib, or a pharmaceutically acceptable salt thereof;
about 270 mg of mannitol;
about 275 mg of microcrystalline cellulose;
about 40 mg of crospovidone; and
about 15 mg of magnesium stearate.

In some embodiments, the tablet provided herein comprises:
about 375 mg to about 425 mg of a crystalline form of a compound of Formula Ib;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the tablet provided herein comprises:
about 400 mg of a crystalline form of a compound of Formula Ib;
about 270 mg of mannitol;
about 275 mg of microcrystalline cellulose;
about 40 mg of crospovidone; and
about 15 mg of magnesium stearate.

In some embodiments, the tablet provided herein comprises:
about 375 mg to about 425 mg of a compound of Formula Ib, crystalline Form I;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the tablet provided herein comprises:
about 400 mg of a compound of Formula Ib, crystalline Form I;
about 270 mg of mannitol;
about 275 mg of microcrystalline cellulose;
about 40 mg of crospovidone; and
about 15 mg of magnesium stearate.

In some embodiments, the tablet provided herein comprises:
about 1000 mg to about 1500 mg of the compound of Formula Ia or Tb, or a pharmaceutically acceptable salt thereof;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the tablet provided herein comprises:
about 1100 mg to about 1300 mg of the compound of Formula Ia or Ib, or a pharmaceutically acceptable salt thereof;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the tablet provided herein comprises:
about 1200 mg of the compound of Formula Ia or Ib, or a pharmaceutically acceptable salt thereof;
about 124 mg of mannitol;
about 124 mg of microcrystalline cellulose;
about 128 mg of crospovidone;
about 24 mg of magnesium stearate.

In some embodiments, the tablet provided herein comprises:
about 1000 mg to about 1500 mg of the compound of Formula Ia, or a pharmaceutically acceptable salt thereof;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the tablet provided herein comprises:
about 1100 mg to about 1300 mg of the compound of Formula Ia, or a pharmaceutically acceptable salt thereof,
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the tablet provided herein comprises:
about 1200 mg of the compound of Formula Ia, or a pharmaceutically acceptable salt thereof;
about 124 mg of mannitol;
about 124 mg of microcrystalline cellulose;
about 128 mg of crospovidone;
about 24 mg of magnesium stearate.

In some embodiments, the tablet provided herein comprises:
about 1000 mg to about 1500 mg of the compound of Formula Ia;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the tablet provided herein comprises:
about 1100 mg to about 1300 mg of the compound of Formula Ia;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the tablet provided herein comprises:
about 1200 mg of the compound of Formula Ia;
about 124 mg of mannitol;
about 124 mg of microcrystalline cellulose;
about 128 mg of crospovidone;
about 24 mg of magnesium stearate.

In some embodiments, the tablet provided herein comprises:
about 1000 mg to about 1500 mg of the compound of Formula Ib, or a pharmaceutically acceptable salt thereof,
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the tablet provided herein comprises:
about 1100 mg to about 1300 mg of the compound of Formula Ib, or a pharmaceutically acceptable salt thereof;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the tablet provided herein comprises:
about 1200 mg of the compound of Formula Ib, or a pharmaceutically acceptable salt thereof;
about 124 mg of mannitol;
about 124 mg of microcrystalline cellulose;
about 128 mg of crospovidone;
about 24 mg of magnesium stearate.

In some embodiments, the tablet provided herein comprises:
about 1000 mg to about 1500 mg of the compound of Formula Ib;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the tablet provided herein comprises:
about 1100 mg to about 1300 mg of the compound of Formula Ib;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the tablet provided herein comprises:
about 1200 mg of the compound of Formula Ib;
about 124 mg of mannitol;
about 124 mg of microcrystalline cellulose;
about 128 mg of crospovidone;
about 24 mg of magnesium stearate.

In some embodiments, the tablet provided herein comprises:
about 1000 mg to about 1500 mg of a crystalline form of the compound of Formula Ib;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the tablet provided herein comprises:
about 1100 mg to about 1300 mg of a crystalline form of the compound of Formula Ib;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the tablet provided herein comprises:
about 1200 mg of a crystalline form of the compound of Formula Ib;
about 124 mg of mannitol;
about 124 mg of microcrystalline cellulose;
about 128 mg of crospovidone;
about 24 mg of magnesium stearate.

In some embodiments, the tablet provided herein comprises:
about 1000 mg to about 1500 mg of a compound of Formula Ib, crystalline Form I;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the tablet provided herein comprises:
about 1100 mg to about 1300 mg of a compound of Formula Ib, crystalline Form I;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the tablet provided herein comprises:
about 1200 mg of a compound of Formula Ib, crystalline Form I;
about 124 mg of mannitol;
about 124 mg of microcrystalline cellulose;
about 128 mg of crospovidone;
about 24 mg of magnesium stearate.

The tablets disclosed herein may be uncoated or coated (in which case they include an outer film coat). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water-soluble material included in the film coating of the tablets may include a single polymer material, it may also be formed using a mixture of more than one polymer. The coating may be white or colored.

Suitable coatings include, but are not limited to, polymeric film coatings such as those comprising polyvinyl alcohol e.g. ‘Opadry® II’ (which includes part-hydrolysed PVA, titanium dioxide, macrogol 3350 and talc, with optional colouring such as iron oxide or indigo carmine or iron oxide yellow or FD&C yellow #6). The amount of coating will generally be between about 1-8% of the uncoated tablet's weight. In some embodiments, the amount of coating will generally be between about 2-6% of the uncoated tablet's weight. In some embodiments, the amount of coating will generally be between about 3-4% of the uncoated tablet's weight.

In some embodiments, the amount of coating is 1% of the uncoated tablet's weight. In some embodiments, the amount of coating is 2% of the uncoated tablet's weight. In some embodiments, the amount of coating is 3% of the uncoated tablet's weight. In some embodiments, the amount of coating is 4% of the uncoated tablet's weight. In some embodiments, the amount of coating is 5% of the uncoated tablet's weight. In some embodiments, the amount of coating is 6% of the uncoated tablet's weight. In some embodiments, the amount of coating is 7% of the uncoated tablet's weight. In some embodiments, the amount of coating is 8% of the uncoated tablet's weight.

In some embodiments, the coating is Opadry II Purple 85F140073. Opadry II Purple 85F140073 contains 40.00% w/w polyvinyl alcohol (USP/Ph. Eur.), 24.20% w/w titanium dioxide (USP/Ph. Eur.), 20.20% w/w macrogol/polyethylene glycol (USP/Ph. Eur.), 14.80% w/w talc (USP/Ph. Eur.), 0.58% w/w iron oxide red (NF), and 0.22% w/w ferrosoferric oxide/black iron oxide (NF).

In some embodiments, the coating is Opadry II Gray 85F97517. Opadry II Gray 85F97517 contains 40.00% w/w polyvinyl alcohol (USP/Ph.Eur.), 24.74% w/w titanium dioxide, (USP/Ph.Eur.), 20.20% w/w macrogol/polyethylene glycol (NF/Ph.Eur.), 14.80% (w/w) talc (USP/Ph.Eur.), and 0.26% w/w ferrosoferric oxide/black iron oxide (NF). In some embodiments, the coating is Opadry TF Gray 273F17005. Opadry TF Gray 273F175005 contains 37.000% w/w polyvinyl alcohol (USP/FCC/PhEur/JPE/ChP/GB), 34.850% w/w calcium carbonate (USP/FCC/PhEur/JP/JECFA/JSFA/GB), 14.500% w/w microcrystalline cellulose (NF/PhEur/JP/ChP), 7.000% w/w macrogol 6000 JP/PEG (USP/FCC/PhEur/JECFA/JP), 5.000% w/w magnesium aluminometasilicate (Type IA NF/Type A PhEur/JP), 1.000% carnauba wax (NF/FCC/PhEur/JP/ChP/GB), 0.500% xanthan gum (NF/FCC/PhEur/JPE/ChP/JECFA), 0.150% w/w ferrosoferric oxide (NF)/black iron oxide (JPE/JECFA/ChP).

### Methods of Use

In some embodiments, the methods provided herein comprise treating or preventing a human immunodeficiency virus (HIV) infection in the patient. In some embodiments, the methods provided herein comprise treating a HIV infection in the patient. In some embodiments, the methods provided herein comprise preventing a HIV infection in the patient. In some embodiments, the patient may have or be at risk of contracting an HIV infection. In some embodiments, the patient has been identified as an individual who is at risk of sexual transmission of HIV. In some embodiments, the individual has been identified as a man (e.g., who has sexual intercourse with a man or a woman), transgender man, transgender woman, a woman (e.g., who has sexual intercourse with a man or a woman), as a gender non-binary individual (e.g., who has sexual intercourse with a man or a woman), and/or a sex worker.

As used herein, an “at risk” individual is an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). For example, individuals at risk for AIDS are those having HIV.

In some embodiments, the individual has been identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual has been identified as having sex with partners of unknown HIV status.

In some embodiments, the individual has been identified as having sex with people who are living with HIV but not on HIV treatment and with an undetectable viral load.

In some embodiments, the individual has been identified as having sex in a geographic area where HIV is common.

In some embodiments, the individual has been identified as having sex in a geographic area of North America (e.g., United States, Canada, Mexico) where HIV is common. In some embodiments, the individual has been identified as having sex in a geographic area of the United States where HIV is common. In some embodiments, the individual has been identified as having sex in a geographic area of the Canada where HIV is common. In some embodiments, the individual has been identified as having sex in a geographic area of the Mexico where HIV is common.

In some embodiments, the individual has been identified as having sex in a geographic area of Africa where HIV is common. In some embodiments, the individual has been identified as having sex in a geographic area of South Africa where HIV is common. In some embodiments, the individual has been identified as having sex in a geographic area of Uganda where HIV is common.

In some embodiments, the individual has been identified as having sex in a geographic area of Europe where HIV is common.

In some embodiments, the individual has been identified as having sex in a geographic area of Asia (e.g., Thailand) where HIV is common. In some embodiments, the individual has been identified as having sex in a geographic area of Thailand where HIV is common.

In some embodiments, the individual has been identified as having sex in a geographic area of South America (e.g., Argentina, Brazil, Peru, and the like) where HIV is common. In some embodiments, the individual has been identified as having sex in a geographic area of Argentina where HIV is common. In some embodiments, the individual has been identified as having sex in a geographic area of Brazil where HIV is common. In some embodiments, the individual has been identified as having sex in a geographic area of Peru where HIV is common.

In some embodiments, the individual has been identified as having sex while under the influence of substances or alcohol.

In some embodiments, the individual has been identified as not using condoms consistently with partners of unknown status.

In some embodiments, the individual has been identified an individual who injects drugs.

In some embodiments, the individual is a cisgender woman. In some embodiments, the individual is an adolescent cisgender woman. In some embodiments, the adolescent cisgender woman is about 16 to about 25 years of age, for example, about 16, about 17, about 18, about 19, about 20, about 21, about 22, about 23, about 24, about 25, or about 26 years of age.

In some embodiments, the individual is a cisgender woman identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a cisgender woman identified as having sex with partners of unknown HIV status.

In some embodiments, the individual is a cisgender woman identified as having sex with people who are living with HIV but not on HIV treatment and with an undetectable viral load.

In some embodiments, the individual is a cisgender woman identified as having sex in a geographic area where HIV is common.

In some embodiments, the individual is a cisgender woman identified as having sex in a geographic area of North America (e.g., United States, Canada, Mexico) where HIV is common. In some embodiments, the individual is a cisgender woman identified as having sex in a geographic area of the United States where HIV is common. In some embodiments, the individual is a cisgender woman identified as having sex in a geographic area of the Canada where HIV is common. In some embodiments, the individual is a cisgender woman identified as having sex in a geographic area of the Mexico where HIV is common.

In some embodiments, the individual is a cisgender woman identified as having sex in a geographic area of Africa where HIV is common. In some embodiments, the individual is a cisgender woman identified as having sex in a geographic area of South Africa where HIV is common. In some embodiments, the individual is a cisgender woman identified as having sex in a geographic area of Uganda where HIV is common.

In some embodiments, the individual is a cisgender woman identified as having sex in a geographic area of Europe where HIV is common.

In some embodiments, the individual is a cisgender woman identified as having sex in a geographic area of Asia (e.g., Thailand) where HIV is common. In some embodiments, the individual is a cisgender woman identified as having sex in a geographic area of Thailand where HIV is common.

In some embodiments, the individual is a cisgender woman identified as having sex in a geographic area of South America (e.g., Argentina, Brazil, Peru, and the like) where HIV is common. In some embodiments, the individual is a cisgender woman identified as having sex in a geographic area of Argentina where HIV is common. In some embodiments, the individual is a cisgender woman identified as having sex in a geographic area of Brazil where HIV is common. In some embodiments, the individual is a cisgender woman identified as having sex in a geographic area of Peru where HIV is common.

In some embodiments, the individual is a cisgender woman identified as having sex while under the influence of substances or alcohol.

In some embodiments, the individual is a cisgender woman identified as not using condoms consistently with partners of unknown status.

In some embodiments, the individual is a cisgender woman identified as an individual who injects drugs.

In some embodiments, the individual is a transgender woman. In some embodiments, the individual is an adolescent transgender woman. In some embodiments, the adolescent transgender woman is about 16 to about 25 years of age, for example, about 16, about 17, about 18, about 19, about 20, about 21, about 22, about 23, about 24, about 25, or about 26 years of age.

In some embodiments, the individual is a transgender woman identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a transgender woman identified as having sex with partners of unknown HIV status.

In some embodiments, the individual is a transgender woman identified as having sex with people who are living with HIV but not on HIV treatment and with an undetectable viral load.

In some embodiments, the individual is a transgender woman identified as having sex in a geographic area where HIV is common.

In some embodiments, the individual is a transgender woman identified as having sex in a geographic area of North America (e.g., United States, Canada, Mexico) where HIV is common. In some embodiments, the individual is a transgender woman identified as having sex in a geographic area of the United States where HIV is common. In some embodiments, the individual is a transgender woman identified as having sex in a geographic area of the Canada where HIV is common. In some embodiments, the individual is a transgender woman identified as having sex in a geographic area of the Mexico where HIV is common.

In some embodiments, the individual is a transgender woman identified as having sex in a geographic area of Africa where HIV is common. In some embodiments, the individual is a transgender woman identified as having sex in a geographic area of South Africa where HIV is common. In some embodiments, the individual is a transgender woman identified as having sex in a geographic area of Uganda where HIV is common.

In some embodiments, the individual is a transgender woman identified as having sex in a geographic area of Europe where HIV is common.

In some embodiments, the individual is a transgender woman identified as having sex in a geographic area of Asia (e.g., Thailand) where HIV is common. In some embodiments, the individual is a transgender woman identified as having sex in a geographic area of Thailand where HIV is common.

In some embodiments, the individual is a transgender woman identified as having sex in a geographic area of South America (e.g., Argentina, Brazil, Peru, and the like) where HIV is common. In some embodiments, the individual is a transgender woman identified as having sex in a geographic area of Argentina where HIV is common. In some embodiments, the individual is a transgender woman identified as having sex in a geographic area of Brazil where HIV is common. In some embodiments, the individual is a transgender woman identified as having sex in a geographic area of Peru where HIV is common.

In some embodiments, the individual is a transgender woman identified as having sex while under the influence of substances or alcohol.

In some embodiments, the individual is a transgender woman identified as not using condoms consistently with partners of unknown status.

In some embodiments, the individual is a transgender woman identified as an individual who injects drugs.

In some embodiments, the individual is a cisgender man. In some embodiments, the individual is an adolescent cisgender man. In some embodiments, the adolescent cisgender man is about 16 to about 25 years of age, for example, about 16, about 17, about 18, about 19, about 20, about 21, about 22, about 23, about 24, about 25, or about 26 years of age.

In some embodiments, the individual is a cisgender man identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a cisgender man identified as having sex with partners of unknown HIV status.

In some embodiments, the individual is a cisgender man identified as having sex with people who are living with HIV but not on HIV treatment and with an undetectable viral load.

In some embodiments, the individual is a cisgender man identified as having sex in a geographic area where HIV is common.

In some embodiments, the individual is a cisgender man identified as having sex in a geographic area of North America (e.g., United States, Canada, Mexico) where HIV is common. In some embodiments, the individual is a cisgender man identified as having sex in a geographic area of the United States where HIV is common. In some embodiments, the individual is a cisgender man identified as having sex in a geographic area of the Canada where HIV is common. In some embodiments, the individual is a cisgender man identified as having sex in a geographic area of the Mexico where HIV is common.

In some embodiments, the individual is a cisgender man identified as having sex in a geographic area of Africa where HIV is common. In some embodiments, the individual is a cisgender man identified as having sex in a geographic area of South Africa where HIV is common. In some embodiments, the individual is a cisgender man identified as having sex in a geographic area of Uganda where HIV is common.

In some embodiments, the individual is a cisgender man identified as having sex in a geographic area of Europe where HIV is common.

In some embodiments, the individual is a cisgender man identified as having sex in a geographic area of Asia (e.g., Thailand) where HIV is common. In some embodiments, the individual is a cisgender man identified as having sex in a geographic area of Thailand where HIV is common.

In some embodiments, the individual is a cisgender man identified as having sex in a geographic area of South America (e.g., Argentina, Brazil, Peru, and the like) where HIV is common. In some embodiments, the individual is a cisgender man identified as having sex in a geographic area of Argentina where HIV is common. In some embodiments, the individual is a cisgender man identified as having sex in a geographic area of Brazil where HIV is common. In some embodiments, the individual is a cisgender man identified as having sex in a geographic area of Peru where HIV is common.

In some embodiments, the individual is a cisgender man identified as having sex while under the influence of substances or alcohol.

In some embodiments, the individual is a cisgender man identified as not using condoms consistently with partners of unknown status.

In some embodiments, the individual is a cisgender man identified as an individual who injects drugs.

In some embodiments, the individual is a transgender man. In some embodiments, the individual is an adolescent transgender man. In some embodiments, the adolescent transgender man is about 16 to about 25 years of age, for example, about 16, about 17, about 18, about 19, about 20, about 21, about 22, about 23, about 24, about 25, or about 26 years of age.

In some embodiments, the individual is a transgender man identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a transgender man identified as having sex with partners of unknown HIV status.

In some embodiments, the individual is a transgender man identified as having sex with people who are living with HIV but not on HIV treatment and with an undetectable viral load.

In some embodiments, the individual is a transgender man identified as having sex in a geographic area where HIV is common.

In some embodiments, the individual is a transgender man identified as having sex in a geographic area of North America (e.g., United States, Canada, Mexico) where HIV is common. In some embodiments, the individual is a transgender man identified as having sex in a geographic area of the United States where HIV is common. In some embodiments, the individual is a transgender man identified as having sex in a geographic area of the Canada where HIV is common. In some embodiments, the individual is a transgender man identified as having sex in a geographic area of the Mexico where HIV is common.

In some embodiments, the individual is a transgender man identified as having sex in a geographic area of Africa where HIV is common. In some embodiments, the individual is a transgender man identified as having sex in a geographic area of South Africa where HIV is common. In some embodiments, the individual is a transgender man identified as having sex in a geographic area of Uganda where HIV is common.

In some embodiments, the individual is a transgender man identified as having sex in a geographic area of Europe where HIV is common.

In some embodiments, the individual is a transgender man identified as having sex in a geographic area of Asia (e.g., Thailand) where HIV is common. In some embodiments, the individual is a transgender man identified as having sex in a geographic area of Thailand where HIV is common.

In some embodiments, the individual is a transgender man identified as having sex in a geographic area of South America (e.g., Argentina, Brazil, Peru, and the like) where HIV is common. In some embodiments, the individual is a transgender man identified as having sex in a geographic area of Argentina where HIV is common. In some embodiments, the individual is a transgender man identified as having sex in a geographic area of Brazil where HIV is common. In some embodiments, the individual is a transgender man identified as having sex in a geographic area of Peru where HIV is common.

In some embodiments, the individual is a transgender man identified as having sex while under the influence of substances or alcohol.

In some embodiments, the individual is a transgender man identified as not using condoms consistently with partners of unknown status.

In some embodiments, the individual is a transgender man identified as an individual who injects drugs.

In some embodiments, the individual is a gender non-binary individual. In some embodiments, the individual is an adolescent gender non-binary individual. In some embodiments, the adolescent gender non-binary individual is about 16 to about 25 years of age, for example, about 16, about 17, about 18, about 19, about 20, about 21, about 22, about 23, about 24, about 25, or about 26 years of age.

In some embodiments, the individual is a gender non-binary individual identified as one or more of the following:
having sex with partners of unknown HIV status;
having sex with people who are living with HIV but not on HIV treatment and with an undetectable viral load;
having sex in a geographic area where HIV is common;
having sex while under the influence of substances or alcohol;
not using condoms consistently with partners of unknown status; and
an individual who injects drugs, including, for example, but not limited to people who inject opioids, stimulants, psychoactive drugs, or a combination of any of the foregoing. Non-limiting examples of opioids include fentanyl and heroin. Non-limiting examples of stimulants include cocaine and amphetamines. Non-limiting examples of psychoactive drugs include benzodiazepines.

In some embodiments, the individual is a gender non-binary individual identified as having sex with partners of unknown HIV status.

In some embodiments, the individual is a gender non-binary individual identified as having sex with people who are living with HIV but not on HIV treatment and with an undetectable viral load.

In some embodiments, the individual is a gender non-binary individual identified as having sex in a geographic area where HIV is common.

In some embodiments, the individual is a gender non-binary individual identified as having sex in a geographic area of North America (e.g., United States, Canada, Mexico) where HIV is common. In some embodiments, the individual is a gender non-binary individual identified as having sex in a geographic area of the United States where HIV is common. In some embodiments, the individual is a gender non-binary individual identified as having sex in a geographic area of the Canada where HIV is common. In some embodiments, the individual is a gender non-binary individual identified as having sex in a geographic area of the Mexico where HIV is common.

In some embodiments, the individual is a gender non-binary individual identified as having sex in a geographic area of Africa where HIV is common. In some embodiments, the individual is a gender non-binary individual identified as having sex in a geographic area of South Africa where HIV is common. In some embodiments, the individual is a gender non-binary individual identified as having sex in a geographic area of Uganda where HIV is common.

In some embodiments, the individual is a gender non-binary individual identified as having sex in a geographic area of Europe where HIV is common.

In some embodiments, the individual is a gender non-binary individual identified as having sex in a geographic area of Asia (e.g., Thailand) where HIV is common. In some embodiments, the individual is a gender non-binary individual identified as having sex in a geographic area of Thailand where HIV is common.

In some embodiments, the individual is a gender non-binary individual identified as having sex in a geographic area of South America (e.g., Argentina, Brazil, Peru, and the like) where HIV is common. In some embodiments, the individual is a gender non-binary individual identified as having sex in a geographic area of Argentina where HIV is common. In some embodiments, the individual is a gender non-binary individual identified as having sex in a geographic area of Brazil where HIV is common. In some embodiments, the individual is a gender non-binary individual identified as having sex in a geographic area of Peru where HIV is common.

In some embodiments, the individual is a gender non-binary individual identified as having sex while under the influence of substances or alcohol.

In some embodiments, the individual is a gender non-binary individual identified as not using condoms consistently with partners of unknown status.

In some embodiments, the individual is a gender non-binary individual identified as an individual who injects drugs.

In some embodiments, the individual has been identified as:
having anal sex with at least two different sexual partners and no consistent condom use over the last 6 months; and/or
having history of sexually transmitted diseases (STDs) during the last 12 months (e.g., syphilis, gonorrhea, chlamydiae, HBV or HCV infection); and/or
using psycho-active drugs during sexual intercourses (e.g., cocaine, gammahydroxybutyric acid (GHB), methylenedioxymethamphetamine (MDMA), mephedrone); and/or
having sexual intercourse with one or more partners originating from a region with high prevalence of HIV infection (>1%) (e.g., South America, Sub-Saharan Africa, South-East Asia, Eastern Europe, French Guyana) and no consistent condom use; and/or
a sex worker; and/or
having a sexual partner who is an intravenous drug user sharing injection material; and/or
having an HIV-infected sexual partner with a detectable plasma viral load (e.g., >50 copies (cp)/milliliter (mL)); and/or
a cisgender man;
a transgender man;
a cisgender woman;
a transgender woman;
a gender non-binary individual; and/or
a person who injects drugs, including, for example, but not limited to people who inject opioids, stimulants, psychoactive drugs, or a combination of any of the foregoing. Non-limiting examples of opioids include fentanyl and heroin.

Non-limiting examples of stimulants include cocaine and amphetamines.

Non-limiting examples of psychoactive drugs include benzodiazepines.

In some embodiments, the patient is HIV-negative. In some embodiments, the HIV is HIV-1. In some embodiments, the HIV is HIV-2. In some embodiments, the HIV is HIV-1 and HIV-2.

As used herein, “HIV” or “Human Immunodeficiency Virus” refers to HIV-1 and/or to HIV-2.

In some embodiments, the patient is HIV-negative. In some embodiments, the patient is HIV-1 negative. In some embodiments, the patient is HIV-2 negative. In some embodiments, the patient is HIV-1 and HIV-2 negative.

In some embodiments, the patient has tested HIV-negative. In some embodiments, the patient has tested HIV-1 negative. In some embodiments, the patient has tested HIV-2 negative. In some embodiments, the patient has tested HIV-1 and HIV-2 negative.

In some embodiments, the patient has been identified as testing negative for HIV. In some embodiments, the patient has been identified as testing negative for HIV-1. In some embodiments, the patient has been identified as testing negative for HIV-2. In some embodiments, the patient has been identified as testing negative for HIV-1 and HIV-2.

In some embodiments, the method provided herein further comprises identifying the patient as testing negative for HIV. In some embodiments, the method provided herein further comprises identifying the patient as testing negative for HIV-1. In some embodiments, the method provided herein further comprises identifying the patient as testing negative for HIV-2. In some embodiments, the method provided herein further comprises identifying the patient as testing negative for HIV-1 and HIV-2.

In some embodiments, the method provided herein further comprises testing the patient for HIV prior to administration of each administration of a composition provided herein (e.g., a tablet comprising a compound of Formula Ia or Ib, or a pharmaceutically acceptable salt or free acid form thereof). In some embodiments, the method provided herein further comprises testing the patient for HIV-1 prior to each administration of a composition provided herein (e.g., a tablet comprising a compound of Formula Ia or Ib, or a pharmaceutically acceptable salt or free acid form thereof). In some embodiments, the method provided herein further comprises testing the patient for HIV-2 prior to each administration of a composition provided herein (e.g., a tablet comprising a compound of Formula Ia or Ib, or a pharmaceutically acceptable salt or free acid form thereof). In some embodiments, the method provided herein further comprises testing the patient for HIV-1 and HIV-2 prior to each administration of a composition provided herein (e.g., a tablet comprising a compound of Formula Ia or Ib, or a pharmaceutically acceptable salt or free acid form thereof).

In some embodiments, the method provided herein comprises obtaining a negative test result for HIV of the patient, prior to each administration of a composition provided herein (e.g., a tablet comprising a compound of Formula Ia or Ib, or a pharmaceutically acceptable salt or free acid form thereof). In some embodiments, the method provided herein comprises obtaining a negative test result of the patient for HIV-1, prior to each administration of a composition provided herein (e.g., a tablet comprising a compound of Formula Ia or Ib, or a pharmaceutically acceptable salt or free acid form thereof). In some embodiments, the method provided herein comprises obtaining a negative test result of the patient for HIV-2, prior to each administration of a composition provided herein (e.g., a tablet comprising a compound of Formula Ia or Ib, or a pharmaceutically acceptable salt or free acid form thereof). In some embodiments, the method provided herein comprises obtaining a negative test result of the patient for HIV-1 and HIV-2, prior to each administration of a composition provided herein (e.g., a tablet comprising a compound of Formula Ia or Ib, or a pharmaceutically acceptable salt or free acid form thereof).

The term “patient” is meant to refer to a human who is in need of therapeutic or preventative treatment for a viral infection, such as HIV infection.

As used herein, the terms “prevention” or “preventing” refer to the administration of a compound, or a pharmaceutically acceptable salt or free acid form thereof, or composition comprising the compound, pharmaceutically acceptable salt, or free acid form thereof according to the present disclosure pre- or post-exposure of the patient to the virus but before the appearance of symptoms of the disease, and/or prior to the detection of the virus in the blood. The terms also refer to prevention of the appearance of symptoms of the disease and/or to prevent the virus from reaching detectable levels in the blood. The terms include both pre-exposure prophylaxis (PrEP), as well as post-exposure prophylaxis (PEP) and event driven or “on demand” prophylaxis. The terms also refer to prevention of perinatal transmission of HIV from mother to baby by administration of a compound, pharmaceutically acceptable salt thereof, or composition comprising the compound or the pharmaceutically acceptable salt thereof according to the present disclosure to the mother before giving birth and to the child within the first days of life. The term also refers to prevention of transmission of HIV through blood transfusion.

As used herein, the term “period of exposure” refers to a period of time, ranging from a single event or to multiple events over an extended period of time, in which a patient is exposed to HIV. For example, a patient who engages in one sexual intercourse event with a partner who is HIV-positive has a period of exposure that is limited to the time and duration of that one sexual intercourse event with that partner. As another example, a patient who has sexual intercourse with a partner who is HIV-positive on multiple occasions over an extended period of time (e.g., days, weeks, months, or years) has a period of exposure that ranges from the first instance to the last instance of sexual intercourse with that partner.

In some embodiments, the methods disclosed herein may comprise event driven administration of a composition provided herein (e.g., a composition comprising a compound of Formula Ia or Ib, or a pharmaceutically acceptable salt or free acid form thereof), to the patient. As used herein, the terms “event driven” or “event driven administration” refer to administration of a composition provided herein, (1) prior to an event (e.g., 2 hours, 1 day, 2 days, 5 days, 7 days, 10 days, 14 days, 28 days (i.e., one month), or more days prior to the event) that would expose the patient to HIV (or that would otherwise increase the patient's risk of acquiring HIV); and/or (2) during an event (or more than one recurring event) that would expose the patient to HIV (or that would otherwise increase the patient's risk of acquiring HIV); and/or (3) after an event (or after the final event in a series of recurring events) that would expose the patient to HIV (or that would otherwise increase the patient's risk of acquiring HIV). In some embodiments, the event driven administration is performed pre-exposure of the patient to the HIV. In some embodiments, the event driven administration is performed during exposure of the patient to the HIV. In some embodiments, the event driven administration is performed post-exposure of the patient to the HIV.

In some embodiments, the event driven administration is performed pre-exposure of the patient to the HIV and during exposure of the patient to the HIV.

In some embodiments, the event driven administration is performed pre-exposure of the patient to the HIV and post-exposure of the patient to the HIV.

In some embodiments, the event driven administration is performed during exposure of the patient to the HIV and post-exposure of the patient to the HIV.

In certain embodiments, the methods disclosed herein involve administration prior to and/or after an event that would expose the patient to HIV or that would otherwise increase the patient's risk of acquiring HIV, e.g., as pre-exposure prophylaxis (PrEP) and/or as post-exposure prophylaxis (PEP). Examples of events that could increase a patient's risk of acquiring HIV include, without limitation, no condom use during anal intercourse with an HIV positive partner or a partner of unknown HIV status; anal intercourse with more than 3 sexual partners; exchange of money, gifts, shelter or drugs for anal sex; sex with male partner and diagnosis of sexually transmitted infection; and no consistent use of condoms with a sexual partner known to be HIV positive. In some embodiments, the methods disclosed herein comprise pre-exposure prophylaxis (PrEP). In some embodiments, methods disclosed herein comprise post-exposure prophylaxis (PEP). In some embodiments, the methods disclosed herein comprise pre-exposure prophylaxis (PrEP) and post-exposure prophylaxis (PEP).

In some embodiments, a composition provided herein is administered before exposure of the patient to the HIV.

In some embodiments, a composition provided herein is administered during the period of exposure of the patient to the HIV.

In some embodiments, a composition provided herein is administered after final exposure of the patient to the HIV.

In some embodiments, a composition provided herein is administered before and during exposure of the patient to the HIV.

In some embodiments, a composition provided herein is administered before and after exposure of the patient to the HIV.

In some embodiments, a composition provided herein is administered during and after exposure of the patient to the HIV.

In some embodiments, a composition provided herein is administered before, during, and after exposure of the patient to the HIV.

In some embodiments, the dose of the composition administered during each period (i.e., before, during, and after exposure) may be different, i.e, independently selected from any of the doses disclosed herein.

In certain embodiments, e.g., when administered as PrEP, a composition provided herein is administered 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity) prior to an event that would increase the patient's risk of acquiring HIV (e.g., prior to sexual intercourse or other exposure to the HIV). In some embodiments, a composition provided herein is administered within 14 days, 13 days, 12 days, 11 days, 10 days, 9 days, 8 days, 7 days, 6 days, 5 days, 4 days, 3 days, 2 days, or 1 day prior to an event that would increase the patient's risk of acquiring HIV (e.g., prior to sexual intercourse or other exposure to the HIV). In some embodiments, a composition provided herein is administered within 72 hours, 60 hours, 48 hours, 24 hours, 12 hours, 9 hours, 6 hours, 4 hours, 3 hours, 2 hours, or 1 hour prior to an event that would increase the patient's risk of acquiring HIV (e.g., prior to sexual intercourse or other exposure to the HIV). In certain embodiments, when a composition provided herein is administered prior to an event that would increase the patient's risk of acquiring HIV, it is administered daily prior to the event (e.g., sexual activity). In certain embodiments, when a composition provided herein is administered prior to an event that would increase the patient's risk of acquiring HIV, it is administered one to three times prior to the event. In certain embodiments, when a composition provided herein is administered prior to an event that would increase the patient's risk of acquiring HIV, it is administered one time (i.e., once) prior to the event.

In some embodiments, a composition provided herein is administered from about 14 days to about one day before exposure of the patient to the HIV. In some embodiments, a composition provided herein is administered once from about 14 days to about one day before exposure of the patient to the HIV.

In some embodiments, a composition provided herein is administered from about 10 days to about 5 days before exposure of the patient to the HIV. In some embodiments, a composition provided herein is administered once from about 10 days to about 5 days before exposure of the patient to the HIV.

In some embodiments, a composition provided herein is administered from about 8 days to about 6 days before exposure of the patient to the HIV. In some embodiments, a composition provided herein is administered once from about 8 days to about 6 days before exposure of the patient to the HIV.

In some embodiments, a composition provided herein is administered about 7 days before exposure of the patient to the HIV. In some embodiments, a composition provided herein is administered once about 7 days before exposure of the patient to the HIV.

In some embodiments, a composition provided herein is administered from about 72 hours to about 1 hour before exposure of the patient to the HIV. In some embodiments, a composition provided herein is administered once from about 72 hours to about 1 hour before exposure of the patient to the HIV.

In some embodiments of the methods provided herein, the pre-exposure prophylaxis (PrEP) comprises continuous PrEP.

In certain embodiments where a composition provided herein is administered before exposure of the patient to the HIV, the methods disclosed herein further comprise administering one or more additional doses of the compound of Formula Ia or Ib, or a pharmaceutically acceptable salt or free acid form thereof, during, and/or after exposure of the patient to the HIV.

In some embodiments, e.g., when administered as part of a PrEP regimen or as part of a PEP regimen, a composition provided herein is administered during the period of exposure of the patient to the HIV. In certain embodiments wherein a composition provided herein is administered before HIV exposure, the composition is administered about every 7 days, about every 14 days, about every 21 days, about every 28 days, about every 35 days, about every 42 days, or about every 6 months, or about every 12 months (e.g., as a single dose) during the time of HIV exposure (e.g., during the time period of sexual activity with a sexual partner known to be HIV positive). In some embodiments, a composition provided herein is administered once about every 7 days, about every 14 days, about every 21 days, about every 28 days, about every 35 days, about every 42 days, about every 6 months, or about every 12 months during the period of exposure of the patient to the HIV.

In some embodiments, a composition provided herein administered prior to exposure to the HIV is at a different dose than a composition (e.g., a composition comprising a compound of Formula Ia or Ib, or a pharmaceutically acceptable salt or free acid form thereof) administered during and/or after exposure to the HIV. For example, in some embodiments, the dose of the compound of Formula Ia or Ib, or a pharmaceutically acceptable salt or free acid form thereof, is increased, e.g., as a double dose, as a triple dose, and the like as compared to an earlier administered dose (e.g., a dose prior to exposure to the HIV). In some embodiments, the increased dose of the compound of Formula Ia or Ib, or a pharmaceutically acceptable salt or free acid form thereof, is a double dose. In some embodiments, the dose of the compound of Formula Ia or Ib, or a pharmaceutically acceptable salt or free acid form thereof, is decreased, e.g., a half dose as compared to an earlier administered dose (e.g., a dose prior to exposure to the HIV).

In some embodiments, a composition provided herein (e.g., a composition comprising a compound of Formula Ia or Ib, or a pharmaceutically acceptable salt or free acid form thereof) is administered as a single dose from about 1 hour to about 10 days before exposure of the patient to the HIV.

Additional examples of PrEP and/or PEP can be found, for example, at the clinical trial summary titled “On Demand Antiretroviral Pre-exposure Prophylaxis for HIV Infection in Men Who Have Sex With Men” (Clinical Trial #NCT01473472); the clinical trial summary titled “Prevention of HIV in Île-de-France” (Clinical Trials #NCT03113123), and at Molina et al, N. Engl. J. Med. 2015, 353:2237-2246, the disclosure of each of which is incorporated herein by reference in its entirety.

In some embodiments, e.g., when administered as part of a PrEP regimen or as part of a PEP regimen, a composition provided herein is administered 1 hour to 10 days, 1 hour to 7 days, 1 hour to 5 days, 1 to 72 hours, 1 to 48 hours, 1 to 36 hours, 1 to 24 hours, or 1 to 12 hours following an event that would increase the patient's risk of acquiring HIV (e.g., following sexual intercourse or other exposure to the HIV).

In certain embodiments, e.g., when administered as PEP, a composition provided herein is administered for 7 days, 14 days, 21 days, 28 days, 30 days, or 45 days following an event that would increase the patient's risk of acquiring HIV (e.g., following sexual intercourse or other exposure to the HIV). In certain embodiments, e.g., when administered as PEP, a composition provided herein is administered for 30 days following an event that would increase the patient's risk of acquiring HIV (e.g., following sexual intercourse or other exposure to the HIV). In certain embodiments, a composition provided herein is administered less than 1 hour, 2 hours, 3 hours, 4 hours, 5 hours, 6 hours, 7 hours, 8 hours, 9 hours, 12 hours, 18 hours, 24 hours, 36 hours, or 48 hours following an event that would increase the patient's risk of acquiring HIV (e.g., following sexual intercourse or other exposure to the HIV virus). In certain embodiments, a composition provided herein is administered for 1 day, 2 days, 3 days, 4 days, or 5 days following an event that would increase the patient's risk of acquiring HIV (e.g., following sexual intercourse or other exposure to the HIV). In certain embodiments, when a composition provided herein is administered following an event that would increase the patient's risk of acquiring HIV, it is administered daily following the event. In certain embodiments, when a composition provided herein is administered following an event that would increase the patient's risk of acquiring HIV, it is administered one to three times following the event. In certain embodiments, when a composition provided herein is administered following an event that would increase the patient's risk of acquiring HIV, it is administered once following the event.

In certain embodiments, e.g., when administered as PEP, a composition provided herein is administered once about every week, once about every month, once about every 2 months, once about every 3 months, once about every 4 months, once about every 5 months, once about every 6 months, or once about every 12 months following an event that would increase the patient's risk of acquiring HIV (e.g., following sexual intercourse or other exposure to the HIV). In certain embodiments, e.g., when administered as PEP, a composition provided herein is administered once about every week following an event that would increase the patient's risk of acquiring HIV (e.g., following sexual intercourse or other exposure to the HIV). In certain embodiments, e.g., when administered as PEP, a composition provided herein is administered once about every month following an event that would increase the patient's risk of acquiring HIV (e.g., following sexual intercourse or other exposure to the HIV). In certain embodiments, e.g., when administered as PEP, a composition provided herein is administered once about every 2 months following an event that would increase the patient's risk of acquiring HIV (e.g., following sexual intercourse or other exposure to the HIV). In certain embodiments, e.g., when administered as PEP, a composition provided herein is administered once about every 3 months following an event that would increase the patient's risk of acquiring HIV (e.g., following sexual intercourse or other exposure to the HIV). In certain embodiments, e.g., when administered as PEP, a composition provided herein is administered once about every 4 months following an event that would increase the patient's risk of acquiring HIV (e.g., following sexual intercourse or other exposure to the HIV). In certain embodiments, e.g., when administered as PEP, a composition provided herein is administered once about every 5 months following an event that would increase the patient's risk of acquiring HIV (e.g., following sexual intercourse or other exposure to the HIV). In certain embodiments, e.g., when administered as PEP, a composition provided herein is administered once about every 6 months following an event that would increase the patient's risk of acquiring HIV (e.g., following sexual intercourse or other exposure to the HIV). In certain embodiments, e.g., when administered as PEP, a composition provided herein is administered once about every 12 months following an event that would increase the patient's risk of acquiring HIV (e.g., following sexual intercourse or other exposure to the HIV).

In certain embodiments, e.g., when administered as PEP, a composition provided herein is administered for one month, two months, three months, four months, five months, six months, or twelve months following an event that would increase the patient's risk of acquiring HIV (e.g., following sexual intercourse or other exposure to the HIV).

In certain embodiments, when a composition provided herein is administered following an event that would increase the patient's risk of acquiring HIV, it is administered one to fifty times following the event. In certain embodiments, when a composition provided herein is administered following an event that would increase the patient's risk of acquiring HIV, it is administered one to forty times following the event. In certain embodiments, when a composition provided herein is administered following an event that would increase the patient's risk of acquiring HIV, it is administered one to thirty times following the event. In certain embodiments, when a composition provided herein is administered following an event that would increase the patient's risk of acquiring HIV, it is administered one to twenty times following the event. In certain embodiments, when a composition provided herein is administered following an event that would increase the patient's risk of acquiring HIV, it is administered one to fifteen times following the event. In certain embodiments, when a composition provided herein is administered following an event that would increase the patient's risk of acquiring HIV, it is administered one to ten times following the event. In certain embodiments, when a composition provided herein is administered following an event that would increase the patient's risk of acquiring HIV, it is administered one to five times following the event.

In some embodiments, a composition provided herein is administered during exposure of the patient to the HIV (e.g., during a period of sexual activity with a sexual partner known to be HIV positive).

In some embodiments, a composition provided herein is administered after exposure (e.g., after final exposure) of the patient to the HIV (e.g., after a period of sexual activity with a sexual partner known to be HIV positive). In some embodiments, a composition provided herein is administered from about 1 hour to about 14 days after exposure (e.g., after final exposure) of the patient to the HIV. In some embodiments, a composition provided herein is administered once from about 1 hour to about 14 days after exposure (e.g., after final exposure) of the patient to the HIV. In some embodiments, a composition provided herein is administered from about 1 hour to about 7 days after exposure (e.g., after final exposure) of the patient to the HIV. In some embodiments, a composition provided herein is administered once from about 1 hour to about 7 days after exposure (e.g., after final exposure) of the patient to the HIV. In some embodiments, a composition provided herein is administered from about 1 hour to about 72 hours after exposure (e.g., after final exposure) of the patient to the HIV. In some embodiments, a composition provided herein is administered once from about 1 hour to about 72 hours after exposure (e.g., after final exposure) of the patient to the HIV. In some embodiments, a composition provided herein is administered from about 1 hour to about 24 hours after exposure (e.g., after final exposure) of the patient to the HIV. In some embodiments, a composition provided herein is administered once from about 1 hour to about 24 hours after exposure (e.g., after final exposure) of the patient to the HIV. In some embodiments, a composition provided herein is administered from about 24 hours to about 72 hours after exposure (e.g., after final exposure) of the patient to the HIV. In some embodiments, a composition provided herein is administered once from about 24 hours to about 72 hours after exposure (e.g., after final exposure) of the patient to the HIV.

In some embodiments, e.g., when administered as PrEP, a composition provided herein is administered prior to an event that would increase the patient's risk of acquiring HIV (e.g., prior to sexual activity), and following the event. For example, in certain embodiments, when administered as PrEP, a composition provided herein is administered 1 to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 24 hours, or 1 hour to 12 hours prior to an event that would increase the patient's risk of acquiring HIV (e.g., prior to sexual activity) and 1 hour to 240 hours (i.e., within 10 days), 1 hour to 216 hours, 1 hour to 192 hours, 1 hour to 168 hours, 1 hour to 144 hours, 1 hour to 120 hours, 1 hour to 96 hours, 1 hour to 72 hours, 1 hour to 48 hours, 1 hour to 36 hours, 1 hour to 24 hours, or 1 hour to 12 hours following the event. For example, in some embodiments, one or more (e.g., one, two, or three) dosages of a composition provided herein are administered one to ten days (e.g., seven days) prior to an event that would increase the patient's risk of acquiring HIV (e.g., prior to sexual intercourse) and once during a period of one to ten days following the event. In some embodiments, a composition provided herein is administered once per week, twice per week, three times per week, four times per week, or five times per week and one or more times (e.g., one, two, or three times) beginning 1 to 48 hours following an event that would increase the patient's risk of acquiring HIV (e.g., following sexual intercourse).

Also provided herein is a method of reducing the risk of acquiring HIV in a patient, comprising administering to the patient a composition provided herein (e.g., a composition comprising a compound of Formula Ia or Ib, or a pharmaceutically acceptable salt or free acid form thereof).

In some embodiments, methods for reducing the risk of acquiring HIV (e.g., HIV-1 and/or HIV-2) comprise administration of a composition provided herein to a patient in combination with safer sexual intercourse practices. In certain embodiments, methods for reducing the risk of acquiring HIV (e.g., HIV-1 and/or HIV-2) comprise administration of a composition provided herein to a patient at risk of acquiring HIV. Examples of patients at high risk for acquiring HIV include, without limitation, a patient who is at risk of sexual transmission of HIV.

In some embodiments, the reduction in risk of acquiring HIV is at least about 40%, 50%, 60%, 70%, 80%, 90%, or 95% (compared to a patient having not been administered the compound of Formula Ia or Ib, or a pharmaceutically acceptable salt thereof according to any of the methods provided herein). In some embodiments, the reduction in risk of acquiring HIV is about 80%, 85%, or 90%. In some embodiments, the reduction in risk of acquiring HIV is at least about 75%. In some embodiments, the reduction in risk of acquiring HIV is at least about 80%. In some embodiments, the reduction in risk of acquiring HIV is at least about 85%. In some embodiments, the reduction in risk of acquiring HIV is at least about 90%.

In some embodiments, a composition provided herein (e.g., a tablet comprising a compound of Formula Ia or Ib, or a pharmaceutically acceptable salt or free acid form thereof) is suitable for oral administration. In some embodiments, a composition provided herein (e.g., a tablet comprising a compound of Formula Ia or Ib, or a pharmaceutically acceptable salt or free acid form thereof) is administered orally.

In some embodiments, a composition provided herein (e.g., a tablet comprising a compound of Formula Ia or Ib, or a pharmaceutically acceptable salt or free acid form thereof) is administered once every five to ten days, for example, once every five days, six days, seven days, eight days, nine days, or ten days. In some embodiments, a composition provided herein (e.g., a tablet comprising a compound of Formula Ia or Ib, or a pharmaceutically acceptable salt or free acid form thereof) is administered once every six to eight days. In some embodiments, a composition provided herein (e.g., a tablet comprising a compound of Formula Ia or Ib, or a pharmaceutically acceptable salt or free acid form thereof) is administered once every seven days (i.e., once-weekly).

In some embodiments, a composition provided herein (e.g., a tablet comprising a compound of Formula Ia or Ib, or a pharmaceutically acceptable salt or free acid form thereof) is orally administered once every five to ten days, for example, once every five days, six days, seven days, eight days, nine days, or ten days. In some embodiments, a composition provided herein (e.g., a tablet comprising a compound of Formula Ia or Ib, or a pharmaceutically acceptable salt or free acid form thereof) is orally administered once every six to eight days. In some embodiments, a composition provided herein (e.g., a tablet comprising a compound of Formula Ia or Ib, or a pharmaceutically acceptable salt or free acid form thereof) is orally administered once every seven days (i.e., once-weekly).

In some embodiments, a composition provided herein (e.g., a tablet comprising a compound of Formula Ia or Ib, or a pharmaceutically acceptable salt or free acid form thereof) is orally administered once every 25 to 35 days, for example, once every 25 days, once every 26 days, once every 27 days, once every 28 days, once every 29 days, once every 30 days, once every 31 days, once every 32 days, once every 33 days, once every 34 days, or once every 35 days.

In some embodiments, a composition provided herein (e.g., a tablet comprising a compound of Formula Ia or Ib, or a pharmaceutically acceptable salt or free acid form thereof) is orally administered once every 28 to 32 days. In some embodiments, a composition provided herein is orally administered once every 28 days. In some embodiments, a composition provided herein is orally administered once every 29 days. In some embodiments, a composition provided herein is orally administered once every 30 days. In some embodiments, a composition provided herein is orally administered once every 31 days. In some embodiments, a composition provided herein is orally administered once every 32 days. In some embodiments, a composition provided herein is orally administered once every month.

In some embodiments, about 1 mg to about 1500 mg of the compound of Formula Ia or Ib, or a pharmaceutically acceptable salt thereof, is administered to a patient in need thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg. about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, or about 1500 mg of the compound of Formula Ia or Ib, or a pharmaceutically acceptable salt thereof.

In some embodiments, about 1000 mg to about 1500 mg of the compound of Formula Ia or Ib, or a pharmaceutically acceptable salt thereof, is administered to a patient in need thereof, for example, about 1000 mg, about 1100 mg, about 1200 mg, about 1300 mg, about 1400 mg, or about 1500 mg of the compound of Formula Ia or Ib, or a pharmaceutically acceptable salt thereof. In some embodiments, about 1100 mg to about 1300 mg of the compound of Formula Ia or Ib, or a pharmaceutically acceptable salt thereof, is administered to a patient in need thereof. In some embodiments, about 1150 mg to about 1250 mg of the compound of Formula Ia or Ib, or a pharmaceutically acceptable salt thereof, is administered to a patient in need thereof. In some embodiments, about 1175 mg to about 1225 mg of the compound of Formula Ia or Ib, or a pharmaceutically acceptable salt thereof, is administered to a patient in need thereof. In some embodiments, about 1200 mg of the compound of Formula Ia or Ib, or a pharmaceutically acceptable salt thereof, is administered to a patient in need thereof.

In some embodiments, about 1 mg to about 500 mg of the compound of Formula Ia or Ib, or a pharmaceutically acceptable salt thereof, is administered to a patient in need thereof, for example, about 1 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

In some embodiments, the method comprises administering about 5 mg to about 500 mg of the compound of Formula Ia or Ib, or a pharmaceutically acceptable salt thereof to the patient. In some embodiments, the method comprises administering about 50 mg to about 450 mg of the compound of Formula Ia or Ib, or a pharmaceutically acceptable salt thereof to the patient. In some embodiments, the method comprises administering about 75 mg to about 425 mg of the compound of Formula Ia or Ib, or a pharmaceutically acceptable salt thereof to the patient.

In some embodiments, the method comprises administering about 75 mg to about 125 mg of the compound of Formula Ia or Ib, or a pharmaceutically acceptable salt thereof to the patient. In some embodiments, the method comprises administering about 100 mg of the compound of Formula Ia or Ib, or a pharmaceutically acceptable salt thereof to the patient.

In some embodiments, the method comprises administering about 175 mg to about 225 mg of the compound of Formula Ia or Ib, or a pharmaceutically acceptable salt thereof to the patient. In some embodiments, the method comprises administering about 200 mg of the compound of Formula Ia or Ib, or a pharmaceutically acceptable salt thereof to the patient.

In some embodiments, the method comprises administering about 275 mg to about 325 mg of the compound of Formula Ia or Ib, or a pharmaceutically acceptable salt thereof to the patient. In some embodiments, the method comprises administering about 300 mg of the compound of Formula Ia or Ib, or a pharmaceutically acceptable salt thereof to the patient.

In some embodiments, the method comprises administering about 375 mg to about 425 mg of the compound of Formula Ia or Ib, or a pharmaceutically acceptable salt thereof to the patient. In some embodiments, the method comprises administering about 400 mg of the compound of Formula Ia or Ib, or a pharmaceutically acceptable salt thereof to the patient.

In some embodiments, the method comprises orally administering about 5 mg to about 500 mg of the compound of Formula Ia or Ib, or a pharmaceutically acceptable salt thereof to the patient. In some embodiments, the method comprises orally administering about 50 mg to about 450 mg of the compound of Formula Ia or Ib, or a pharmaceutically acceptable salt thereof to the patient. In some embodiments, the method comprises orally administering about 75 mg to about 425 mg of the compound of Formula Ia or Ib, or a pharmaceutically acceptable salt thereof to the patient.

In some embodiments, the method comprises orally administering about 75 mg to about 125 mg of the compound of Formula Ia or Ib, or a pharmaceutically acceptable salt thereof to the patient. In some embodiments, the method comprises orally administering about 100 mg of the compound of Formula Ia or Ib, or a pharmaceutically acceptable salt thereof to the patient.

In some embodiments, the method comprises orally administering about 175 mg to about 225 mg of the compound of Formula Ia or Ib, or a pharmaceutically acceptable salt thereof to the patient. In some embodiments, the method comprises orally administering about 200 mg of the compound of Formula Ia or Ib, or a pharmaceutically acceptable salt thereof to the patient.

In some embodiments, the method comprises orally administering about 275 mg to about 325 mg of the compound of Formula Ia or Ib, or a pharmaceutically acceptable salt thereof to the patient. In some embodiments, the method comprises orally administering about 300 mg of the compound of Formula Ia or Ib, or a pharmaceutically acceptable salt thereof to the patient.

In some embodiments, the method comprises orally administering about 375 mg to about 425 mg of the compound of Formula Ia or Ib, or a pharmaceutically acceptable salt thereof to the patient. In some embodiments, the method comprises orally administering about 400 mg of the compound of Formula Ia or Ib, or a pharmaceutically acceptable salt thereof to the patient.

In some embodiments, the method comprises orally administering about 5 mg to about 500 mg of the compound of Formula Ia or Ib, or a pharmaceutically acceptable salt thereof to the patient, once every seven days. In some embodiments, the method comprises orally administering about 50 mg to about 450 mg of the compound of Formula Ia or Ib, or a pharmaceutically acceptable salt thereof to the patient, once every seven days. In some embodiments, the method comprises orally administering about 75 mg to about 425 mg of the compound of Formula Ia or Ib, or a pharmaceutically acceptable salt thereof to the patient, once every seven days.

In some embodiments, the method comprises orally administering about 75 mg to about 125 mg of the compound of Formula Ia or Ib, or a pharmaceutically acceptable salt thereof to the patient, once every seven days. In some embodiments, the method comprises orally administering about 100 mg of the compound of Formula Ia or Ib, or a pharmaceutically acceptable salt thereof to the patient, once every seven days.

In some embodiments, the method comprises orally administering about 175 mg to about 225 mg of the compound of Formula Ia or Ib, or a pharmaceutically acceptable salt thereof to the patient, once every seven days. In some embodiments, the method comprises orally administering about 200 mg of the compound of Formula Ia or Ib, or a pharmaceutically acceptable salt thereof to the patient, once every seven days.

In some embodiments, the method comprises orally administering about 275 mg to about 325 mg of the compound of Formula Ia or Ib, or a pharmaceutically acceptable salt thereof to the patient, once every seven days. In some embodiments, the method comprises orally administering about 300 mg of the compound of Formula Ia or Ib, or a pharmaceutically acceptable salt thereof to the patient, once every seven days.

In some embodiments, the method comprises orally administering about 375 mg to about 425 mg of the compound of Formula Ia or Ib, or a pharmaceutically acceptable salt thereof to the patient, once every seven days. In some embodiments, the method comprises orally administering about 400 mg of the compound of Formula Ia or Ib, or a pharmaceutically acceptable salt thereof to the patient, once every seven days.

In some embodiments, the method comprises administering (e.g., orally administering, such as orally administering once every seven days) to a patient a tablet comprising:
about 5 mg to about 500 mg of the compound of Formula Ia or Ib, or a pharmaceutically acceptable salt thereof;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the method comprises administering (e.g., orally administering, such as orally administering once every seven days) to a patient a tablet comprising:
about 75 mg to about 125 mg of the compound of Formula Ia or Ib, or a pharmaceutically acceptable salt thereof;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the method comprises administering (e.g., orally administering, such as orally administering once every seven days) to a patient a tablet comprising:
about 100 mg of the compound of Formula Ia or Ib, or a pharmaceutically acceptable salt thereof;
about 67.5 mg of mannitol;
about 68.75 mg of microcrystalline cellulose;
about 10 mg of crospovidone; and
about 3.75 mg of magnesium stearate.

In some embodiments, the method comprises administering (e.g., orally administering, such as orally administering once every seven days) to a patient a tablet comprising:
about 75 mg to about 125 mg of the compound of Formula Ia, or a pharmaceutically acceptable salt thereof;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the method comprises administering (e.g., orally administering, such as orally administering once every seven days) to a patient a tablet comprising:
about 100 mg of the compound of Formula Ia, or a pharmaceutically acceptable salt thereof;
about 67.5 mg of mannitol;
about 68.75 mg of microcrystalline cellulose;
about 10 mg of crospovidone; and
about 3.75 mg of magnesium stearate.

In some embodiments, the method comprises administering (e.g., orally administering, such as orally administering once every seven days) to a patient a tablet comprising:
about 75 mg to about 125 mg of the compound of Formula Ib, or a pharmaceutically acceptable salt thereof;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the method comprises administering (e.g., orally administering, such as orally administering once every seven days) to a patient a tablet comprising:
about 100 mg of the compound of Formula Ib, or a pharmaceutically acceptable salt thereof;
about 67.5 mg of mannitol;
about 68.75 mg of microcrystalline cellulose;
about 10 mg of crospovidone; and
about 3.75 mg of magnesium stearate.

In some embodiments, the method comprises administering (e.g., orally administering, such as orally administering once every seven days) to a patient a tablet comprising:
about 75 mg to about 125 mg of a crystalline form of the compound of Formula Ib;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the method comprises administering (e.g., orally administering, such as orally administering once every seven days) to a patient a tablet comprising:
about 100 mg of a crystalline form of the compound of Formula Ib;
about 67.5 mg of mannitol;
about 68.75 mg of microcrystalline cellulose;
about 10 mg of crospovidone; and
about 3.75 mg of magnesium stearate.

In some embodiments, the method comprises administering (e.g., orally administering, such as orally administering once every seven days) to a patient a tablet comprising:
about 75 mg to about 125 mg of the compound of Formula Ib, crystalline Form I;
about 50 mg to about 100 mg of mannitol;
about 50 mg to about 100 mg of microcrystalline cellulose;
about 5 mg to about 15 mg of crospovidone; and
about 1 mg to about 6 mg of magnesium stearate.

In some embodiments, the method comprises administering (e.g., orally administering, such as orally administering once every seven days) to a patient a tablet comprising:
about 100 mg of the compound of Formula Ib, crystalline Form I;
about 67.5 mg of mannitol;
about 68.75 mg of microcrystalline cellulose;
about 10 mg of crospovidone; and
about 3.75 mg of magnesium stearate.

In some embodiments, the method comprises administering (e.g., orally administering, such as orally administering once every seven days) to a patient a tablet comprising:
about 175 mg to about 225 mg of the compound of Formula Ia or Ib, or a pharmaceutically acceptable salt thereof;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of the compound of Formula Ia or Ib, or a pharmaceutically acceptable salt thereof;
about 135 mg of mannitol;
about 137.5 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 175 mg to about 225 mg of the compound of Formula Ia or Ib, or a pharmaceutically acceptable salt thereof;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of the compound of Formula Ia or Ib, or a pharmaceutically acceptable salt thereof;
about 135 mg to about 137 mg of mannitol;
about 135 mg to about 137 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ia or Ib, or a pharmaceutically acceptable salt thereof;
about 136.2 mg of mannitol;
about 136.2 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ia or Ib, or a pharmaceutically acceptable salt thereof;
about 136.3 mg of mannitol;
about 136.3 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ia or Ib, or a pharmaceutically acceptable salt thereof;
about 136.25 mg of mannitol;
about 136.25 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 5 mg to about 500 mg of the compound of Formula Ia, or a pharmaceutically acceptable salt thereof,
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the method comprises administering (e.g., orally administering, such as orally administering once every seven days) to a patient a tablet comprising:
about 175 mg to about 225 mg of the compound of Formula Ia, or a pharmaceutically acceptable salt thereof;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of the compound of Formula Ia, or a pharmaceutically acceptable salt thereof;
about 135 mg of mannitol;
about 137.5 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 175 mg to about 225 mg of the compound of Formula Ia, or a pharmaceutically acceptable salt thereof;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of the compound of Formula Ia, or a pharmaceutically acceptable salt thereof;
about 135 mg to about 137 mg of mannitol;
about 135 mg to about 137 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ia, or a pharmaceutically acceptable salt thereof;
about 136.2 mg of mannitol;
about 136.2 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ia, or a pharmaceutically acceptable salt thereof;
about 136.3 mg of mannitol;
about 136.3 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ia, or a pharmaceutically acceptable salt thereof;
about 136.25 mg of mannitol;
about 136.25 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 5 mg to about 500 mg of the compound of Formula Ia;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the method comprises administering (e.g., orally administering, such as orally administering once every seven days) to a patient a tablet comprising:
about 175 mg to about 225 mg of the compound of Formula Ia;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of the compound of Formula Ia;
about 135 mg of mannitol;
about 137.5 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 175 mg to about 225 mg of the compound of Formula Ia;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of the compound of Formula Ia;
about 135 mg to about 137 mg of mannitol;
about 135 mg to about 137 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ia;
about 136.2 mg of mannitol;
about 136.2 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ia;
about 136.3 mg of mannitol;
about 136.3 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ia;
about 136.25 mg of mannitol;
about 136.25 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 5 mg to about 500 mg of the compound of Formula Ib, or a pharmaceutically acceptable salt thereof;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the method comprises administering (e.g., orally administering, such as orally administering once every seven days) to a patient a tablet comprising:
about 175 mg to about 225 mg of the compound of Formula Ib, or a pharmaceutically acceptable salt thereof;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of the compound of Formula Ib, or a pharmaceutically acceptable salt thereof;
about 135 mg of mannitol;
about 137.5 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 175 mg to about 225 mg of the compound of Formula Ib, or a pharmaceutically acceptable salt thereof,
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of the compound of Formula Ib, or a pharmaceutically acceptable salt thereof;
about 135 mg to about 137 mg of mannitol;
about 135 mg to about 137 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ib, or a pharmaceutically acceptable salt thereof;
about 136.2 mg of mannitol;
about 136.2 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ib, or a pharmaceutically acceptable salt thereof;
about 136.3 mg of mannitol;
about 136.3 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ib, or a pharmaceutically acceptable salt thereof;
about 136.25 mg of mannitol;
about 136.25 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 5 mg to about 500 mg of the compound of Formula Ib;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the method comprises administering (e.g., orally administering, such as orally administering once every seven days) to a patient a tablet comprising:
about 175 mg to about 225 mg of the compound of Formula Ib;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of the compound of Formula Ib;
about 135 mg of mannitol;
about 137.5 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 175 mg to about 225 mg of the compound of Formula Ib;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of the compound of Formula Ib;
about 135 mg to about 137 mg of mannitol;
about 135 mg to about 137 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ib;
about 136.2 mg of mannitol;
about 136.2 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ib;
about 136.3 mg of mannitol;
about 136.3 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of the compound of Formula Ib;
about 136.25 mg of mannitol;
about 136.25 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 5 mg to about 500 mg of a crystalline form of the compound of Formula Ib;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the method comprises administering (e.g., orally administering, such as orally administering once every seven days) to a patient a tablet comprising:
about 175 mg to about 225 mg of a crystalline form of the compound of Formula Ib;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of a crystalline form of the compound of Formula Ib;
about 135 mg of mannitol;
about 137.5 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 175 mg to about 225 mg of a crystalline form of the compound of Formula Ib;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of a crystalline form of the compound of Formula Ib;
about 135 mg to about 137 mg of mannitol;
about 135 mg to about 137 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of a crystalline form of the compound of Formula Ib;
about 136.2 mg of mannitol;
about 136.2 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of a crystalline form of the compound of Formula Ib;
about 136.3 mg of mannitol;
about 136.3 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of a crystalline form of the compound of Formula Ib;
about 136.25 mg of mannitol;
about 136.25 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 5 mg to about 500 mg of a compound of Formula Ib, crystalline Form I;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

In some embodiments, the method comprises administering (e.g., orally administering, such as orally administering once every seven days) to a patient a tablet comprising:
about 175 mg to about 225 mg of a compound of Formula Ib, crystalline Form I;
about 100 mg to about 150 mg of mannitol;
about 100 mg to about 150 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of a compound of Formula Ib, crystalline Form I;
about 135 mg of mannitol;
about 137.5 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 175 mg to about 225 mg of a compound of Formula Ib, crystalline Form I;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the method comprises administering (e.g., orally administering, such as orally administering once every seven days) to a patient a tablet comprising:
about 200 mg of a compound of Formula Ib, crystalline Form I;
about 135 mg to about 137 mg of mannitol;
about 135 mg to about 137 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of a compound of Formula Ib, crystalline Form I;
about 136.2 mg of mannitol;
about 136.2 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of a compound of Formula Ib, crystalline Form I;
about 136.3 mg of mannitol;
about 136.3 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 200 mg of a compound of Formula Ib, crystalline Form I;
about 136.25 mg of mannitol;
about 136.25 mg of microcrystalline cellulose;
about 20 mg of crospovidone; and
about 7.5 mg of magnesium stearate.

In some embodiments, the method comprises administering (e.g., orally administering, such as orally administering once every seven days) to a patient a tablet comprising:
about 275 mg to about 325 mg of the compound of Formula Ia or Ib, or a pharmaceutically acceptable salt thereof;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the method comprises administering (e.g., orally administering, such as orally administering once every seven days) to a patient a tablet comprising:
about 300 mg of the compound of Formula Ia or Ib, or a pharmaceutically acceptable salt thereof;
about 202.5 mg of mannitol;
about 206.25 mg of microcrystalline cellulose;
about 30 mg of crospovidone; and
about 11.25 mg of magnesium stearate.

In some embodiments, the method comprises administering (e.g., orally administering, such as orally administering once every seven days) to a patient a tablet comprising:
about 275 mg to about 325 mg of the compound of Formula Ia, or a pharmaceutically acceptable salt thereof,
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the method comprises administering (e.g., orally administering, such as orally administering once every seven days) to a patient a tablet comprising:
about 300 mg of the compound of Formula Ia, or a pharmaceutically acceptable salt thereof;
about 202.5 mg of mannitol;
about 206.25 mg of microcrystalline cellulose;
about 30 mg of crospovidone; and
about 11.25 mg of magnesium stearate.

In some embodiments, the method comprises administering (e.g., orally administering, such as orally administering once every seven days) to a patient a tablet comprising:
about 275 mg to about 325 mg of the compound of Formula Ib, or a pharmaceutically acceptable salt thereof;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the method comprises administering (e.g., orally administering, such as orally administering once every seven days) to a patient a tablet comprising:
about 300 mg of the compound of Formula Ib, or a pharmaceutically acceptable salt thereof;
about 202.5 mg of mannitol;
about 206.25 mg of microcrystalline cellulose;
about 30 mg of crospovidone; and
about 11.25 mg of magnesium stearate.

In some embodiments, the method comprises administering (e.g., orally administering, such as orally administering once every seven days) to a patient a tablet comprising:
about 275 mg to about 325 mg of a crystalline form of the compound of Formula Ib;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the method comprises administering (e.g., orally administering, such as orally administering once every seven days) to a patient a tablet comprising:
about 300 mg of a crystalline form of the compound of Formula Ib;
about 202.5 mg of mannitol;
about 206.25 mg of microcrystalline cellulose;
about 30 mg of crospovidone; and
about 11.25 mg of magnesium stearate.

In some embodiments, the method comprises administering (e.g., orally administering, such as orally administering once every seven days) to a patient a tablet comprising:
about 275 mg to about 325 mg of a compound of Formula Ib, crystalline Form I;
about 150 mg to about 250 mg of mannitol;
about 150 mg to about 250 mg of microcrystalline cellulose;
about 25 mg to about 35 mg of crospovidone; and
about 9 mg to about 14 mg of magnesium stearate.

In some embodiments, the method comprises administering (e.g., orally administering, such as orally administering once every seven days) to a patient a tablet comprising:
about 300 mg of a compound of Formula Ib, crystalline Form I;
about 202.5 mg of mannitol;
about 206.25 mg of microcrystalline cellulose;
about 30 mg of crospovidone; and
about 11.25 mg of magnesium stearate.

In some embodiments, the method comprises administering (e.g., orally administering, such as orally administering once every seven days) to a patient a tablet comprising:
about 375 mg to about 425 mg of the compound of Formula Ia or Tb, or a pharmaceutically acceptable salt thereof;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the method comprises administering (e.g., orally administering, such as orally administering once every seven days) to a patient a tablet comprising:
about 400 mg of the compound of Formula Ia or Ib, or a pharmaceutically acceptable salt thereof;
about 270 mg of mannitol;
about 275 mg of microcrystalline cellulose;
about 40 mg of crospovidone; and
about 15 mg of magnesium stearate.

In some embodiments, the method comprises administering (e.g., orally administering, such as orally administering once every seven days) to a patient a tablet comprising:
about 375 mg to about 425 mg of the compound of Formula Ia, or a pharmaceutically acceptable salt thereof;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the method comprises administering (e.g., orally administering, such as orally administering once every seven days) to a patient a tablet comprising:
about 400 mg of the compound of Formula Ia, or a pharmaceutically acceptable salt thereof;
about 270 mg of mannitol;
about 275 mg of microcrystalline cellulose;
about 40 mg of crospovidone; and
about 15 mg of magnesium stearate.

In some embodiments, the method comprises administering (e.g., orally administering, such as orally administering once every seven days) to a patient a tablet comprising:
about 375 mg to about 425 mg of the compound of Formula Ib, or a pharmaceutically acceptable salt thereof,
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the method comprises administering (e.g., orally administering, such as orally administering once every seven days) to a patient a tablet comprising:
about 400 mg of the compound of Formula Ib, or a pharmaceutically acceptable salt thereof;
about 270 mg of mannitol;
about 275 mg of microcrystalline cellulose;
about 40 mg of crospovidone; and
about 15 mg of magnesium stearate.

In some embodiments, the method comprises administering (e.g., orally administering, such as orally administering once every seven days) to a patient a tablet comprising:
about 375 mg to about 425 mg of a crystalline form of a compound of Formula Ib;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the method comprises administering (e.g., orally administering, such as orally administering once every seven days) to a patient a tablet comprising:
about 400 mg of a crystalline form of a compound of Formula Ib;
about 270 mg of mannitol;
about 275 mg of microcrystalline cellulose;
about 40 mg of crospovidone; and
about 15 mg of magnesium stearate.

In some embodiments, the method comprises administering (e.g., orally administering, such as orally administering once every seven days) to a patient a tablet comprising:
about 375 mg to about 425 mg of a compound of Formula Ib, crystalline Form I;
about 250 mg to about 300 mg of mannitol;
about 250 mg to about 300 mg of microcrystalline cellulose;
about 35 mg to about 45 mg of crospovidone; and
about 10 mg to about 20 mg of magnesium stearate.

In some embodiments, the method comprises administering (e.g., orally administering, such as orally administering once every seven days) to a patient a tablet comprising:
about 400 mg of a compound of Formula Ib, crystalline Form I;
about 270 mg of mannitol;
about 275 mg of microcrystalline cellulose;
about 40 mg of crospovidone; and
about 15 mg of magnesium stearate.

In some embodiments, the method comprises administering (e.g., orally administering, such as orally administering once every month) to a patient a tablet comprising:
about 1000 mg to about 1500 mg of the compound of Formula Ia or Ib, or a pharmaceutically acceptable salt thereof,
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the method comprises administering (e.g., orally administering, such as orally administering once every month) to a patient a tablet comprising:
about 1100 mg to about 1300 mg of the compound of Formula Ia or Ib, or a pharmaceutically acceptable salt thereof;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the method comprises administering (e.g., orally administering, such as orally administering once every month) to a patient a tablet comprising:
about 1200 mg of the compound of Formula Ia or Ib, or a pharmaceutically acceptable salt thereof;
about 124 mg of mannitol;
about 124 mg of microcrystalline cellulose;
about 128 mg of crospovidone;
about 24 mg of magnesium stearate.

In some embodiments, the method comprises administering (e.g., orally administering, such as orally administering once every month) to a patient a tablet comprising:

about 1000 mg to about 1500 mg of the compound of Formula Ia, or a pharmaceutically acceptable salt thereof,
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the method comprises administering (e.g., orally administering, such as orally administering once every month) to a patient a tablet comprising:
about 1100 mg to about 1300 mg of the compound of Formula Ia, or a pharmaceutically acceptable salt thereof;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the method comprises administering (e.g., orally administering, such as orally administering once every month) to a patient a tablet comprising:
about 1200 mg of the compound of Formula Ia, or a pharmaceutically acceptable salt thereof;
about 124 mg of mannitol;
about 124 mg of microcrystalline cellulose;
about 128 mg of crospovidone;
about 24 mg of magnesium stearate.

In some embodiments, the method comprises administering (e.g., orally administering, such as orally administering once every month) to a patient a tablet comprising:
about 1000 mg to about 1500 mg of the compound of Formula Ia;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the method comprises administering (e.g., orally administering, such as orally administering once every month) to a patient a tablet comprising:
about 1100 mg to about 1300 mg of the compound of Formula Ia;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the method comprises administering (e.g., orally administering, such as orally administering once every month) to a patient a tablet comprising:
about 1200 mg of the compound of Formula Ia;
about 124 mg of mannitol;
about 124 mg of microcrystalline cellulose;
about 128 mg of crospovidone;
about 24 mg of magnesium stearate.

In some embodiments, the method comprises administering (e.g., orally administering, such as orally administering once every month) to a patient a tablet comprising:
about 1000 mg to about 1500 mg of the compound of Formula Ib, or a pharmaceutically acceptable salt thereof;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the method comprises administering (e.g., orally administering, such as orally administering once every month) to a patient a tablet comprising:
about 1100 mg to about 1300 mg of the compound of Formula Ib, or a pharmaceutically acceptable salt thereof;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the method comprises administering (e.g., orally administering, such as orally administering once every month) to a patient a tablet comprising:
about 1200 mg of the compound of Formula Ib, or a pharmaceutically acceptable salt thereof;
about 124 mg of mannitol;
about 124 mg of microcrystalline cellulose;
about 128 mg of crospovidone;
about 24 mg of magnesium stearate.

In some embodiments, the method comprises administering (e.g., orally administering, such as orally administering once every month) to a patient a tablet comprising:
about 1000 mg to about 1500 mg of the compound of Formula Ib;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the method comprises administering (e.g., orally administering, such as orally administering once every month) to a patient a tablet comprising:
about 1100 mg to about 1300 mg of the compound of Formula Ib;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the method comprises administering (e.g., orally administering, such as orally administering once every month) to a patient a tablet comprising:
about 1200 mg of the compound of Formula Ib;
about 124 mg of mannitol;
about 124 mg of microcrystalline cellulose;
about 128 mg of crospovidone;
about 24 mg of magnesium stearate.

In some embodiments, the method comprises administering (e.g., orally administering, such as orally administering once every month) to a patient a tablet comprising:
about 1000 mg to about 1500 mg of a crystalline form of the compound of Formula Ib;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the method comprises administering (e.g., orally administering, such as orally administering once every month) to a patient a tablet comprising:
about 1100 mg to about 1300 mg of a crystalline form of the compound of Formula Ib;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the method comprises administering (e.g., orally administering, such as orally administering once every month) to a patient a tablet comprising:
about 1200 mg of a crystalline form of the compound of Formula Ib;
about 124 mg of mannitol;
about 124 mg of microcrystalline cellulose;
about 128 mg of crospovidone;
about 24 mg of magnesium stearate.

In some embodiments, the method comprises administering (e.g., orally administering, such as orally administering once every month) to a patient a tablet comprising:
about 1000 mg to about 1500 mg of a compound of Formula Ib, crystalline Form I;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the method comprises administering (e.g., orally administering, such as orally administering once every month) to a patient a tablet comprising:
about 1100 mg to about 1300 mg of a compound of Formula Ib, crystalline Form I;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the method comprises administering (e.g., orally administering, such as orally administering once every month) to a patient a tablet comprising:
about 1200 mg of a compound of Formula Ib, crystalline Form I;
about 124 mg of mannitol;
about 124 mg of microcrystalline cellulose;
about 128 mg of crospovidone;
about 24 mg of magnesium stearate.

In some embodiments, the compositions provided herein are administered as a monotherapy.

In some embodiments, the present disclosure relates to a method of reducing the risk of sexually acquired HIV in a patient, comprising administering to the patient a compound of Formula Ia:

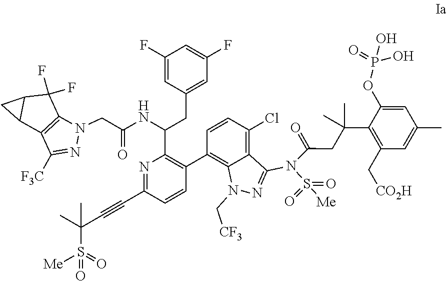 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, the method comprising orally administering to the patient about 175 mg to about 225 mg of the compound of Formula Ia, or a pharmaceutically acceptable salt thereof, once every seven days.

In some embodiments, the present disclosure relates to a method of reducing the risk of sexually acquired HIV in a patient, comprising administering to the patient a compound of Formula Ia:

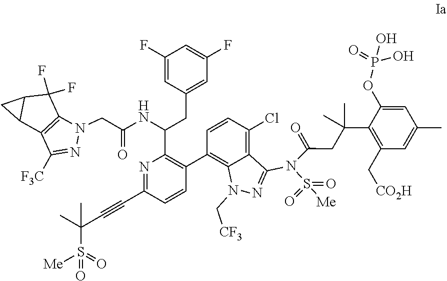 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

the method comprising orally administering to the patient about 175 mg to about 225 mg of the compound of Formula Ia, once every seven days.

In some embodiments, the present disclosure relates to a method of reducing the risk of sexually acquired HIV in a patient, comprising administering to the patient a compound of Formula Ib:

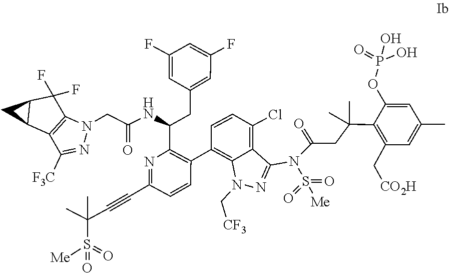 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, the method comprising orally administering to the patient about 175 mg to about 225 mg of the compound of Formula Ib, or a pharmaceutically acceptable salt thereof, once every seven days.

In some embodiments, the present disclosure relates to a method of reducing the risk of sexually acquired HIV in a patient, comprising administering to the patient a compound of Formula Ib:

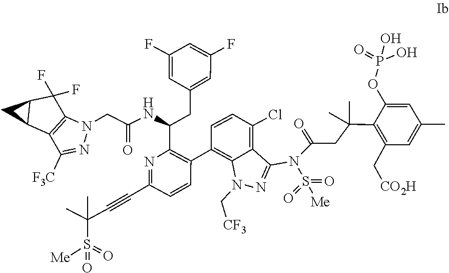 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 175 mg to about 225 mg of the compound of Formula Ib, once every seven days.

In some embodiments, the present disclosure relates to a method of reducing the risk of sexually acquired HIV in a patient, comprising administering to the patient a crystalline form of the compound of Formula Ib:

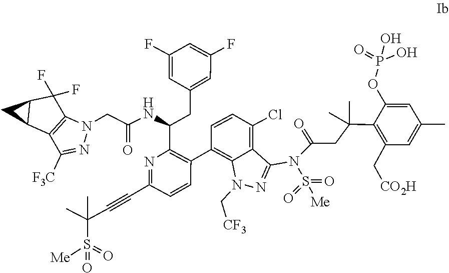 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 175 mg to about 225 mg of the crystalline form of the compound of Formula Ib, once every seven days.

In some embodiments, the present disclosure relates to a method of reducing the risk of sexually acquired HIV in a patient, comprising administering to the patient a compound of Formula Ib, crystalline Form I:

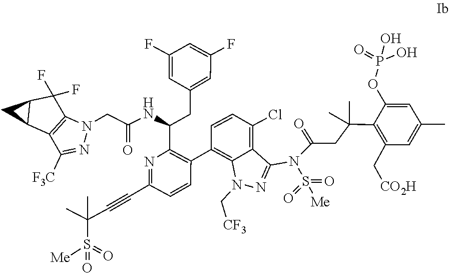 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 175 mg to about 225 mg of the compound of Formula Ib, crystalline Form I, once every seven days.

In some embodiments, the present disclosure relates to a method of reducing the risk of sexually acquired HIV in a patient, comprising administering to the patient a compound of Formula Ia:

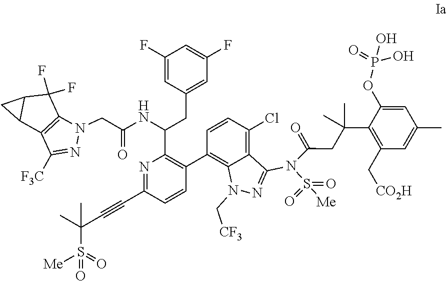 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, the method comprising orally administering to the patient about 200 mg of the compound of Formula Ia, or a pharmaceutically acceptable salt thereof, once every seven days.

In some embodiments, the present disclosure relates to a method of reducing the risk of sexually acquired HIV in a patient, comprising administering to the patient a compound of Formula Ia:

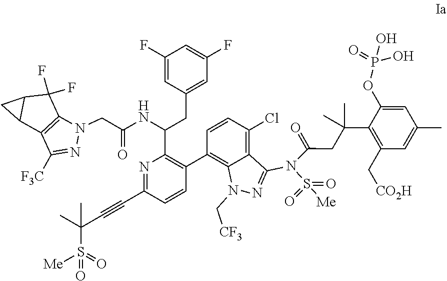 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

the method comprising orally administering to the patient about 200 mg of the compound of Formula Ia, once every seven days.

In some embodiments, the present disclosure relates to a method of reducing the risk of sexually acquired HIV in a patient, comprising administering to the patient a compound of Formula Ib:

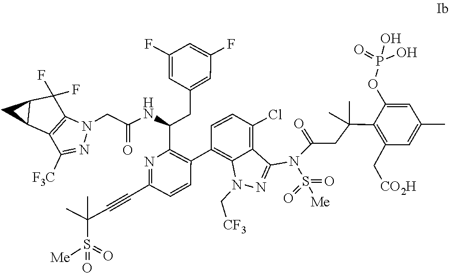 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, the method comprising orally administering to the patient about 200 mg of the compound of Formula Ib, or a pharmaceutically acceptable salt thereof, once every seven days.

In some embodiments, the present disclosure relates to a method of reducing the risk of sexually acquired HIV in a patient, comprising administering to the patient a compound of Formula Ib:

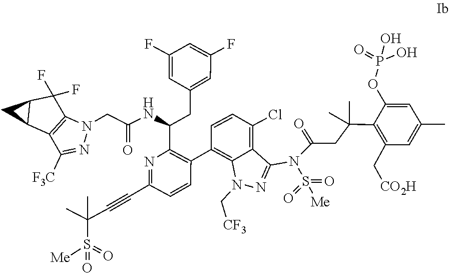 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 200 mg of the compound of Formula Ib, once every seven days.

In some embodiments, the present disclosure relates to a method of reducing the risk of sexually acquired HIV in a patient, comprising administering to the patient a crystalline form of the compound of Formula Ib:

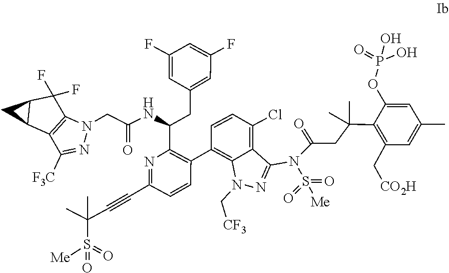 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 200 mg of the crystalline form of the compound of Formula Ib, once every seven days.

In some embodiments, the present disclosure relates to a method of reducing the risk of sexually acquired HIV in a patient, comprising administering to the patient a compound of Formula Ib, crystalline Form I:

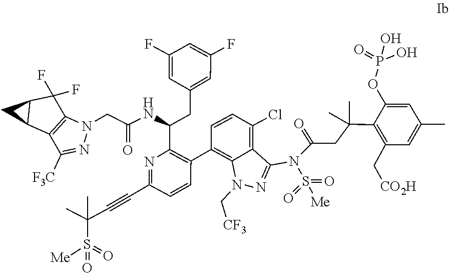 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 200 mg of the compound of Formula Ib, crystalline Form I, once every seven days.

In some embodiments, the present disclosure relates to a method of reducing the risk of sexually acquired HIV in a patient, comprising administering to the patient a compound of Formula Ia:

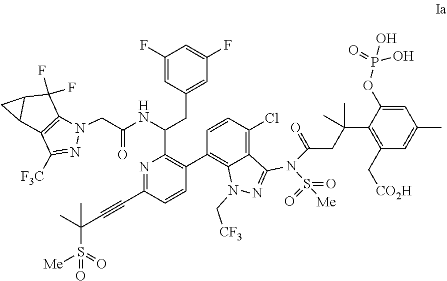 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, the method comprising orally administering to the patient about 1000 mg to about 1500 mg of the compound of Formula Ia, or a pharmaceutically acceptable salt thereof, once every month.

In some embodiments, the present disclosure relates to a method of reducing the risk of sexually acquired HIV in a patient, comprising administering to the patient a compound of Formula Ia:

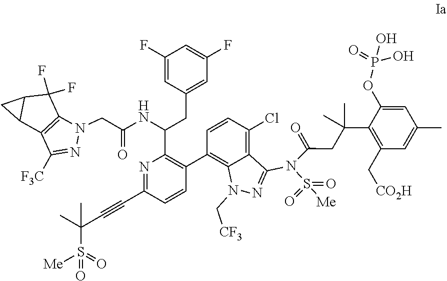 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

the method comprising orally administering to the patient about 1000 mg to about 1500 mg of the compound of Formula Ia, once every month.

In some embodiments, the present disclosure relates to a method of reducing the risk of sexually acquired HIV in a patient, comprising administering to the patient a compound of Formula Ib:

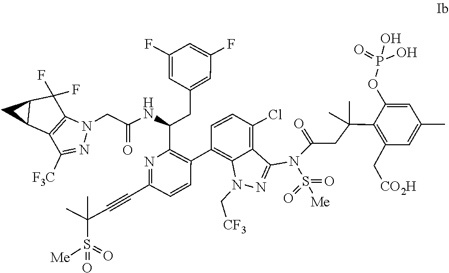 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, the method comprising orally administering to the patient about 1000 mg to about 1500 mg of the compound of Formula Ib, or a pharmaceutically acceptable salt thereof, once every month.

In some embodiments, the present disclosure relates to a method of reducing the risk of sexually acquired HIV in a patient, comprising administering to the patient a compound of Formula Ib:

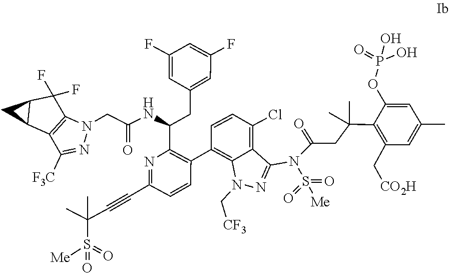 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 1000 mg to about 1500 mg of the compound of Formula Ib, once every month.

In some embodiments, the present disclosure relates to a method of reducing the risk of sexually acquired HIV in a patient, comprising administering to the patient a crystalline form of the compound of Formula Ib:

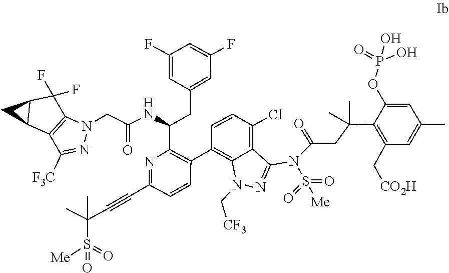 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 1000 mg to about 1500 mg of the crystalline form of the compound of Formula Ib, once every month.

In some embodiments, the present disclosure relates to a method of reducing the risk of sexually acquired HIV in a patient, comprising administering to the patient a compound of Formula Ib, crystalline Form I:

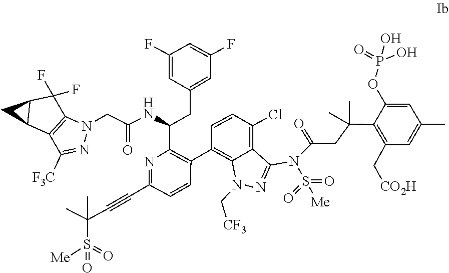 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 1000 mg to about 1500 mg of the compound of Formula Ib, crystalline Form I, once every month.

In some embodiments, the present disclosure relates to a method of reducing the risk of sexually acquired HIV in a patient, comprising administering to the patient a compound of Formula Ia:

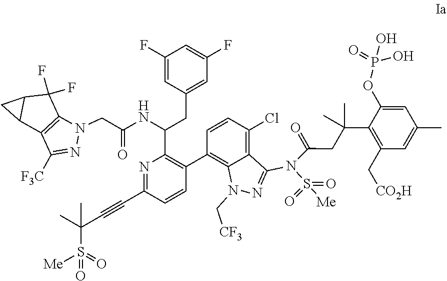 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, the method comprising orally administering to the patient about 1200 mg of the compound of Formula Ia, or a pharmaceutically acceptable salt thereof, once every month.

In some embodiments, the present disclosure relates to a method of reducing the risk of sexually acquired HIV in a patient, comprising administering to the patient a compound of Formula Ia:

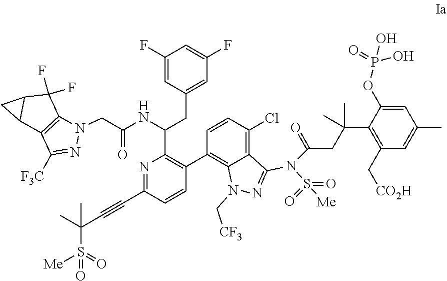 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

the method comprising orally administering to the patient about 1200 mg of the compound of Formula Ia, once every month.

In some embodiments, the present disclosure relates to a method of reducing the risk of sexually acquired HIV in a patient, comprising administering to the patient a compound of Formula Ib:

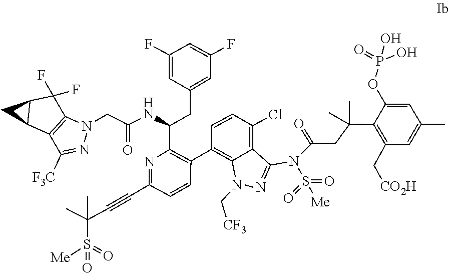 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, the method comprising orally administering to the patient about 1200 mg of the compound of Formula Ib, or a pharmaceutically acceptable salt thereof, once every month.

In some embodiments, the present disclosure relates to a method of reducing the risk of sexually acquired HIV in a patient, comprising administering to the patient a compound of Formula Ib:

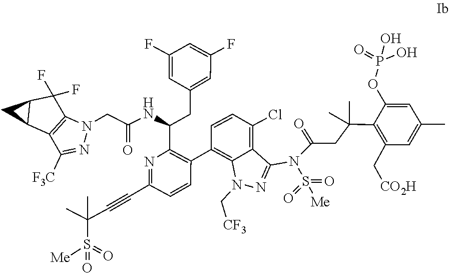 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 1200 mg of the compound of Formula Ib, once every month.

In some embodiments, the present disclosure relates to a method of reducing the risk of sexually acquired HIV in a patient, comprising administering to the patient a crystalline form of the compound of Formula Ib:

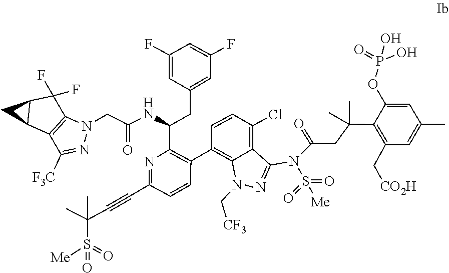 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 1200 mg of the crystalline form of the compound of Formula Ib, once every month.

In some embodiments, the present disclosure relates to a method of reducing the risk of sexually acquired HIV in a patient, comprising administering to the patient a compound of Formula Ib, crystalline Form I:

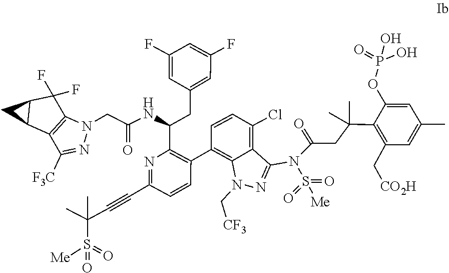 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

the method comprising orally administering to the patient about 1200 mg of the compound of Formula Ib, crystalline Form I, once every month.

In some embodiments, the present disclosure relates to a method of reducing the risk of sexually acquired HIV in a patient, comprising orally administering to the patient a tablet comprising a compound of Formula Ia:

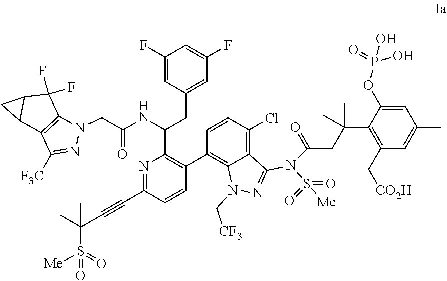 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every seven days, wherein the tablet comprises:
about 35 w/w % to about 45 w/w % of the compound of Formula Ia, or a pharmaceutically acceptable salt thereof;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ia:

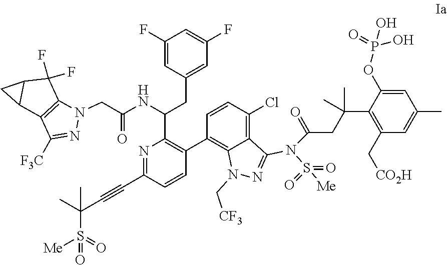 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every seven days, wherein the tablet comprises:
about 35 w/w % to about 45 w/w % of the compound of Formula Ia, or a pharmaceutically acceptable salt thereof;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ia:

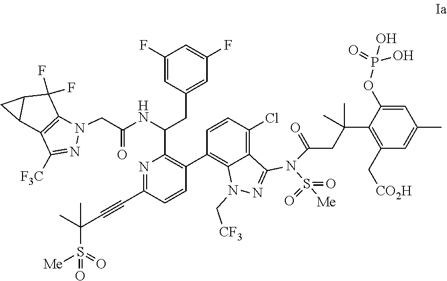 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every seven days, wherein the tablet comprises:
about 35 w/w % to about 45 w/w % of the compound of Formula Ia, or a pharmaceutically acceptable salt thereof;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ia:

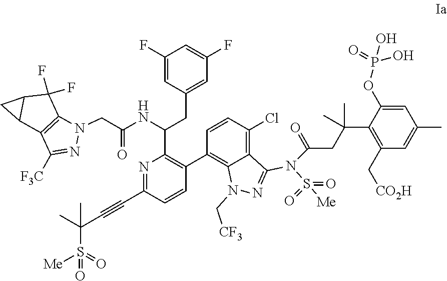 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every seven days, wherein the tablet comprises:
about 35 w/w % to about 45 w/w % of the compound of Formula Ia, or a pharmaceutically acceptable salt thereof;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ia:

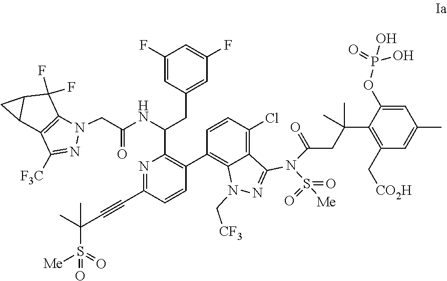 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every seven days, wherein the tablet comprises:
about 40 w/w % of the compound of Formula Ia, or a pharmaceutically acceptable salt thereof;
about 27.25 w/w % of mannitol;
about 27.25 w/w % of microcrystalline cellulose;
about 4 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ia:

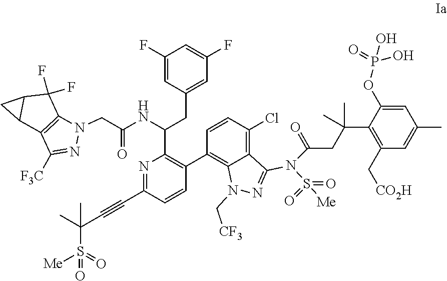 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every seven days, wherein the tablet comprises:
about 35 w/w % to about 45 w/w % of the compound of Formula Ia;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ia:

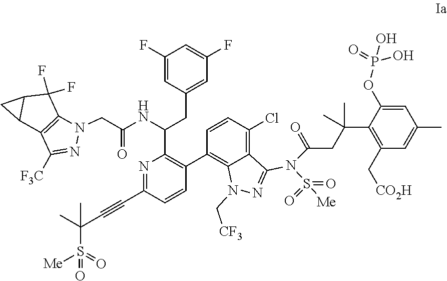 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every seven days, wherein the tablet comprises:
about 35 w/w % to about 45 w/w % of the compound of Formula Ia;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ia:

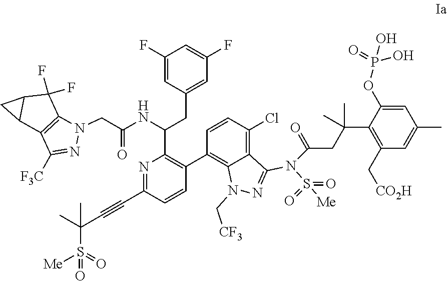 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every seven days, wherein the tablet comprises:
about 35 w/w % to about 45 w/w % of the compound of Formula Ia;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ia:

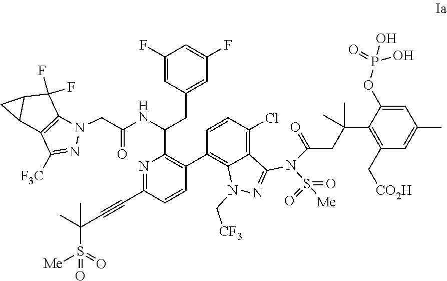 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every seven days, wherein the tablet comprises:
about 35 w/w % to about 45 w/w % of the compound of Formula Ia;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ia:

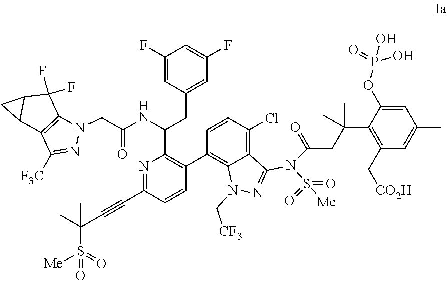 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every seven days, wherein the tablet comprises:
about 40 w/w % of the compound of Formula Ia;
about 27.25 w/w % of mannitol;
about 27.25 w/w % of microcrystalline cellulose;
about 4 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ib:

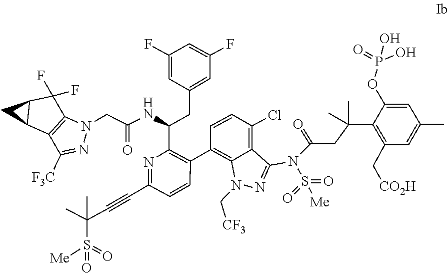 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every seven days, wherein the tablet comprises:
about 35 w/w % to about 45 w/w % of the compound of Formula Ib, or a pharmaceutically acceptable salt thereof;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib:

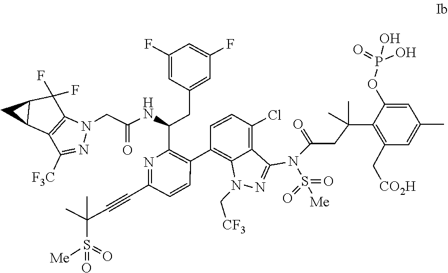 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every seven days, wherein the tablet comprises:
about 35 w/w % to about 45 w/w % of the compound of Formula Ib, or a pharmaceutically acceptable salt thereof;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the

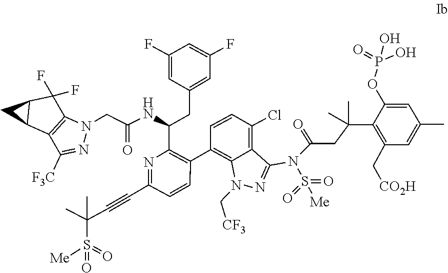 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every seven days, wherein the tablet comprises:
about 35 w/w % to about 45 w/w % of the compound of Formula Ib, or a pharmaceutically acceptable salt thereof,
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib:

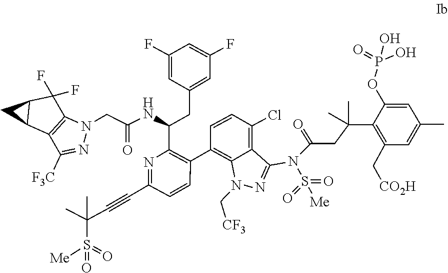 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every seven days, wherein the tablet comprises:
about 35 w/w % to about 45 w/w % of the compound of Formula Ib, or a pharmaceutically acceptable salt thereof;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib:

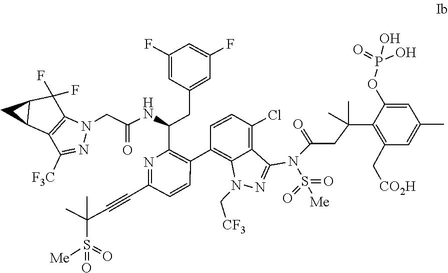 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every seven days, wherein the tablet comprises:
about 40 w/w % of the compound of Formula Ib, or a pharmaceutically acceptable salt thereof;
about 27.25 w/w % of mannitol;
about 27.25 w/w % of microcrystalline cellulose;
about 4 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ib:

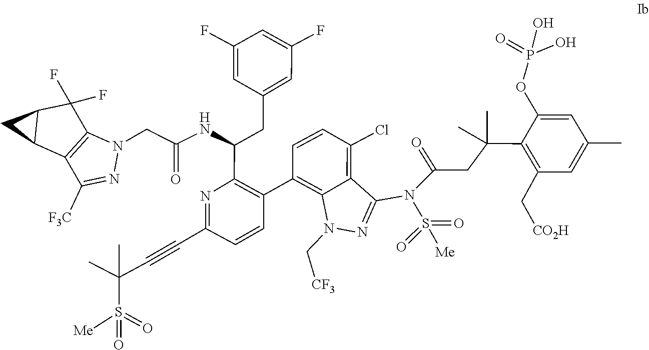 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w % to about 45 w/w % of the compound of Formula Ib;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib:

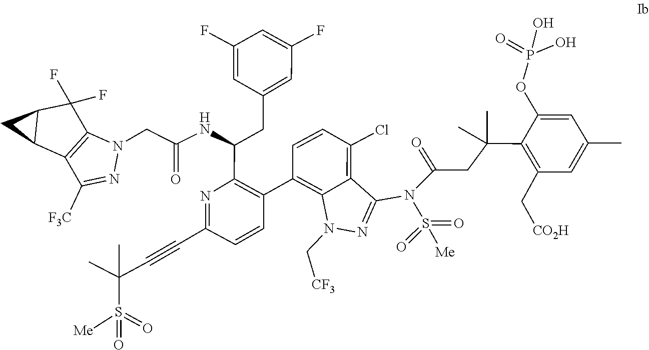 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w % to about 45 w/w % of the compound of Formula Ib;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib:

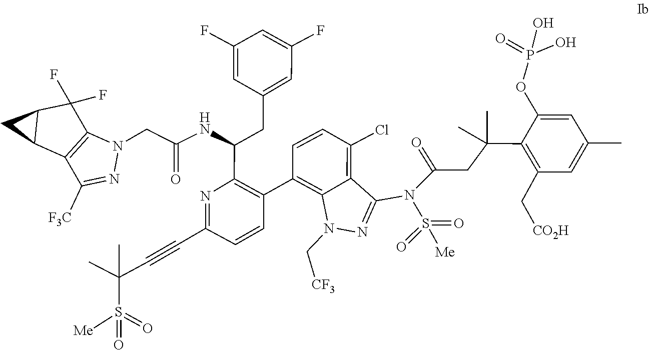 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w % to about 45 w/w % of the compound of Formula Ib;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib:

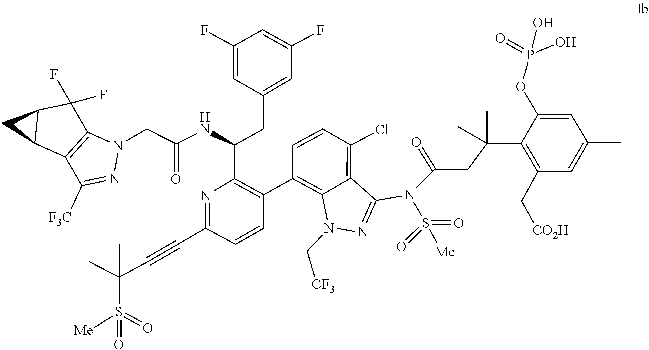 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w % to about 45 w/w % of the compound of Formula Ib;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib:

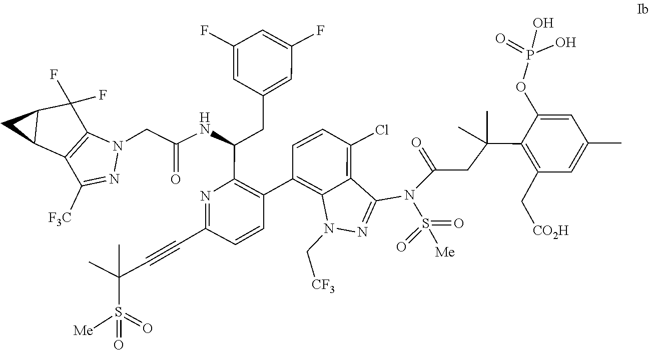 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 40 w/w % of the compound of Formula Ib;
about 27.25 w/w % of mannitol;
about 27.25 w/w % of microcrystalline cellulose;
about 4 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a crystalline form of the compound of Formula Ib:

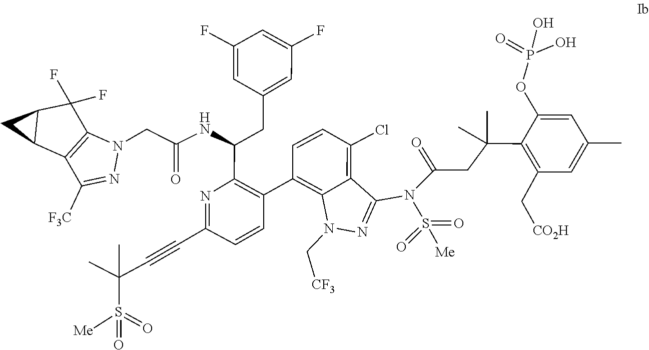 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w % to about 45 w/w % of the crystalline form of the compound of Formula Ib;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a crystalline form of the compound of Formula Ib:

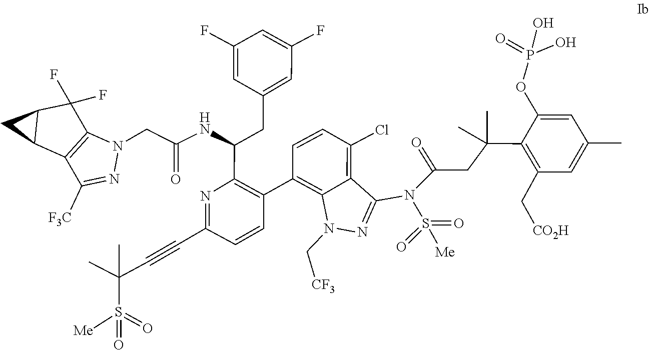 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w/% to about 45 w/w/% of the crystalline form of the compound of Formula Ib;
about 27.1 w/w/% to about 27.6 w/w/% of mannitol;
about 27.1 w/w/% to about 27.6 w/w/% of microcrystalline cellulose;
about 2 w/w/% to about 6 w/w/% of crospovidone; and
about 1 w/w/% to about 3 w/w/% of magnesium stearate.

In some embodiments, the present disclosure relates to a method of reducing risk of sexually acquired HIV in a patient, comprising orally administering to the patient a tablet comprising a crystalline form of the compound of Formula Ib:

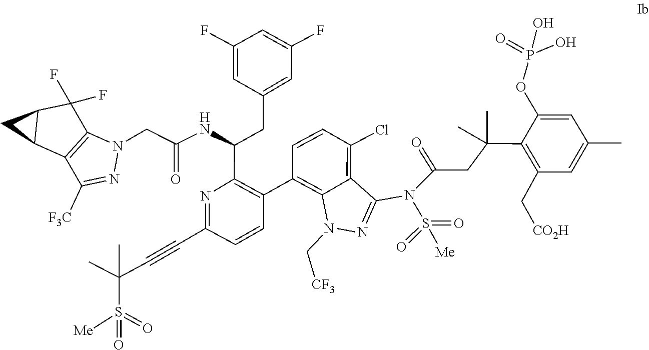 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w % to about 45 w/w % of the crystalline form of the compound of Formula Ib;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a crystalline form of the compound of Formula Ib:

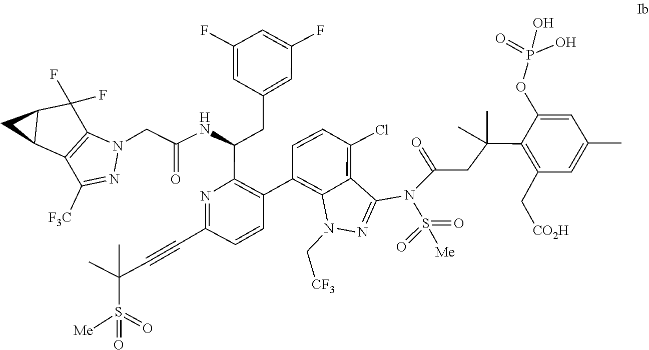 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w % to about 45 w/w % of the crystalline form of the compound of Formula Ib;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a crystalline form of the compound of Formula Ib:

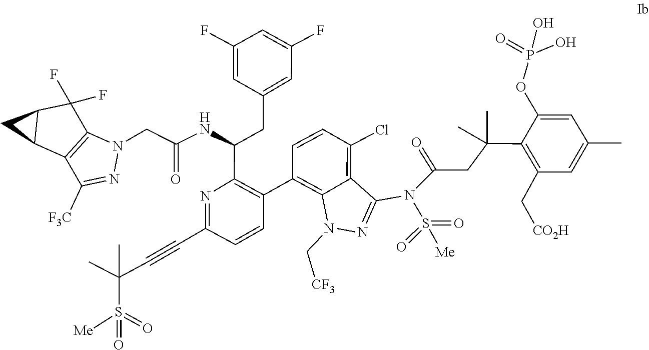 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 40 w/w % of the crystalline form of the compound of Formula Ib;
about 27.25 w/w % of mannitol;
about 27.25 w/w % of microcrystalline cellulose;
about 4 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ib, crystalline Form I:

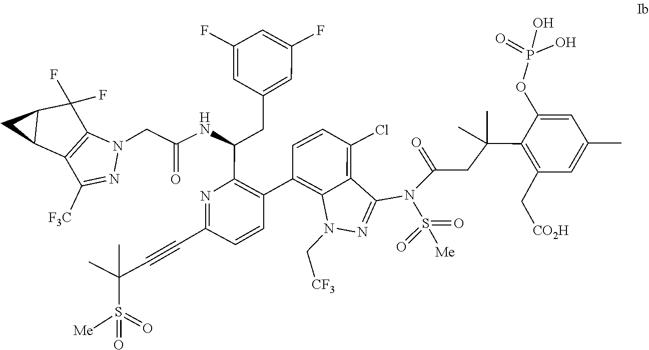 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w % to about 45 w/w % of the crystalline form of the compound of Formula Ib, crystalline Form I;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib, crystalline Form I:

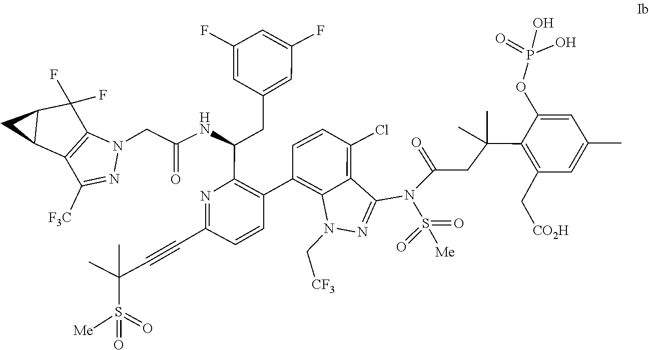 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w/% to about 45 w/w/% of the compound of Formula Ib, crystalline Form I;
about 27.1 w/w/% to about 27.6 w/w/% of mannitol;
about 27.1 w/w/% to about 27.6 w/w/% of microcrystalline cellulose;
about 2 w/w/% to about 6 w/w/% of crospovidone; and
about 1 w/w/% to about 3 w/w/% of magnesium stearate.

In some embodiments, the present disclosure relates to a method of reducing the risk of sexually acquired HIV in a patient, comprising orally administering to the patient a tablet comprising a compound of Formula Ib, crystalline Form I:

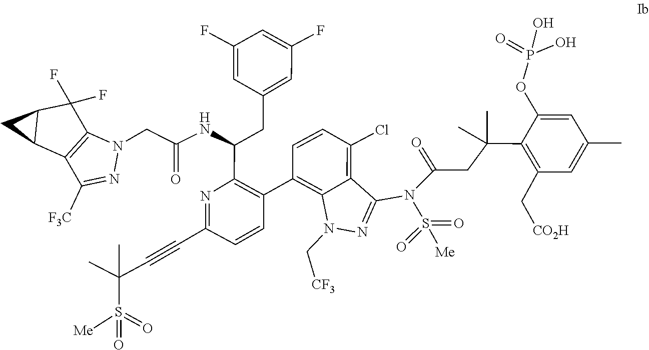 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w % to about 45 w/w % of the compound of Formula Ib, crystalline Form I;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib, crystalline Form I:

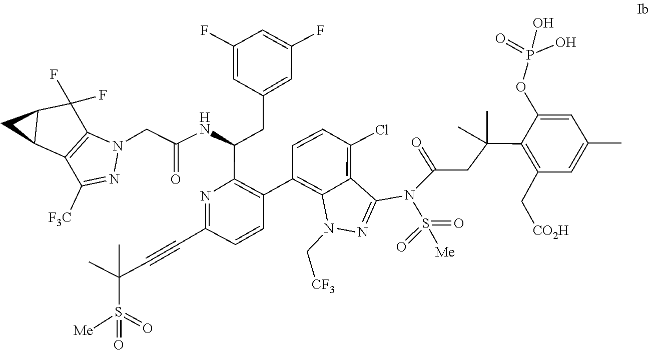 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 35 w/w % to about 45 w/w % of the compound of Formula Ib, crystalline Form I;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib, crystalline Form I:

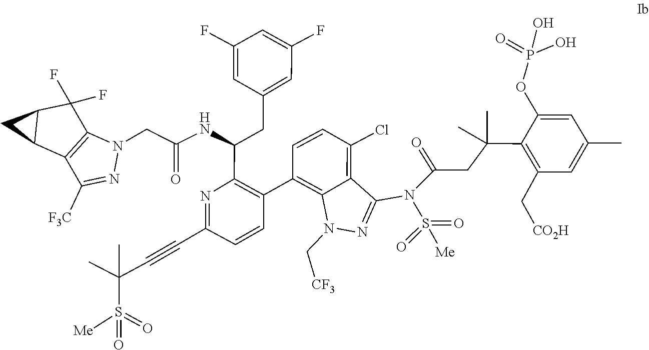 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 40 w/w % of the compound of Formula Ib, crystalline Form I;
about 27.25 w/w % of mannitol;
about 27.25 w/w % of microcrystalline cellulose;
about 4 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ia:

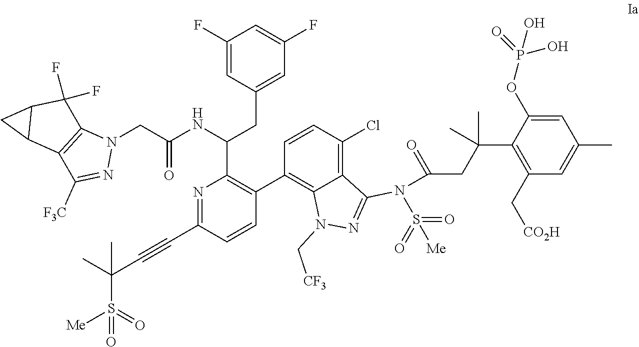 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every seven days, wherein the tablet comprises:
about 175 mg to about 225 mg of the compound of Formula Ia, or a pharmaceutically acceptable salt thereof;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the present disclosure relates to a method of reducing the risk of sexually acquired HIV in a patient, comprising orally administering to the patient a tablet comprising a compound of Formula Ia:

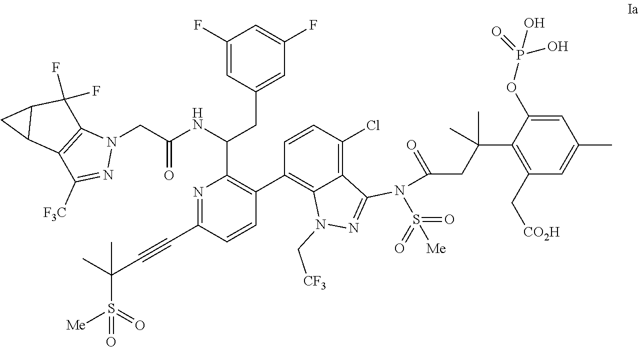 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every seven days, wherein the tablet comprises:
about 200 mg of the compound of Formula Ia, or a pharmaceutically acceptable salt thereof;
about 135 mg to about 137 mg of mannitol;
about 135 mg to about 137 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the

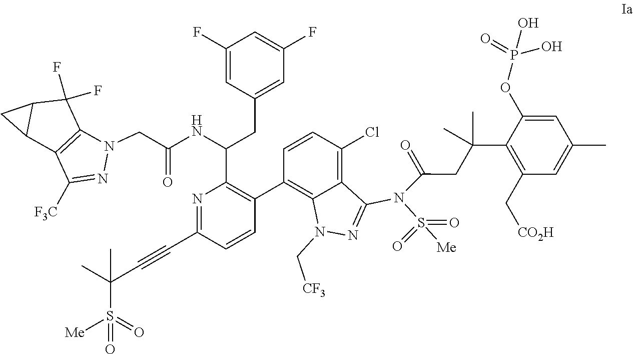 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every seven days, wherein the tablet comprises:
about 200 mg of the compound of Formula Ia, or a pharmaceutically acceptable salt thereof;
about 136.2 mg of mannitol;
about 136.2 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ia:

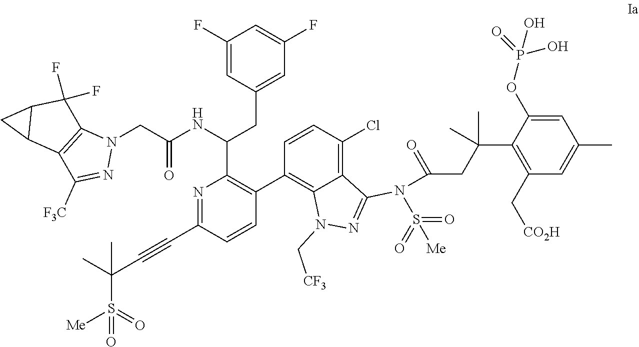 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every seven days, wherein the tablet comprises:
about 200 mg of the compound of Formula Ia, or a pharmaceutically acceptable salt thereof;
about 136.3 mg of mannitol;
about 136.3 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ia:

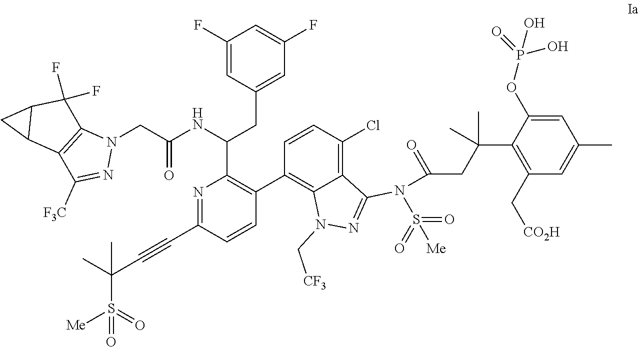 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every seven days, wherein the tablet comprises:
about 200 mg of the compound of Formula Ia, or a pharmaceutically acceptable salt thereof;
about 136.25 mg of mannitol;
about 136.25 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ia:

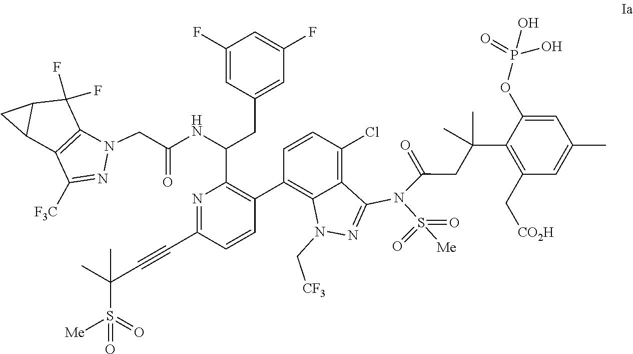 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every seven days, wherein the tablet comprises:
about 175 mg to about 225 mg of the compound of Formula Ia;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the present disclosure relates to a method of reducing the risk of sexually acquired HIV in a patient, comprising orally administering to the patient a tablet comprising a compound of Formula Ia:

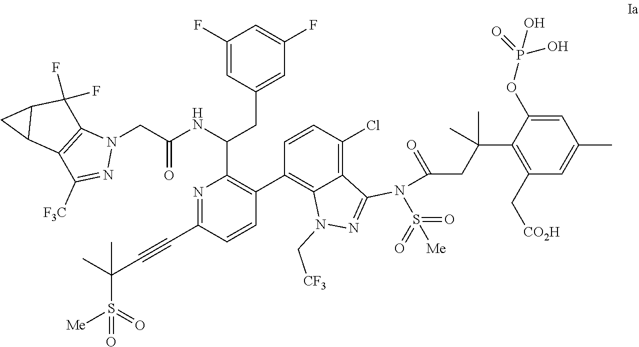 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every seven days, wherein the tablet comprises:
about 200 mg of the compound of Formula Ia;
about 135 mg to about 137 mg of mannitol;
about 135 mg to about 137 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ia:

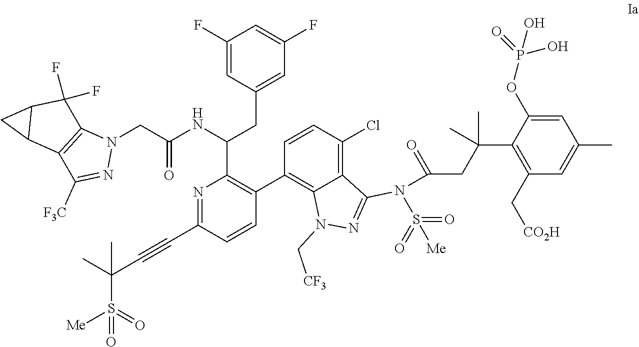 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every seven days, wherein the tablet comprises:
about 200 mg of the compound of Formula Ia;
about 136.2 mg of mannitol;
about 136.2 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ia:

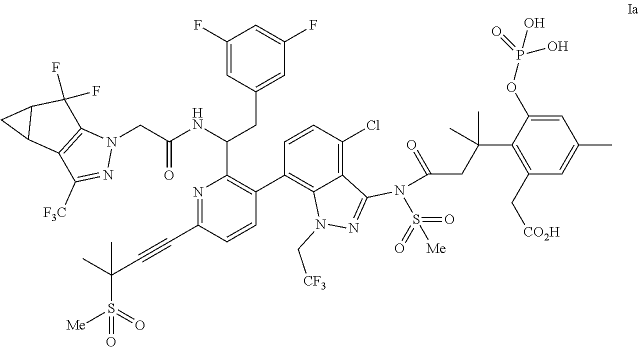 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every seven days, wherein the tablet comprises:
about 200 mg of the compound of Formula Ia;
about 136.3 mg of mannitol;
about 136.3 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ia:

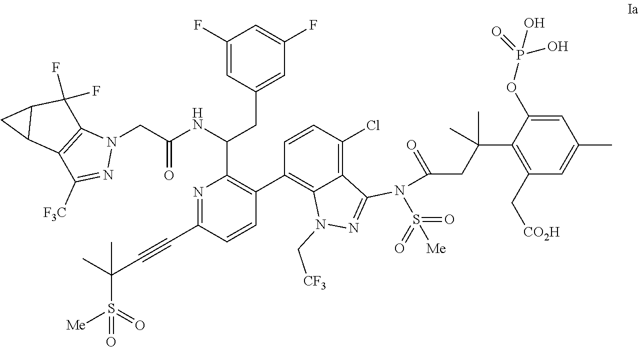 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every seven days, wherein the tablet comprises:
about 200 mg of the compound of Formula Ia;
about 136.25 mg of mannitol;
about 136.25 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib:

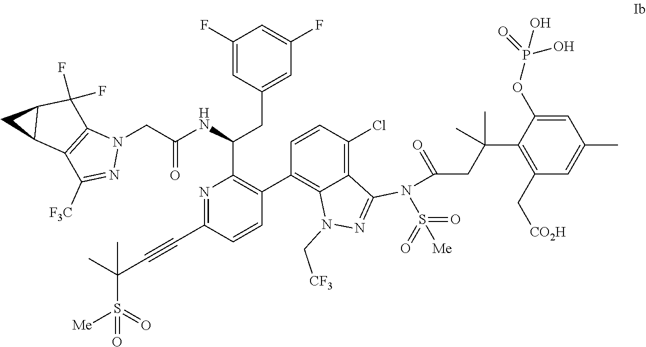 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every seven days, wherein the tablet comprises:
about 175 mg to about 225 mg of the compound of Formula Ib, or a pharmaceutically acceptable salt thereof;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the present disclosure relates to a method of reducing the risk of sexually acquired HIV in a patient, comprising orally administering to the patient a tablet comprising a compound of Formula Ib:

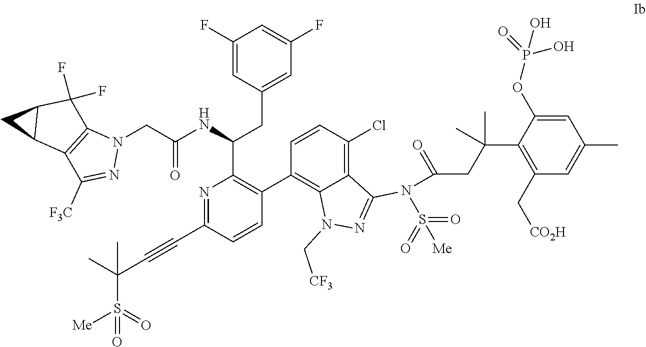 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every seven days, wherein the tablet comprises:
about 200 mg of the compound of Formula Ib, or a pharmaceutically acceptable salt thereof;
about 135 mg to about 137 mg of mannitol;
about 135 mg to about 137 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the

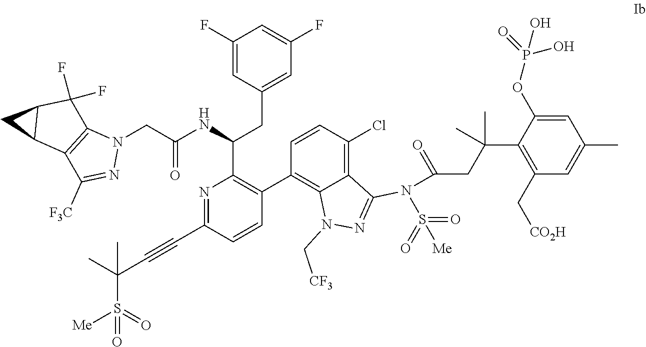 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every seven days, wherein the tablet comprises:
about 200 mg of the compound of Formula Ib, or a pharmaceutically acceptable salt thereof;
about 136.2 mg of mannitol;
about 136.2 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib:

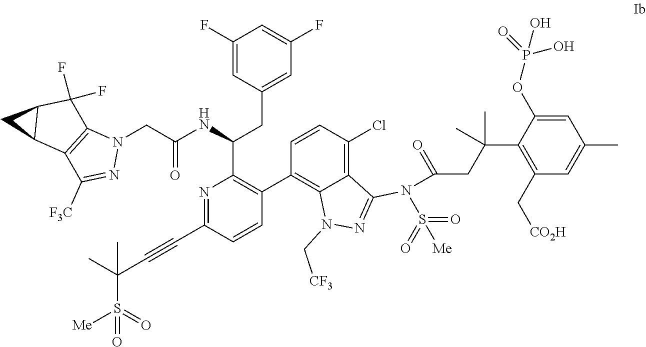 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every seven days, wherein the tablet comprises:
about 200 mg of the compound of Formula Ib, or a pharmaceutically acceptable salt thereof;
about 136.3 mg of mannitol;
about 136.3 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib:

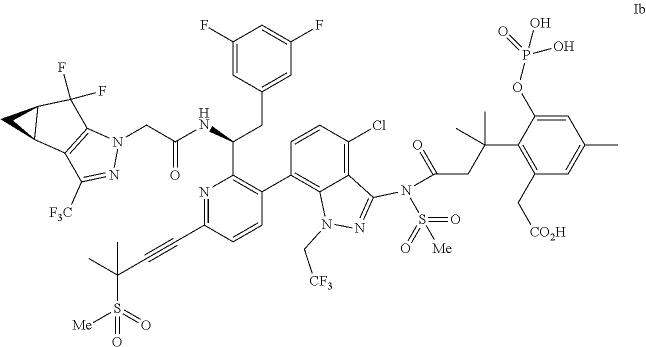 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every seven days, wherein the tablet comprises:
about 200 mg of the compound of Formula Ib, or a pharmaceutically acceptable salt thereof;
about 136.25 mg of mannitol;
about 136.25 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib:

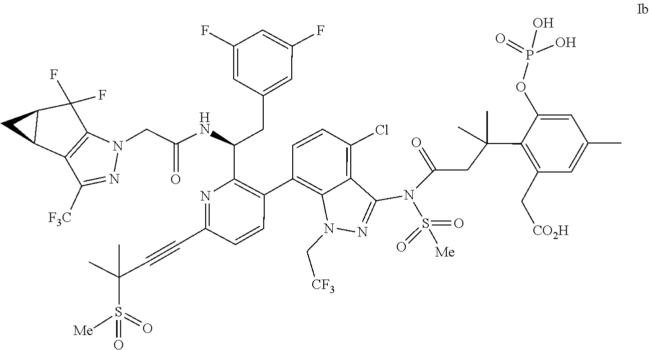 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises: about 175 mg to about 225 mg of the compound of Formula Ib;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the present disclosure relates to a method of reducing the risk of sexually acquired HIV in a patient, comprising orally administering to the patient a tablet comprising a compound of Formula Ib:

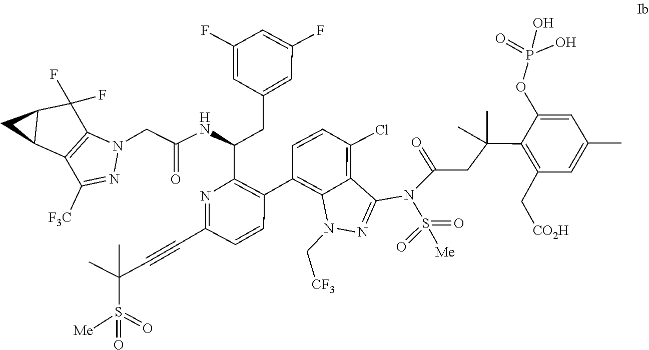 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises: about 200 mg of the compound of Formula Ib;
about 135 mg to about 137 mg of mannitol;
about 135 mg to about 137 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib:

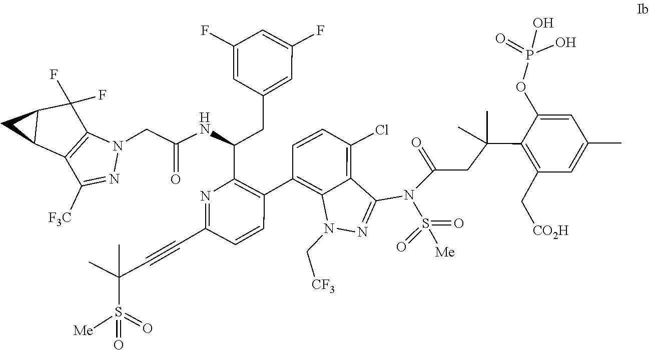 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the compound of Formula Ib;
about 136.2 mg of mannitol;
about 136.2 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib:

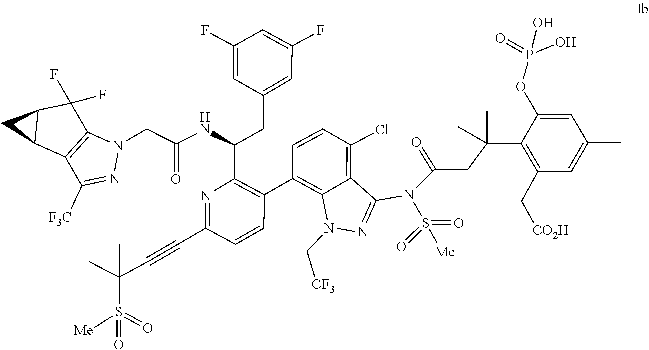 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the compound of Formula Ib;
about 136.3 mg of mannitol;
about 136.3 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib:

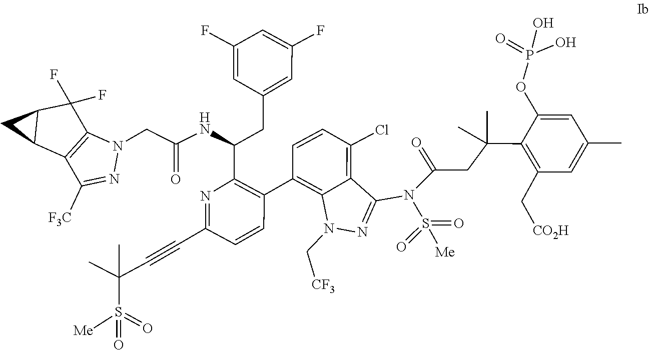 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the compound of Formula Ib;
about 136.25 mg of mannitol;
about 136.25 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a crystalline form of the compound of Formula Ib:

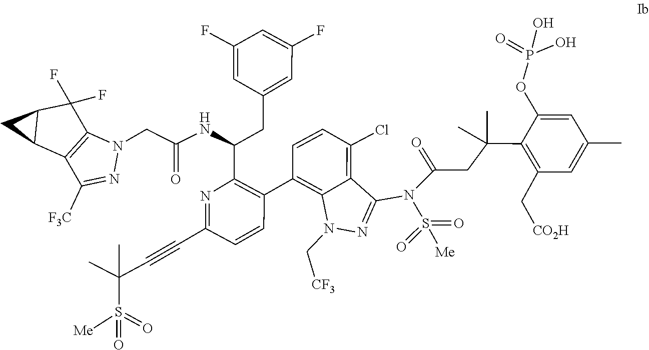 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 175 mg to about 225 mg of the crystalline form of the compound of Formula Ib;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the present disclosure relates to a method of reducing the risk of sexually acquired HIV in a patient, comprising orally administering to the patient a tablet comprising a crystalline form of the compound of Formula Ib:

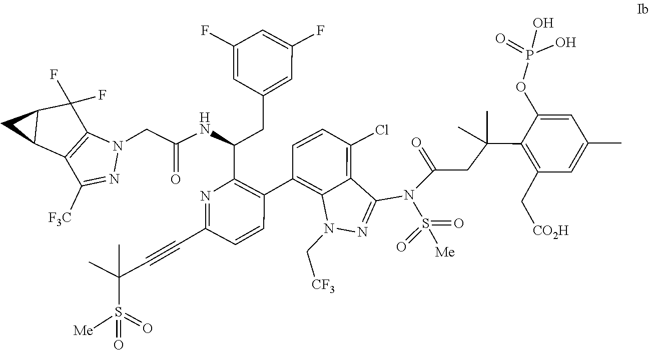 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the crystalline form of the compound of Formula Ib;
about 135 mg to about 137 mg of mannitol;
about 135 mg to about 137 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a crystalline form of the compound of Formula Ib:

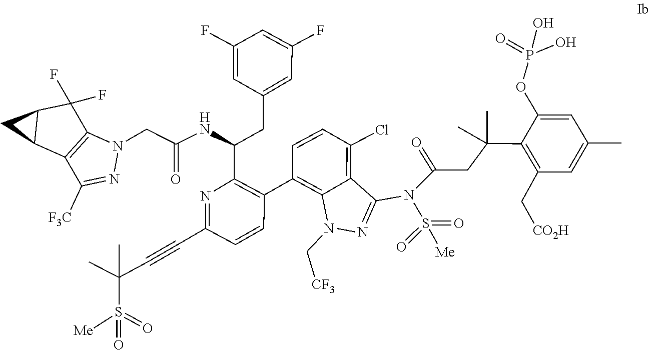 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the crystalline form of the compound of Formula Ib;
about 136.2 mg of mannitol;
about 136.2 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a crystalline form of the compound of Formula Ib:

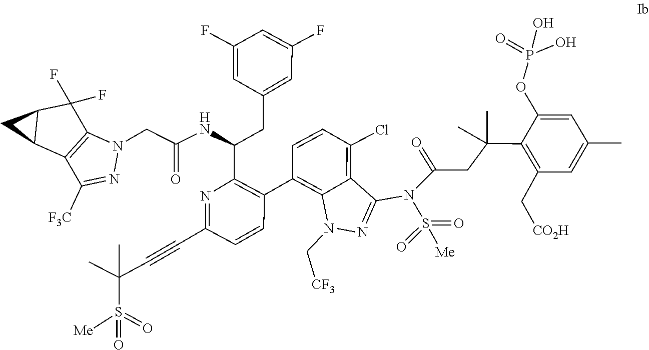 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the crystalline form of the compound of Formula Ib;
about 136.3 mg of mannitol;
about 136.3 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a crystalline form of the compound of Formula Ib:

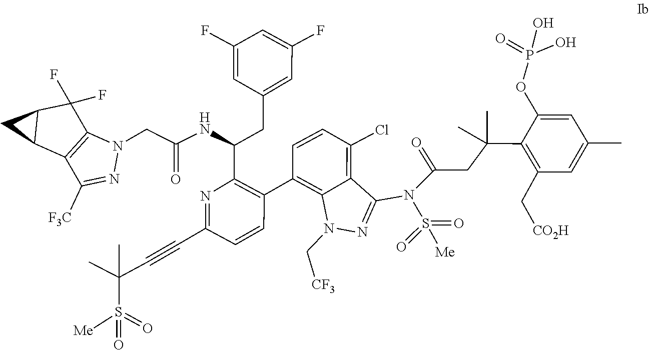 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the crystalline form of the compound of Formula Ib;
about 136.25 mg of mannitol;
about 136.25 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib, crystalline Form I:

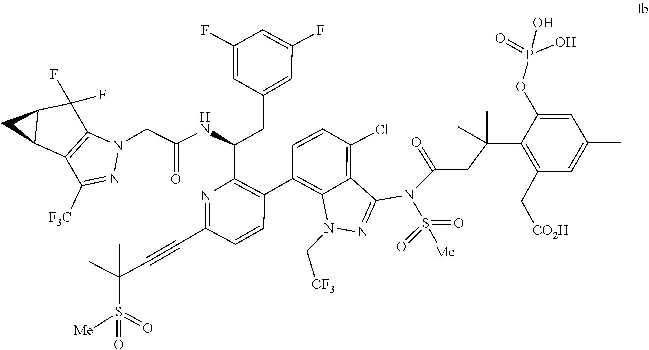 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 175 mg to about 225 mg of the compound of Formula Ib, crystalline Form I;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate.

In some embodiments, the present disclosure relates to a method of reducing the risk of sexually acquired HIV in a patient, comprising orally administering to the patient a tablet comprising a compound of Formula Ib, crystalline Form I:

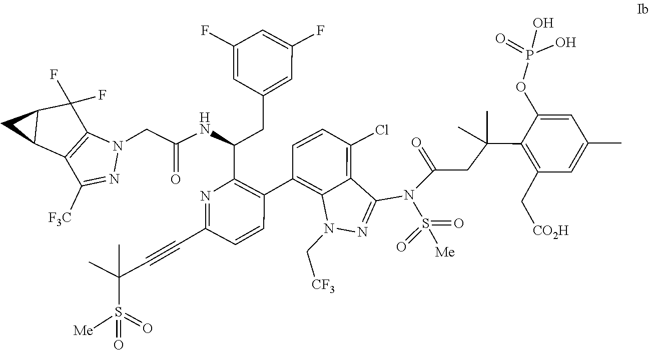 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the compound of Formula Ib, crystalline Form I;
about 135 mg to about 137 mg of mannitol;
about 135 mg to about 137 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib, crystalline Form I:

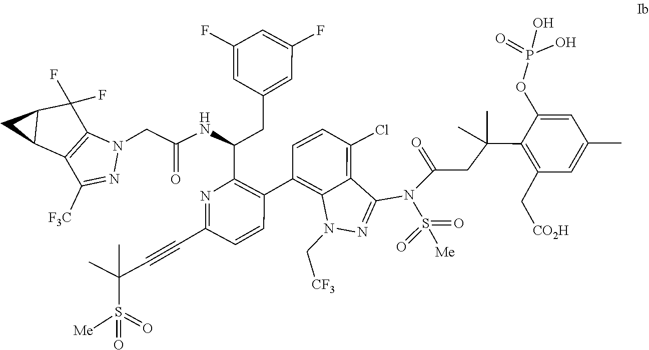 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the compound of Formula Ib, crystalline Form I;
about 136.2 mg of mannitol;
about 136.2 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib, crystalline Form I:

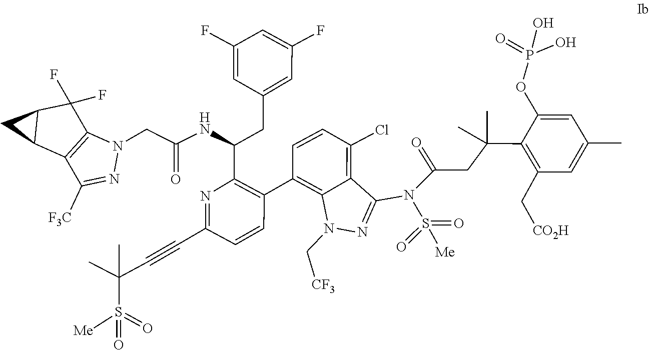 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the compound of Formula Ib, crystalline Form I;
about 136.3 mg of mannitol;
about 136.3 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ib, crystalline Form I:

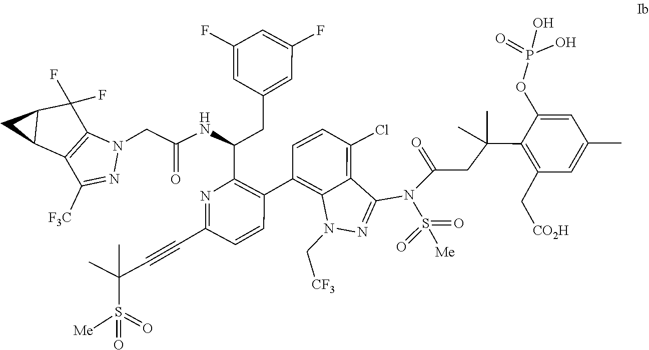 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every seven days, wherein the tablet comprises:
about 200 mg of the compound of Formula b crystalline Form I;
about 136.25 mg of mannitol;
about 136.25 mg of microcrystalline cellulose;
about 20 mg of crospovidone; and
about 7.5 mg of magnesium stearate.

In some embodiments, the present disclosure relates to a method of reducing the risk of sexually acquired HIV in a patient, comprising orally administering to the patient a tablet comprising a compound of Formula Ia:

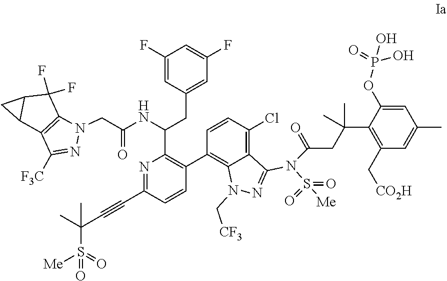 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every month, wherein the tablet comprises:
about 70 w/w % to about 80 w/w % of the compound of Formula Ia, or a pharmaceutically acceptable salt thereof;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the present disclosure relates to a method of reducing the risk of sexually acquired HIV in a patient, comprising orally administering to the patient a tablet comprising a compound of Formula Ia:

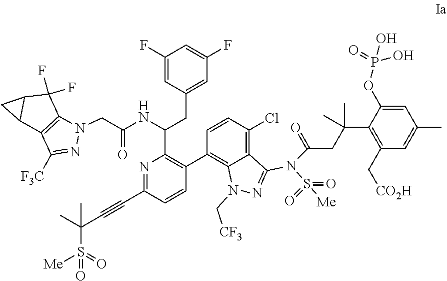 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every month, wherein the tablet comprises:
about 70 w/w % to about 80 w/w % of the compound of Formula Ia, or a pharmaceutically acceptable salt thereof;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ia:

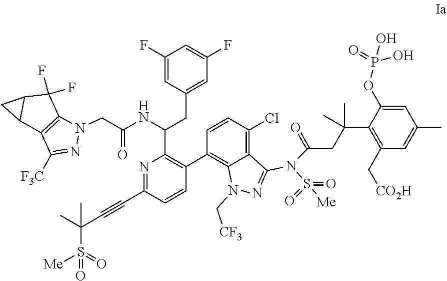 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every month, wherein the tablet comprises:
about 75 w/w % of the compound of Formula Ia, or a pharmaceutically acceptable salt thereof;
about 7.8 w/w % of mannitol;
about 7.8 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ia:

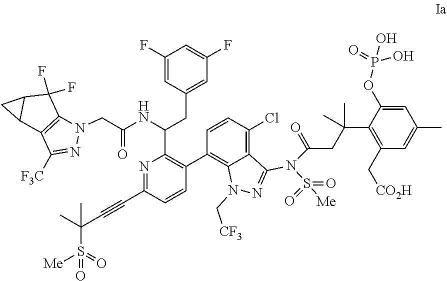 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every month, wherein the tablet comprises:
about 75 w/w % of the compound of Formula Ia, or a pharmaceutically acceptable salt thereof;
about 7.75 w/w % of mannitol;
about 7.75 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ia:

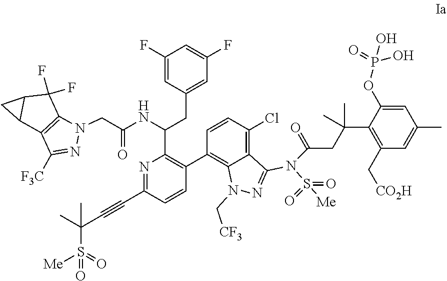 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every month, wherein the tablet comprises:
about 70 w/w % to about 80 w/w % of the compound of Formula Ia;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the present disclosure relates to a method of reducing the risk of sexually acquired HIV in a patient, comprising orally administering to the patient a tablet comprising a compound of Formula Ia:

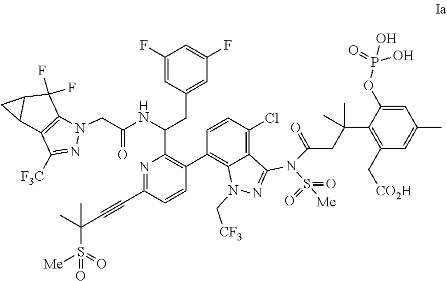 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every month, wherein the tablet comprises:
about 70 w/w % to about 80 w/w % of the compound of Formula Ia;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ia:

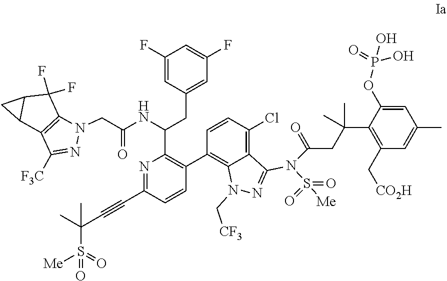 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every month, wherein the tablet comprises:
about 75 w/w % of the compound of Formula Ia;
about 7.8 w/w % of mannitol;
about 7.8 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ia:

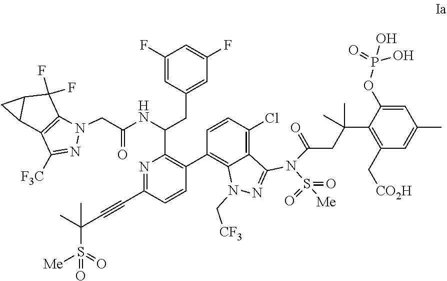 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every month, wherein the tablet comprises:
about 75 w/w % of the compound of Formula Ia;
about 7.75 w/w % of mannitol;
about 7.75 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ib:

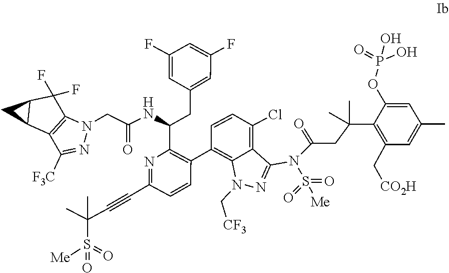 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every month, wherein the tablet comprises:
about 70 w/w % to about 80 w/w % of the compound of Formula Ib, or a pharmaceutically acceptable salt thereof;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the present disclosure relates to a method of reducing the risk of sexually acquired HIV in a patient, comprising orally administering to the patient a tablet comprising a compound of Formula Ib:

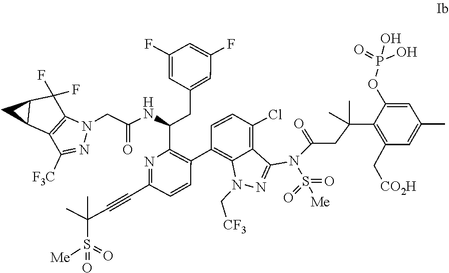 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every month, wherein the tablet comprises:
about 70 w/w % to about 80 w/w % of the compound of Formula Ib, or a pharmaceutically acceptable salt thereof;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib:

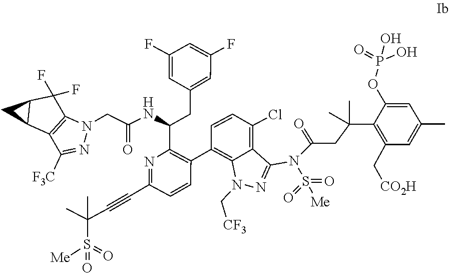 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every month, wherein the tablet comprises:
about 75 w/w % of the compound of Formula Ib, or a pharmaceutically acceptable salt thereof;
about 7.8 w/w % of mannitol;
about 7.8 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ib:

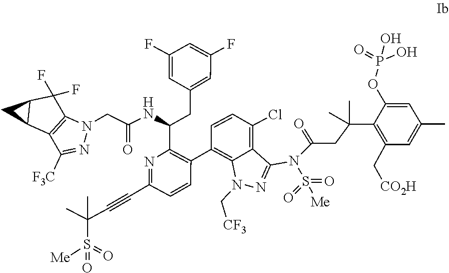 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every month, wherein the tablet comprises:
about 75 w/w % of the compound of Formula Ib, or a pharmaceutically acceptable salt thereof;
about 7.75 w/w % of mannitol;
about 7.75 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ib:

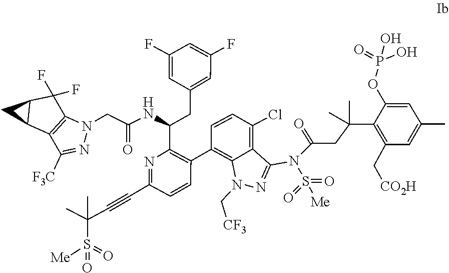 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 70 w/w % to about 80 w/w % of the compound of Formula Ib;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the present disclosure relates to a method of reducing the risk of sexually acquired HIV in a patient, comprising orally administering to the patient a tablet comprising a compound of Formula Ib:

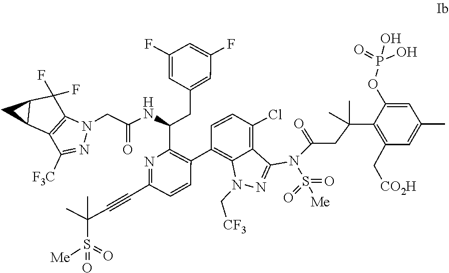 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 70 w/w % to about 80 w/w % of the compound of Formula Ib;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a compound of Formula Ib:

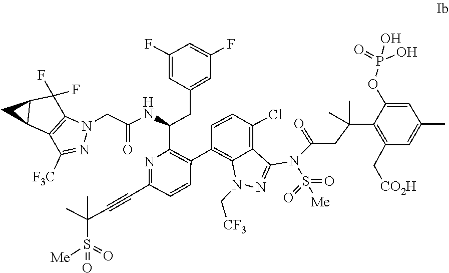 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 75 w/w/% of the compound of Formula Ib;
about 7.8 w/w/% of mannitol;
about 7.8 w/w/% of microcrystalline cellulose;
about 8 w/w/% of crospovidone; and
about 1.5 w/w/% of magnesium stearate.

In some embodiments, the present disclosure relates to a method of reducing the risk of sexually acquired HIV in a patient, comprising orally administering to the patient a tablet comprising a compound of Formula Ib:

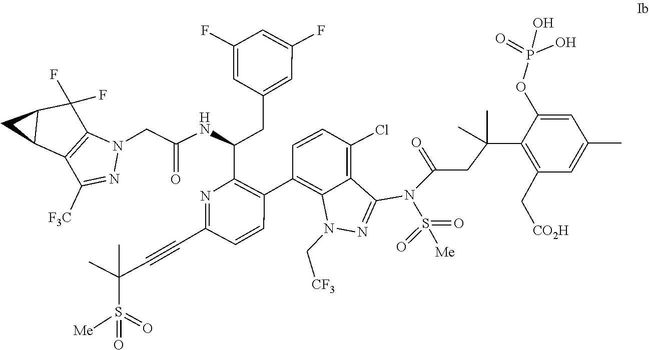 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 75 w/w % of the compound of Formula Ib;
about 7.75 w/w % of mannitol;
about 7.75 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a crystalline form of the compound of Formula Ib:

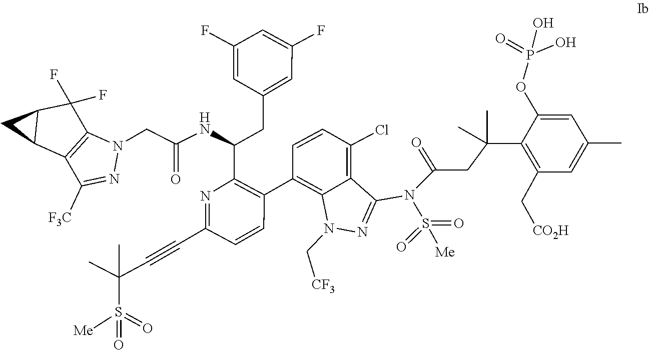 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 70 w/w % to about 80 w/w % of the crystalline form of the compound of Formula Ib;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the present disclosure relates to a method of reducing the risk of sexually acquired HIV in a patient, comprising orally administering to the patient a tablet comprising a crystalline form of the compound of Formula Ib:

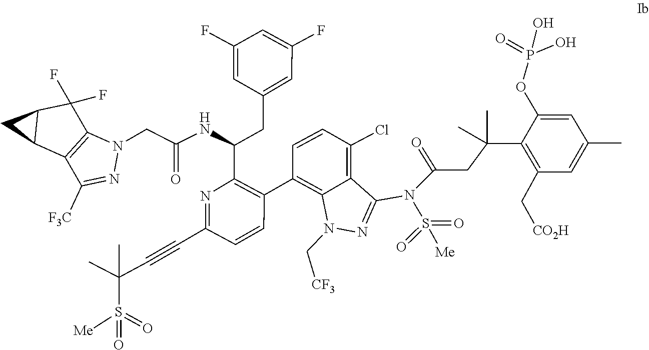 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 70 w/w % to about 80 w/w % of the crystalline form of the compound of Formula Ib;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate.

In some embodiments, the present disclosure relates to a method of reducing the risk of sexually acquired HIV in a patient, comprising orally administering to the patient a tablet comprising a crystalline form of the compound of Formula Ib:

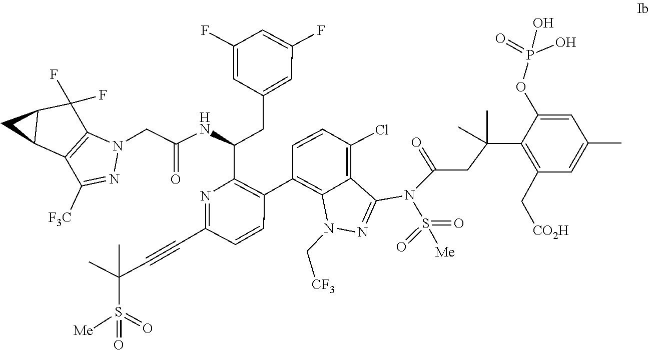 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 75 w/w % of the crystalline form of the compound of Formula Ib;
about 7.8 w/w % of mannitol;
about 7.8 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a crystalline form of the compound of Formula Ib:

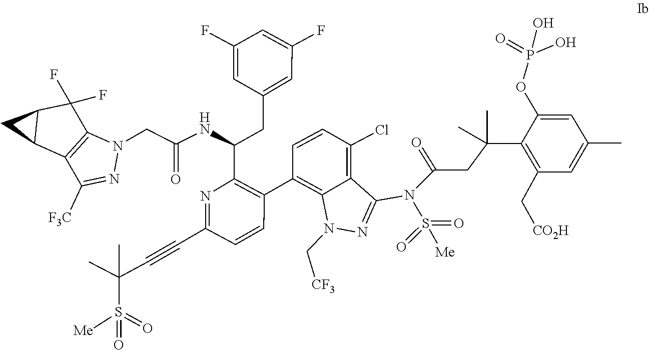 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 75 w/w % of the crystalline form of the compound of Formula Ib;
about 7.75 w/w % of mannitol;
about 7.75 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ib, crystalline Form I:

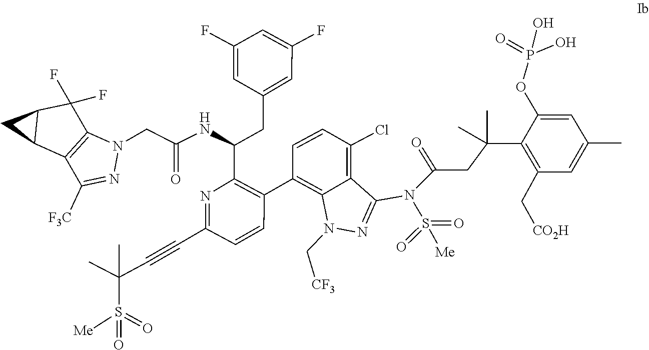 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 70 w/w % to about 80 w/w % of the compound of Formula Ib, crystalline Form I;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate.

In some embodiments, the present disclosure relates to a method of reducing the risk of sexually acquired HIV in a patient, comprising orally administering to the patient a tablet comprising a compound of Formula Ib, crystalline Form I:

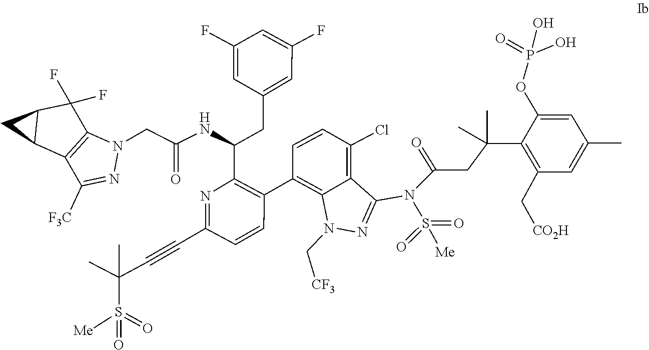 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 70 w/w/% to about 80 w/w/% of the compound of Formula Ib, crystalline Form I;
about 6 w/w/% to about 9 w/w/% of mannitol;
about 6 w/w/% to about 9 w/w/% of microcrystalline cellulose;
about 6 w/w/% to about 9 w/w/% of crospovidone; and
about 1 w/w/% to about 3 w/w/% of magnesium stearate.

In some embodiments, the present disclosure relates to a method of reducing the risk of sexually acquired HIV in a patient, comprising orally administering to the patient a tablet comprising a compound of Formula Ib, crystalline Form I:

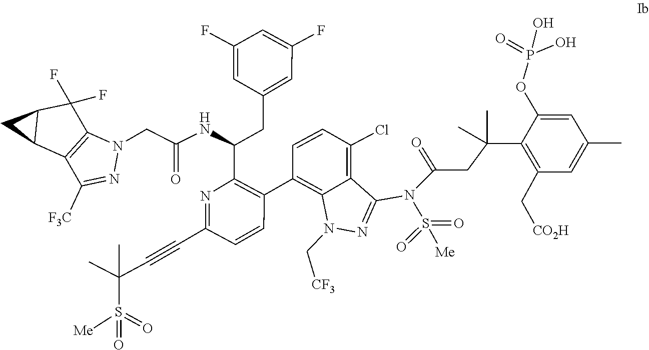 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 75 w/w % of the compound of Formula Ib, crystalline Form I;
about 7.8 w/w % of mannitol;
about 7.8 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ib, crystalline Form I:

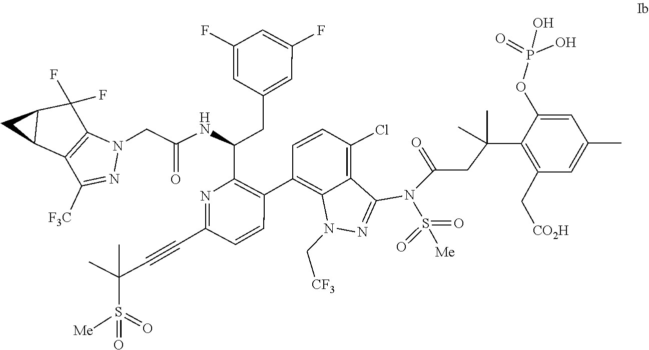 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 75 w/w % of the compound of Formula Ib, crystalline Form I;
about 7.75 w/w % of mannitol;
about 7.75 w/w % of microcrystalline cellulose;
about 8 w/w % of crospovidone; and
about 1.5 w/w % of magnesium stearate.

In some embodiments, the present disclosure relates to a method of reducing the risk of sexually acquired HIV in a patient, comprising orally administering to the patient a tablet comprising a compound of Formula Ia:

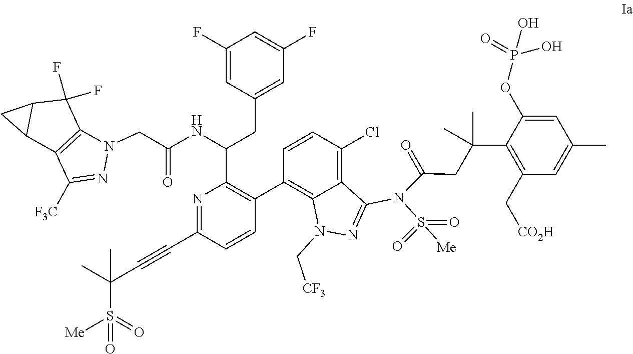 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every month, wherein the tablet comprises:
about 1000 mg to about 1500 mg of the compound of Formula Ia, or a pharmaceutically acceptable salt thereof;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the present disclosure relates to a method of reducing the risk of sexually acquired HIV in a patient, comprising orally administering to the patient a tablet comprising a compound of Formula Ia:

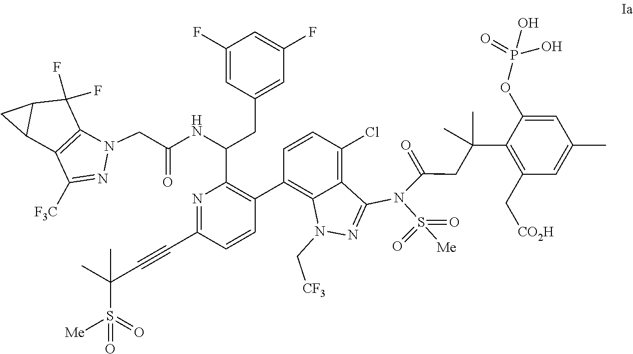 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every month, wherein the tablet comprises:
about 1100 mg to about 1300 mg of the compound of Formula Ia, or a pharmaceutically acceptable salt thereof;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the present disclosure relates to a method of reducing the risk of sexually acquired HIV in a patient, comprising orally administering to the patient a tablet comprising a compound of Formula Ia:

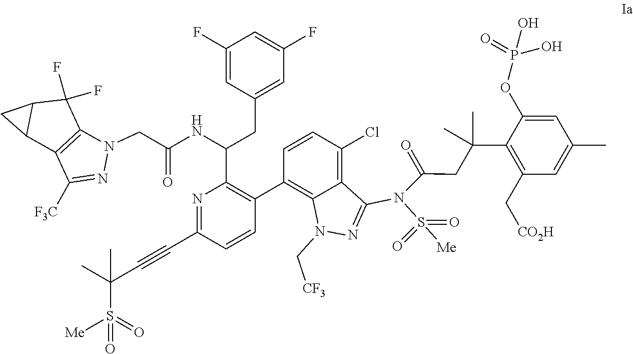 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

or a pharmaceutically acceptable salt thereof, once every month, wherein the tablet comprises:
about 1200 mg of the compound of Formula Ia, or a pharmaceutically acceptable salt thereof;
about 124 mg of mannitol;
about 124 mg of microcrystalline cellulose;
about 128 mg of crospovidone;
about 24 mg of magnesium stearate.

In some embodiments, the present disclosure relates to a method of reducing the risk of sexually acquired HIV in a patient, comprising orally administering to the patient a tablet comprising a compound of Formula Ia:

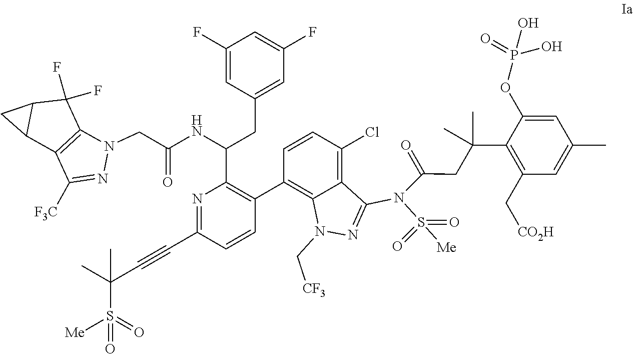 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every month, wherein the tablet comprises:
about 1000 mg to about 1500 mg of the compound of Formula Ia;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the present disclosure relates to a method of reducing the risk of sexually acquired HIV in a patient, comprising orally administering to the patient a tablet comprising a compound of Formula Ia:

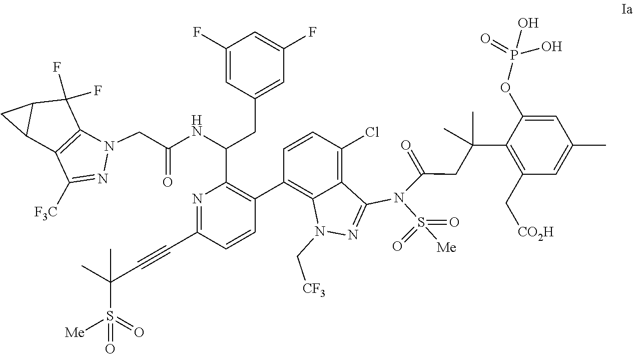 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every month, wherein the tablet comprises:
about 1100 mg to about 1300 mg of the compound of Formula Ia;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the present disclosure relates to a method of reducing the risk of sexually acquired HIV in a patient, comprising orally administering to the patient a tablet comprising a compound of Formula Ia:

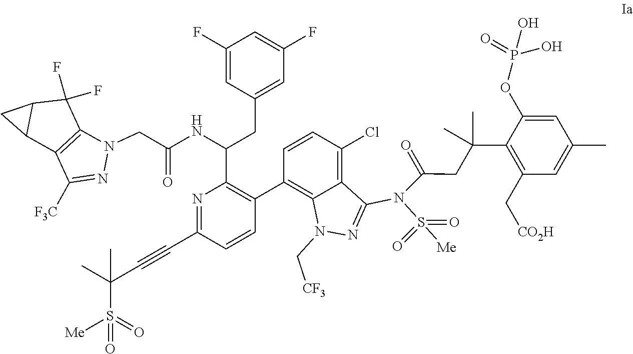 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]

once every month, wherein the tablet comprises:
about 1200 mg of the compound of Formula Ia;
about 124 mg of mannitol;
about 124 mg of microcrystalline cellulose;
about 128 mg of crospovidone;
about 24 mg of magnesium stearate.

In some embodiments, the present disclosure relates to a method of reducing the risk of sexually acquired HIV in a patient, comprising orally administering to the patient a tablet comprising a compound of Formula Ib:

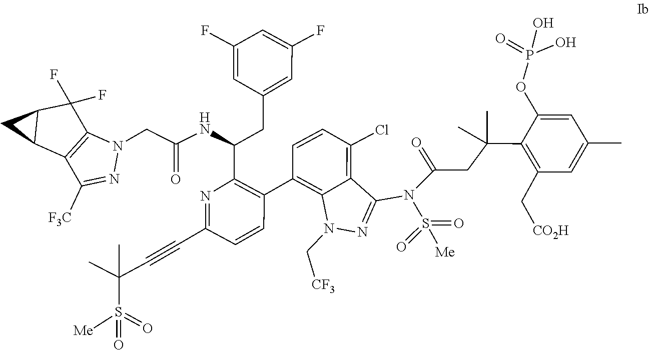 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every month, wherein the tablet comprises:
about 1000 mg to about 1500 mg of the compound of Formula Ib, or a pharmaceutically acceptable salt thereof;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the present disclosure relates to a method of reducing the risk of sexually acquired HIV in a patient, comprising orally administering to the patient a tablet comprising a compound of Formula Ib:

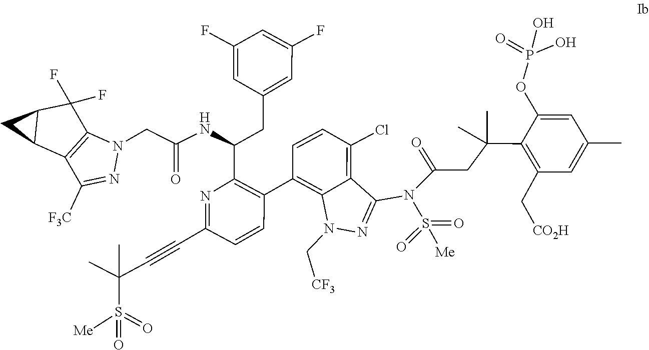 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every month, wherein the tablet comprises:
about 1100 mg to about 1300 mg of the compound of Formula Ib, or a pharmaceutically acceptable salt thereof,
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the present disclosure relates to a method of reducing the risk of sexually acquired HIV in a patient, comprising orally administering to the patient a tablet comprising a compound of Formula Ib:

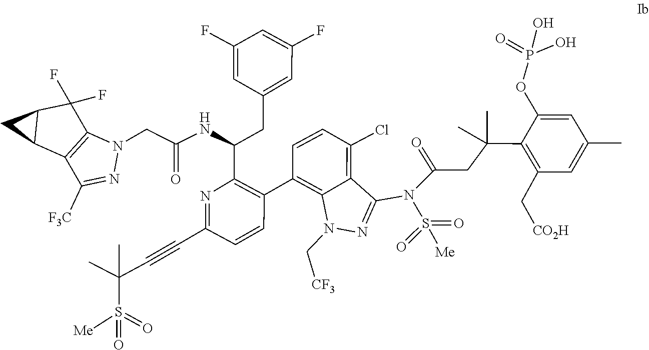 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

or a pharmaceutically acceptable salt thereof, once every month, wherein the tablet comprises:
about 1200 mg of the compound of Formula Ib, or a pharmaceutically acceptable salt thereof;
about 124 mg of mannitol;
about 124 mg of microcrystalline cellulose;
about 128 mg of crospovidone;
about 24 mg of magnesium stearate.

In some embodiments, the present disclosure relates to a method of reducing the risk of sexually acquired HIV in a patient, comprising orally administering to the patient a tablet comprising a compound of Formula Ib:

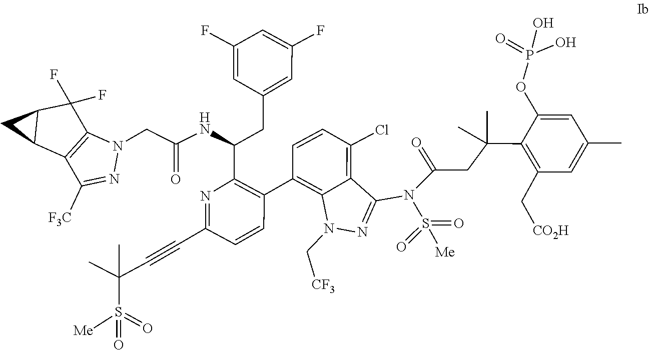 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 1000 mg to about 1500 mg of the compound of Formula Ib;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the present disclosure relates to a method of reducing the risk of sexually acquired HIV in a patient, comprising orally administering to the patient a tablet comprising a compound of Formula Ib:

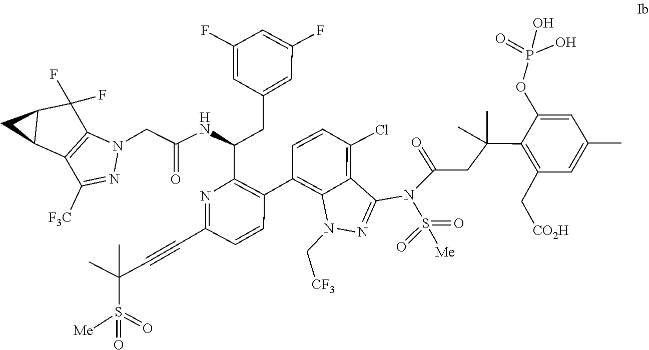 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 1100 mg to about 1300 mg of the compound of Formula Ib;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the present disclosure relates to a method of reducing the risk of sexually acquired HIV in a patient, comprising orally administering to the patient a tablet comprising a compound of Formula Ib:

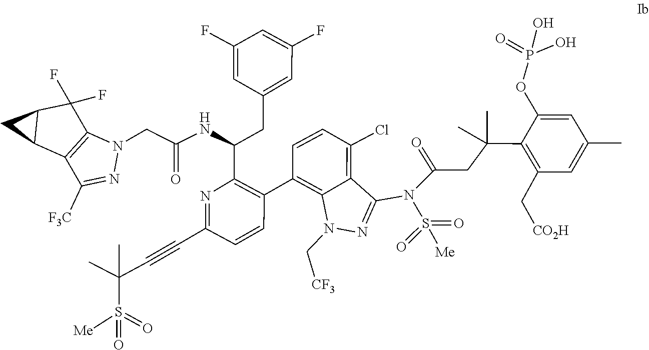 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 1200 mg of the compound of Formula Ib;
about 124 mg of mannitol;
about 124 mg of microcrystalline cellulose;
about 128 mg of crospovidone;
about 24 mg of magnesium stearate.

In some embodiments, the present disclosure relates to a method of reducing the risk of sexually acquired HIV in a patient, comprising orally administering to the patient a tablet comprising a crystalline form of the compound of Formula Ib:

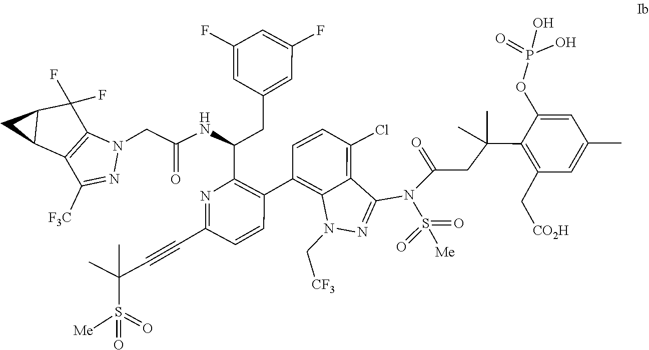 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 1000 mg to about 1500 mg of the crystalline form of the compound of Formula Ib;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the present disclosure relates to a method of reducing the risk of sexually acquired HIV in a patient, comprising orally administering to the patient a tablet comprising a crystalline form of the compound of Formula Ib:

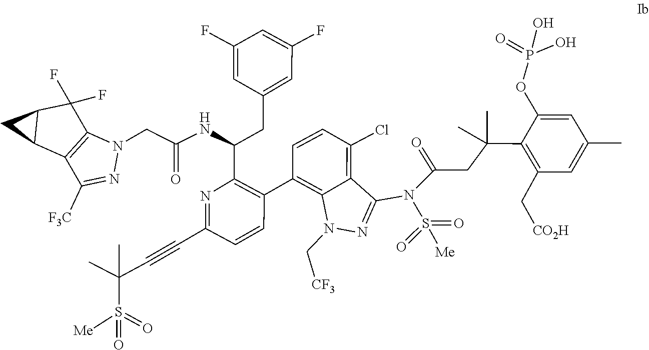 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 1100 mg to about 1300 mg of the crystalline form of the compound of Formula Ib;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the present disclosure relates to a method of reducing the risk of sexually acquired HIV in a patient, comprising orally administering to the patient a tablet comprising a crystalline form of the compound of Formula Ib:

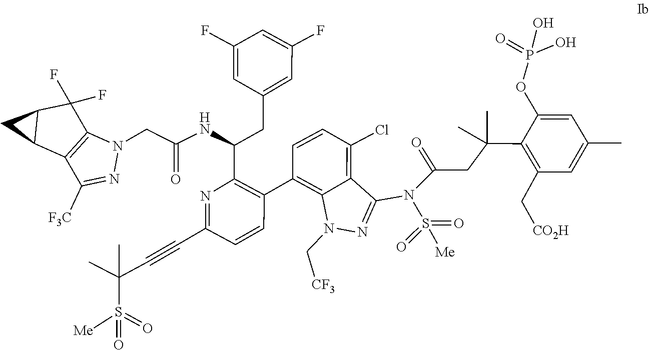 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 1200 mg of the crystalline form of the compound of Formula Ib;
about 124 mg of mannitol;
about 124 mg of microcrystalline cellulose;
about 128 mg of crospovidone;
about 24 mg of magnesium stearate.

In some embodiments, the present disclosure relates to a method of reducing the risk of sexually acquired HIV in a patient, comprising orally administering to the patient a tablet comprising a compound of Formula Ib, crystalline Form I:

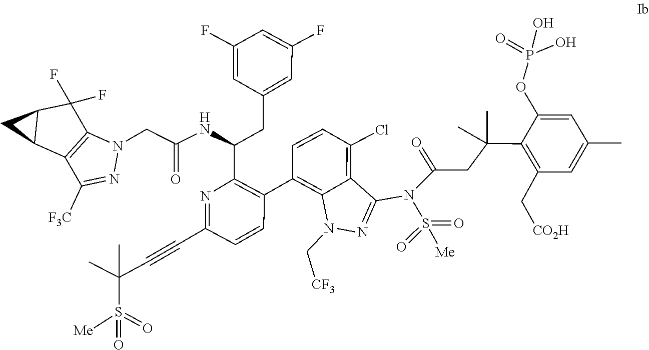 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 1000 mg to about 1500 mg of the compound of Formula Ib, crystalline Form I;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate.

In some embodiments, the present disclosure relates to a method of reducing the risk of sexually acquired HIV in a patient, comprising orally administering to the patient a tablet comprising a compound of Formula Ib, crystalline Form I:

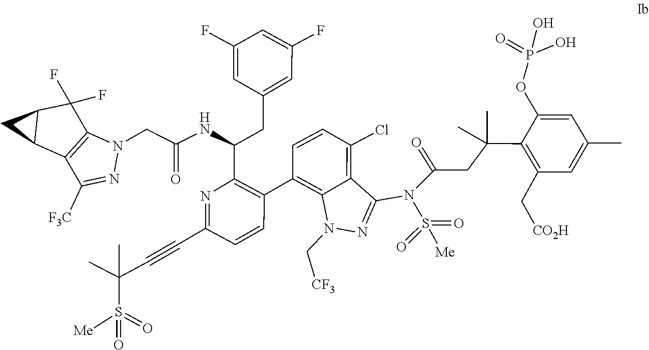 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 1100 mg to about 1300 mg of the compound of Formula Ib, crystalline Form I;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate.

In some embodiments, the present disclosure relates to a method of reducing the risk of sexually acquired HIV in a patient, comprising orally administering to the patient a tablet comprising a compound of Formula Ib, crystalline Form I:

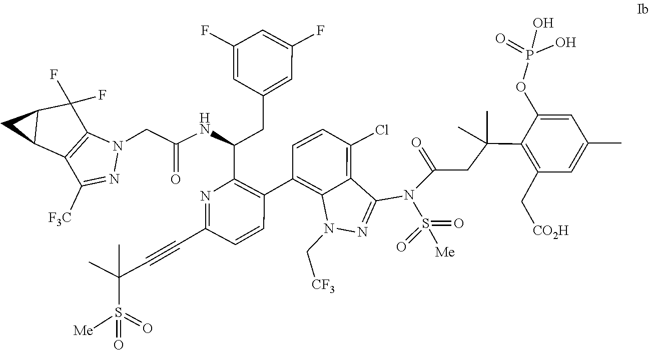 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

once every month, wherein the tablet comprises:
about 1200 mg of the compound of Formula Ib, crystalline Form I;
about 124 mg of mannitol;
about 124 mg of microcrystalline cellulose;
about 128 mg of crospovidone;
about 24 mg of magnesium stearate.

In some embodiments of the methods provided herein, a tablet comprising: about 35 w/w % to about 45 w/w % of the compound of Formula Ia, or a pharmaceutically acceptable salt thereof,
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:

about 35 w/w % to about 45 w/w % of the compound of Formula Ia, or a pharmaceutically acceptable salt thereof;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ia, or a pharmaceutically acceptable salt thereof;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ia, or a pharmaceutically acceptable salt thereof;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 40 w/w % of the compound of Formula Ia, or a pharmaceutically acceptable salt thereof;
about 27.25 w/w % of mannitol;
about 27.25 w/w % of microcrystalline cellulose;
about 4 w/w % of crospovidone; and
about 1.5 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ia;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ia;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ia;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ia;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 40 w/w % of the compound of Formula Ia;
about 27.25 w/w % of mannitol;
about 27.25 w/w % of microcrystalline cellulose;
about 4 w/w % of crospovidone; and
about 1.5 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib, or a pharmaceutically acceptable salt thereof;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib, or a pharmaceutically acceptable salt thereof;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib, or a pharmaceutically acceptable salt thereof;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib, or a pharmaceutically acceptable salt thereof;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 40 w/w % of the compound of Formula Ib, or a pharmaceutically acceptable salt thereof;
about 27.25 w/w % of mannitol;
about 27.25 w/w % of microcrystalline cellulose;
about 4 w/w % of crospovidone; and
about 1.5 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 40 w/w % of the compound of Formula Ib;
about 27.25 w/w % of mannitol;
about 27.25 w/w % of microcrystalline cellulose;
about 4 w/w % of crospovidone; and
about 1.5 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the crystalline form of the compound of Formula Ib;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the crystalline form of the compound of Formula Ib;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the crystalline form of the compound of Formula Ib;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the crystalline form of the compound of Formula Ib;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 40 w/w % of the crystalline form of the compound of Formula Ib;
about 27.25 w/w % of mannitol;
about 27.25 w/w % of microcrystalline cellulose;
about 4 w/w % of crospovidone; and
about 1.5 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the crystalline form of the compound of Formula Ib, crystalline Form I;
about 27.0 w/w % to about 28 w/w % of mannitol;
about 27.0 w/w % to about 28 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib, crystalline Form I;
about 27.1 w/w % to about 27.6 w/w % of mannitol;
about 27.1 w/w % to about 27.6 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib, crystalline Form I;
about 27.2 w/w % to about 27.3 w/w % of mannitol;
about 27.2 w/w % to about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 35 w/w % to about 45 w/w % of the compound of Formula Ib, crystalline Form I;
about 27.3 w/w % of mannitol;
about 27.3 w/w % of microcrystalline cellulose;
about 2 w/w % to about 6 w/w % of crospovidone; and
about 1 w/w % to about 3 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 40 w/w % of the compound of Formula Ib, crystalline Form I;
about 27.25 w/w % of mannitol;
about 27.25 w/w % of microcrystalline cellulose;
about 4 w/w % of crospovidone; and
about 1.5 w/w % of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising about 175 mg to about 225 mg of the compound of Formula Ia, or a pharmaceutically acceptable salt thereof, and one or more pharmaceutically acceptable excipients, is administered orally to a patient in need thereof, once every seven days.

In some embodiments of the methods provided herein, a tablet comprising about 200 mg of the compound of Formula Ia, or a pharmaceutically acceptable salt thereof, and one or more pharmaceutically acceptable excipients, is administered orally to a patient in need thereof, once every seven days.

In some embodiments of the methods provided herein, a tablet comprising about 175 mg to about 225 mg of the compound of Formula Ia, and one or more pharmaceutically acceptable excipients, is administered orally to a patient in need thereof, once every seven days.

In some embodiments of the methods provided herein, a tablet comprising about 200 mg of the compound of Formula Ia, and one or more pharmaceutically acceptable excipients, is administered orally to a patient in need thereof, once every seven days.

In some embodiments of the methods provided herein, a tablet comprising about 175 mg to about 225 mg of the compound of Formula Ib, or a pharmaceutically acceptable salt thereof, and one or more pharmaceutically acceptable excipients, is administered orally to a patient in need thereof, once every seven days.

In some embodiments of the methods provided herein, a tablet comprising about 200 mg of the compound of Formula Ib, or a pharmaceutically acceptable salt thereof, and one or more pharmaceutically acceptable excipients, is administered orally to a patient in need thereof, once every seven days.

In some embodiments of the methods provided herein, a tablet comprising about 175 mg to about 225 mg of the compound of Formula Ib, and one or more pharmaceutically acceptable excipients, is administered orally to a patient in need thereof, once every seven days.

In some embodiments of the methods provided herein, a tablet comprising about 200 mg of the compound of Formula Ib, and one or more pharmaceutically acceptable excipients, is administered orally to a patient in need thereof, once every seven days.

In some embodiments of the methods provided herein, a tablet comprising about 175 mg to about 225 mg of a crystalline form of the compound of Formula Ib, and one or more pharmaceutically acceptable excipients, is administered orally to a patient in need thereof, once every seven days.

In some embodiments of the methods provided herein, a tablet comprising about 200 mg of a crystalline form of the compound of Formula Ib, and one or more pharmaceutically acceptable excipients, is administered orally to a patient in need thereof, once every seven days.

In some embodiments of the methods provided herein, a tablet comprising about 175 mg to about 225 mg a compound of Formula Ib, crystalline Form I, and one or more pharmaceutically acceptable excipients, is administered orally to a patient in need thereof, once every seven days.

In some embodiments of the methods provided herein, a tablet comprising about 200 mg of a compound of Formula Ib, crystalline Form I, and one or more pharmaceutically acceptable excipients, is administered orally to a patient in need thereof, once every seven days.

In some embodiments of the methods provided herein, a tablet comprising:
about 175 mg to about 225 mg of the compound of Formula Ia, or a pharmaceutically acceptable salt thereof;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising: about 200 mg of the compound of Formula Ia, or a pharmaceutically acceptable salt thereof;
about 135 mg to about 137 mg of mannitol;
about 135 mg to about 137 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ia, or a pharmaceutically acceptable salt thereof;
about 136.2 mg of mannitol;
about 136.2 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ia, or a pharmaceutically acceptable salt thereof;
about 136.3 mg of mannitol;
about 136.3 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ia, or a pharmaceutically acceptable salt thereof;
about 136.25 mg of mannitol;
about 136.25 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 175 mg to about 225 mg of the compound of Formula Ia;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ia;
about 135 mg to about 137 mg of mannitol;
about 135 mg to about 137 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ia;
about 136.2 mg of mannitol;
about 136.2 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ia;
about 136.3 mg of mannitol;
about 136.3 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ia;
about 136.25 mg of mannitol;
about 136.25 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 175 mg to about 225 mg of the compound of Formula Ib, or a pharmaceutically acceptable salt thereof;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ib, or a pharmaceutically acceptable salt thereof;
about 135 mg to about 137 mg of mannitol;
about 135 mg to about 137 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ib, or a pharmaceutically acceptable salt thereof;
about 136.2 mg of mannitol;
about 136.2 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ib, or a pharmaceutically acceptable salt thereof;
about 136.3 mg of mannitol;
about 136.3 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ib, or a pharmaceutically acceptable salt thereof;
about 136.25 mg of mannitol;
about 136.25 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 175 mg to about 225 mg of the compound of Formula Ib;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ib;
about 135 mg to about 137 mg of mannitol;
about 135 mg to about 137 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ib;
about 136.2 mg of mannitol;
about 136.2 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ib;
about 136.3 mg of mannitol;
about 136.3 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of the compound of Formula Ib;
about 136.25 mg of mannitol;
about 136.25 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 175 mg to about 225 mg of the crystalline form of the compound of Formula Ib;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of a crystalline form of the compound of Formula Ib;
about 135 mg to about 137 mg of mannitol;
about 135 mg to about 137 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of a crystalline form of the compound of Formula Ib;
about 136.2 mg of mannitol;
about 136.2 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of a crystalline form of the compound of Formula Ib;
about 136.3 mg of mannitol;
about 136.3 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of a crystalline form of the compound of Formula Ib;
about 136.25 mg of mannitol;
about 136.25 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 175 mg to about 225 mg of a compound of Formula Ib, crystalline Form I;
about 135 mg to about 140 mg of mannitol;
about 135 mg to about 140 mg of microcrystalline cellulose;
about 15 mg to about 25 mg of crospovidone; and
about 5 mg to about 10 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of a compound of Formula Ib, crystalline Form I;
about 135 mg to about 137 mg of mannitol;
about 135 mg to about 137 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of a compound of Formula Ib, crystalline Form I;
about 136.2 mg of mannitol;
about 136.2 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of a compound of Formula Ib, crystalline Form I;
about 136.3 mg of mannitol;
about 136.3 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 200 mg of a compound of Formula Ib, crystalline Form I;
about 136.25 mg of mannitol;
about 136.25 mg of microcrystalline cellulose;
about 20 mg of crospovidone; and
about 7.5 mg of magnesium stearate;
is administered orally to a patient in need thereof, once every seven days. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the compound of Formula Ia, or a pharmaceutically acceptable salt thereof;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the compound of Formula Ia, or a pharmaceutically acceptable salt thereof;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the compound of Formula Ia, or a pharmaceutically acceptable salt thereof;
about 7.8 w/w % of mannitol;
about 7.8 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the compound of Formula Ia, or a pharmaceutically acceptable salt thereof;
about 7.75 w/w % of mannitol;
about 7.75 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the compound of Formula Ia;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the compound of Formula Ia;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the compound of Formula Ia;
about 7.8 w/w % of mannitol;
about 7.8 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the compound of Formula Ia;
about 7.75 w/w % of mannitol;
about 7.75 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the compound of Formula Ib, or a pharmaceutically acceptable salt thereof,
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the compound of Formula Ib, or a pharmaceutically acceptable salt thereof;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the compound of Formula Ib, or a pharmaceutically acceptable salt thereof;
about 7.8 w/w % of mannitol;
about 7.8 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the compound of Formula Ib, or a pharmaceutically acceptable salt thereof;
about 7.75 w/w % of mannitol;
about 7.75 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the compound of Formula Ib;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the compound of Formula Ib;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the compound of Formula Ib;
about 7.8 w/w % of mannitol;
about 7.8 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the compound of Formula Ib;
about 7.75 w/w % of mannitol;
about 7.75 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the crystalline form of the compound of Formula Ib;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the crystalline form of the compound of Formula Ib;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the crystalline form of the compound of Formula Ib;
about 7.8 w/w % of mannitol;
about 7.8 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the crystalline form of the compound of Formula Ib;
about 7.75 w/w % of mannitol;
about 7.75 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the compound of Formula Ib, crystalline Form I;
about 5 w/w % to about 10 w/w % of mannitol;
about 5 w/w % to about 10 w/w % of microcrystalline cellulose;
about 5 w/w % to about 10 w/w % of crospovidone; and
about 1 w/w % to about 5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 70 w/w % to about 80 w/w % of the compound of Formula Ib, crystalline Form I;
about 6 w/w % to about 9 w/w % of mannitol;
about 6 w/w % to about 9 w/w % of microcrystalline cellulose;
about 6 w/w % to about 9 w/w % of crospovidone; and
about 1 w/w % to about 3 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the compound of Formula Ib, crystalline Form I;
about 7.8 w/w % of mannitol;
about 7.8 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 75 w/w % of the compound of Formula Ib, crystalline Form I;
about 7.75 w/w % of mannitol;
about 7.75 w/w % of microcrystalline cellulose;
about 8 w/w % of crospovidone; and
about 1.5 w/w % of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1000 mg to about 1500 mg of the compound of Formula Ia, or a pharmaceutically acceptable salt thereof;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1100 mg to about 1300 mg of the compound of Formula Ia, or a pharmaceutically acceptable salt thereof;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1200 mg of the compound of Formula Ia, or a pharmaceutically acceptable salt thereof;
about 124 mg of mannitol;
about 124 mg of microcrystalline cellulose;
about 128 mg of crospovidone;
about 24 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1000 mg to about 1500 mg of the compound of Formula Ia;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1100 mg to about 1300 mg of the compound of Formula Ia;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1200 mg of the compound of Formula Ia;
about 124 mg of mannitol;
about 124 mg of microcrystalline cellulose;
about 128 mg of crospovidone;
about 24 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1000 mg to about 1500 mg of the compound of Formula Ib, or a pharmaceutically acceptable salt thereof;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1100 mg to about 1300 mg of the compound of Formula Ib, or a pharmaceutically acceptable salt thereof;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1200 mg of the compound of Formula Ib, or a pharmaceutically acceptable salt thereof;
about 124 mg of mannitol;
about 124 mg of microcrystalline cellulose;
about 128 mg of crospovidone;
about 24 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1000 mg to about 1500 mg of the compound of Formula Ib;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1100 mg to about 1300 mg of the compound of Formula Ib;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1200 mg of the compound of Formula Ib;
about 124 mg of mannitol;
about 124 mg of microcrystalline cellulose;
about 128 mg of crospovidone;
about 24 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1000 mg to about 1500 mg of the crystalline form of the compound of Formula Ib;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1100 mg to about 1300 mg of the crystalline form of the compound of Formula Ib;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1200 mg of the crystalline form of the compound of Formula Ib;
about 124 mg of mannitol;
about 124 mg of microcrystalline cellulose;
about 128 mg of crospovidone;
about 24 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1000 mg to about 1500 mg of the compound of Formula Ib, crystalline Form I;
about 120 mg to about 130 mg of mannitol;
about 120 mg to about 130 mg of microcrystalline cellulose;
about 100 mg to about 150 mg of crospovidone;
about 20 mg to about 30 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1100 mg to about 1300 mg of the compound of Formula Ib, crystalline Form I;
about 123 mg to about 125 mg of mannitol;
about 123 mg to about 125 mg of microcrystalline cellulose;
about 127 mg to about 129 mg of crospovidone;
about 22 mg to about 28 mg of magnesium stearate;
is administered orally to a patient in need thereof, once every month. In some embodiments, the above tablet consists of the specified ingredients. In some embodiments, the above tablet consists essentially of the specified ingredients.

In some embodiments of the methods provided herein, a tablet comprising:
about 1200 mg of the compound of Formula Ib, crystalline Form I;
about 124 mg of mannitol;
about 124 mg of microcrystalline cellulose;
about 128 mg of crospovidone;
about 24 mg of magnesium stearate;
is administered orally to a patient in need thereof, once every month. In some embodiments, the above tablet consists of the specified ingredients. In some embodiments, the above tablet consists essentially of the specified ingredients.

### EXAMPLES

The disclosure will be described in greater detail by way of specific examples. The following examples are offered for illustrative purposes, and are not intended to limit the disclosure in any manner. Those of skill in the art will readily recognize a variety of noncritical parameters which can be changed or modified to yield essentially the same results.

### Example 1. Tablet Formulations

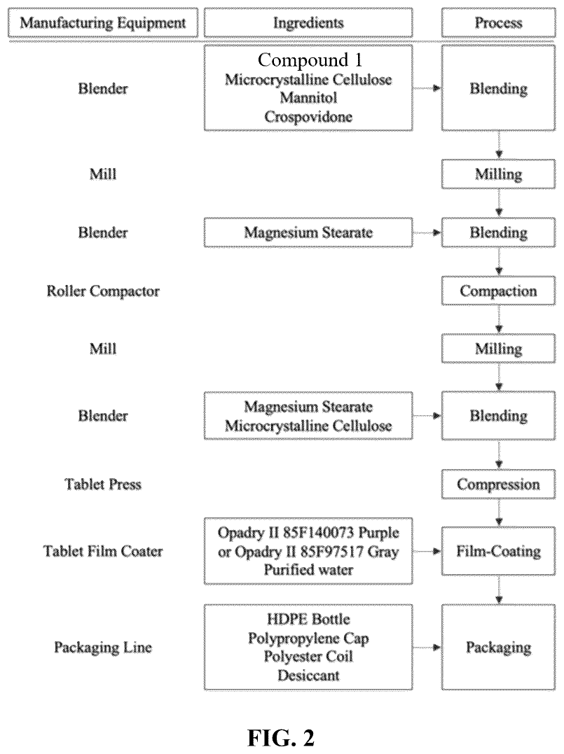

The following tablet formulations of Table 1 were prepared according to the manufacturing process flow diagram shown in FIG. 2. The tablets were prepared by dry granulation process, where the intragranular mixture was blended, milled, blended, compacted via rolled compactor (RC), blended, and then formed into tablets and coated. Film coatings represent a theoretical target weight gain of 4% or 3% as indicated in Table 1. Purified water was used to prepare the film-coating suspension and removed during the film-coating process.

TABLE 1
Tablet Formulations

| | Composition | | | | | |
|---|---|---|---|---|---|---|
| | | mg/tablet | | | | |
| | % | 100 | 200 | 300 | 400 | |
| Ingredient | w/w | mg | mg | mg | mg | Function |

Tablet Core

| | | | | | | |
|---|---|---|---|---|---|---|
| Compound 1 | 40.00 | 100.00 | 200.00 | 300.00 | 400.00 | Active |
| | | | | | | Ingredient |
| Mannitol | 27.00 | 67.50 | 135.00 | 202.50 | 270.00 | Diluent |
| Micro- | 27.50 | 68.75 | 137.50 | 206.25 | 275.00 | Diluent |
| crystalline | | | | | | |
| Cellulose | | | | | | |
| Crospovidone | 4.00 | 10.00 | 20.00 | 30.00 | 40.00 | Disintegrant |
| Magnesium | 1.50 | 3.75 | 7.50 | 11.25 | 15.00 | Lubricant |
| Stearate | | | | | | |
| Tablet Core | 100.0 | 250.0 | 500.0 | 750.0 | 1000.0 | — |
| Total | | | | | | |

Film Coat

| | | | | | |
|---|---|---|---|---|---|
| Opadry II | 4.0 | 10.0 | | 40.0 | Film-coat |
| Purple | | | | | |
| 85F140073 | | | | | |
| Opadry | 3.0 | | 22.5 | | Film-coat |
| II Gray | | | | | |
| 85F97517 | | | | | |

### Example 2. In Vivo Dog Studies

A tablet, prepared from the crystalline free acid of Compound 1, crystalline Form I, was prepared in a composition shown in Table 2 below. The tablet was administered orally at 55 mg fixed dose to 3 beagle dogs and 100 mg fixed dose to 6 beagle dogs and the pharmacokinetic (PK) profile was determined. A 55 mg-fixed dose of Compound 1 is equivalent to a 40 mg-fixed dose of lenacapavir (LEN). Non-Precipitating Solution: 5% Ethanol, 20% PG, 75% Water, pH 7.33, n=3. Dry Granulated Tablets: 40.4% Compound 1, 27.3% microcrystalline cellulose, 27.3% mannitol, 4.0 n crospovidone, 1% magnesium Stearate, n=3 for 55 mg dose and n=6 for 100 mg dose. LEN SDD (spray dried dispersion) Tablets: 200% SDD API, 5.34% copovidone, 8% croscarmellose sodium, 32.16% lactose, 1% magnesium stearate, 32.16% microcrystalline cellulose, 1.34% poloxamer 407, n=6.

TABLE 2

| | | | API | | | | |
|---|---|---|---|---|---|---|---|
| | | | Particle | Dose | Mean | Mean | |
| | | Pre- | Size | (mg- | AUC₍₀₋ₗₐₛₜ₎ ± | Cₘₐₓ ± | |
| API | Formulation | treatment | (d90 μm) | fixed) | SD (nM*h) | SD (nM) | % F |
| Compound | Non- | Fasted, | — | 55 | 12,017 ± 2,694 | 861 ± 166 | 16.4 ± 3.7 |
| 1 | Precipitating | Pentagastrin | | | 13,040 ± 1,761 | 884 ± 244 | 18.1 ± 0.4 |
| | Solution | | | | | | |
| | Dry | Fasted, | 85 | 100 | 16,949 ± 6039 | 1,403 ± 604 | 14.5 ± 5.9 |
| | Granulated | Pentagastrin | | | | | |
| | Tablet | | | | | | |
| | Dry | Fasted, | 85 | 55 | 12,386 ± 6,948 | 817 ± 523 | 16.3 ± 8.3 |
| | Granulated | Pentagastrin | | | | | |
| | Tablet | | | | | | |
| LEN | SDD | Fasted, | — | 40 | 2,822 ± 511 | 121 ± 49 | 4.6 ± 1 |
| | Tablets | Pentagastrin | | | | | |

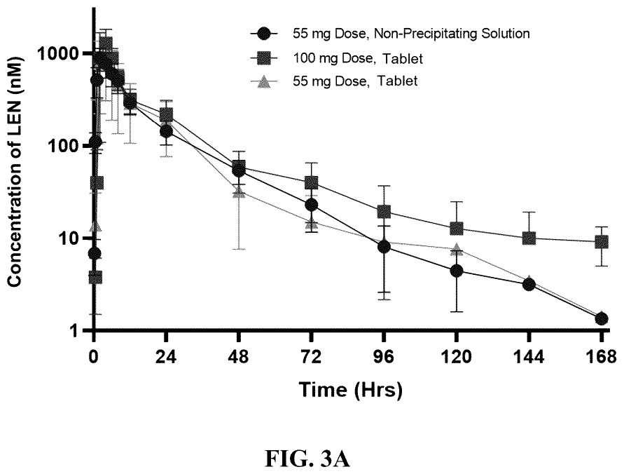

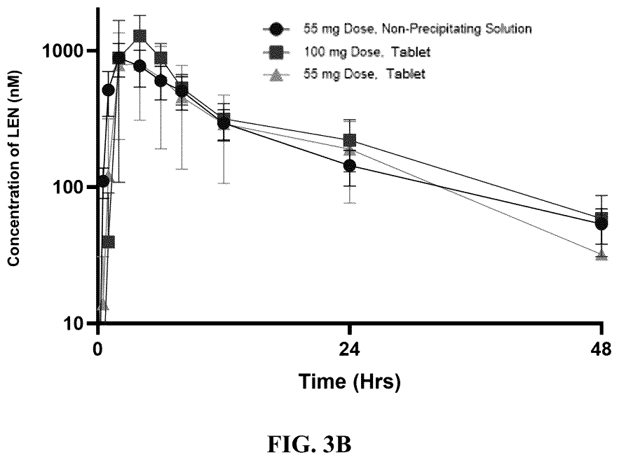

FIGS. 3A-3B show plots of the plasma concentration of lenacapavir (LEN) as a function of time. Higher exposure of LEN was achieved when dosed as a crystalline tablet of Compound 1, crystalline Form I, than when dosed as a LEN SDD Tablet.

### Example 3. Preparation of Compound 1, Crystalline Form I

### Preparation of Compound 1, Material A

A solution containing 0.1 g of Compound 1 in 0.2 mL of DCM was mixed with 1 eq. of sulfonic acid, charged with 0.05 mL of toluene, and stirred with a magnetic stir bar at about 22° C. During the 16-hour stirring period, a slurry formed. The slurry was centrifuged and XRPD analysis of the wet cake showed it was crystalline. After drying, the solid lost crystallinity and became Compound 1, Material A.

Compound 1, Material A was also obtained as follows. About 1 g of Compound 1, amorphous material was stirred in 1 mL of EtOAc to form a solution. It was seeded with Compound 1, crystalline Material A (prepared as described above), sonicated, and crystallized as a thick slurry within an hour. After drying, the solid lost crystallinity and became Compound 1, Material A.

### Preparation of Compound 1, Crystalline Form I

About 50 mg of Compound 1, Material A was stirred in 0.1 mL of MeCN at about 22° C. over a period of 20 hours. XRPD pattern of the wet cake, designated as MeCN solvate. After drying at 50° C. under vacuum, it became Compound 1, crystalline Form I.

Compound 1, crystalline Form I was also obtained after drying at 50 to 75° C. from other solvates (as precursors) that had been obtained by stirring Compound 1 in the corresponding solvents (preferably with the seeds of Compound 1, crystalline Form I) with an appropriate antisolvent such as diisopropyl ether, n-butyl ether, or heptanes, if necessary.

Compound 1, crystalline Form I was also obtained from amorphous Compound 1. 5 g of amorphous Compound 1 was stirred in 5 mL MeCN in a vial at about 22° C. While most of the solid dissolved, some sticky substance remained at the bottom of the vial. The sample was sonicated for a few minutes, and a thick slurry was obtained. The slurry was diluted with 5 mL of MeCN and 5 mL of DIPE. After stirring for 30 min at 22° C., the slurry was then cooled to 3° C. over 30 min. The slurry was further diluted with 15 mL of DIPE. After stirring for an additional 30 min, 10 mL heptanes were added. The mixture became sticky after awhile at 3° C. The mixture was heated back to 22° C. and was sonicated to become a slurry. The slurry was stirred for two days at 22° C. and was filtered, washed with 10 mL of DIPE, and then 10 mL of heptanes. The wet cake was dried in the vacuum oven at 50° C. overnight to provide Compound 1, crystalline Form I, which was confirmed by XRPD analysis.

A representative XRPD pattern of Compound 1, crystalline Form I (after drying the MeCN solvate at 50° C. under vacuum) is shown in FIG. 4 and is characterized by sharp reflections, indicating crystallinity. An XRPD peak listing is provided in Table 1-A.

TABLE 3

| 2-Theta (°) | Relative Intensity (%) |
|---|---|
| 6.2 | 100 |
| 6.6 | 14 |
| 8.7 | 26 |
| 10.5 | 18 |
| 11.0 | 4 |
| 12.4 | 23 |
| 12.6 | 17 |
| 13.2 | 8 |
| 13.8 | 57 |
| 16.1 | 20 |
| 16.5 | 12 |
| 17.0 | 8 |
| 17.5 | 14 |
| 17.7 | 16 |
| 18.0 | 7 |
| 18.6 | 15 |
| 19.8 | 14 |
| 20.7 | 19 |
| 21.5 | 11 |
| 22.3 | 14 |
| 23.1 | 28 |
| 23.4 | 11 |
| 24.2 | 8 |
| 25.7 | 36 |
| 26.3 | 20 |
| 29.3 | 5 |
| 30.2 | 10 |

The DSC thermogram of Compound 1, crystalline Form I indicated an endotherm event with onset at about 202° C., attributed to melting, and is shown in FIG. 5. The TGA thermogram, shown in FIG. 6, showed a weight loss of about 0.04% (from 25 to 150° C.), indicating that the solid is not solvated. DVS analysis indicated that the form is hygroscopic with about 4% water uptake from 0 to 90% RH at 25° C., as shown in FIG. 7.

### Example 4. Safety and Efficacy of Twice-Yearly, Subcutaneous Lenacapavir for Pre-Exposure Prophylaxis (PrEP)

PURPOSE 1, a Phase 3, double-blind, randomized study, is evaluating the safety and efficacy of twice-yearly, subcutaneous lenacapavir for pre-exposure prophylaxis (PrEP) and once-daily oral Descovy® (emtricitabine 200 mg and tenofovir alafenamide 25 mg; F/TAF) in more than 5,300 cisgender women and adolescent girls aged 16-25 across 25 sites in South Africa and three sites in Uganda. The drugs are being tested in parallel, with one group receiving twice-yearly lenacapavir and one group taking once-daily oral Descovy. Additionally, a third group was assigned once-daily oral Truvada. Study participants were randomized in a 2:2:1 ratio to lenacapavir, Descovy and Truvada, respectively. Because effective PrEP options already exist, there is broad consensus in the PrEP field that a placebo group would be unethical; thus, the trial used background HIV incidence (bHIV) as the primary comparator and Truvada as a secondary comparator.

There were 0 incident cases of HIV infection among women in the lenacapavir group (incidence 0.00 per 100 person-years). The results demonstrated superiority of twice-yearly lenacapavir over bHIV (primary endpoint, incidence 2.41 per 100 person-years) and superiority of twice-yearly lenacapavir over once-daily Truvada (secondary endpoint), with p<0.0001 for both endpoints. In the trial, lenacapavir was generally well-tolerated and no significant or new safety concerns were identified. HIV incidence in the Descovy group was numerically similar to that in the Truvada group and was not statistically superior to bHIV.

### Example 5. Pharmacokinetic and Pharmacodynamic Evaluation of Compound 1

### Summary of Pharmacokinetic Results

Following oral administration, Compound 1 was converted to LEN and other metabolites. Compound 1 levels were undetectable in plasma for the majority of the samples in the study. Transient plasma levels of Compound 1 in limited participants were not sufficient to generate Compound 1 PK parameters.

### Single Ascending Dose

Following single doses of Compound 1, LEN exposures increased less than dose-proportionally over the dose range of 200 mg to 2400 mg. A 12-fold increase in Compound 1 dose resulted in a 3.8-fold increase in AUC_{inf} and a 10.5-fold increase in Cₘₐₓ. The median Tₘₐₓ of LEN was 7.02 to 48.0 hours, and the median t_{1/2} of LEN was between 241 and 273 hours (i.e., 10.0 to 11.4 days).

### Multiple Ascending Weekly Dosing

Following multiple doses of Compound 1 200 mg and 400 mg once weekly for 6 weeks, steady-state Cₘₐₓ was 6.15- and 6.68-fold higher, C_{trough} was 3.49- and 2.86-fold higher, and AUCₜₐᵤ was 4.42- and 3.72-fold higher, respectively, indicating accumulation at Week 6.

### Effect of Food

Effect of low-fat meal on 1800 mg dose: A positive food effect on LEN exposures (8.87-fold higher Cₘₐₓ and 3.02-fold higher AUC_{inf}) was observed with a low-fat meal at a dose of Compound 1 1800 mg.

Effect of high-fat meal on 400 mg dose: A positive food effect on LEN exposures (3.27-fold higher Cₘₐₓ and 2.69-fold higher AUC₀₋₁₆₈ₕ) was observed with a high-fat meal at a clinically relevant dose of Compound 1 400 mg.

### Urine Pharmacokinetics

Urine samples for all single ascending dose (SAD) and multiple ascending dose (MAD) cohorts at all time points were below the limit of quantification (BLQ) (<5 ng/mL) for Compound 1, which indicates negligible renal elimination of unchanged Compound 1. The fraction excreted in urine was <1% for LEN across SAD and MAD doses, indicating negligible or minimal renal elimination.

### Example 6. Plasma Pharmacokinetics of Compound 1 and Metabolites

Following oral administration, Compound 1 was converted to LEN and other metabolites. In SAD cohorts, Compound 1 levels were detectable in plasma up to 24 hours postdose in 6% participants, and in MAD cohorts, Compound 1 levels were detectable in plasma up to 12 hours postdose in 11% participants. These transient plasma levels of Compound 1 in limited participants were not sufficient to generate Compound 1 PK parameters.

### Single-Dose Pharmacokinetics

Participants in Cohorts 1, 2, 4, and 5 (N=8 per cohort) received a single oral dose of Compound 1 in the fasted state (dose range: 200 mg to 2400 mg). In SAD cohorts, Compound 1 was undetected in the majority of the participants (94%). It was detectable in plasma in 2 of 32 (6%) participants from 600 mg (up to 4 hours postdose for 1 participant) and 2400 mg (up to 24 hours postdose for 1 participant) doses with no detectable levels at 200 mg and 1800 mg doses. These transient plasma levels of Compound 1 in limited participants were not sufficient to generate Compound 1 PK parameters.

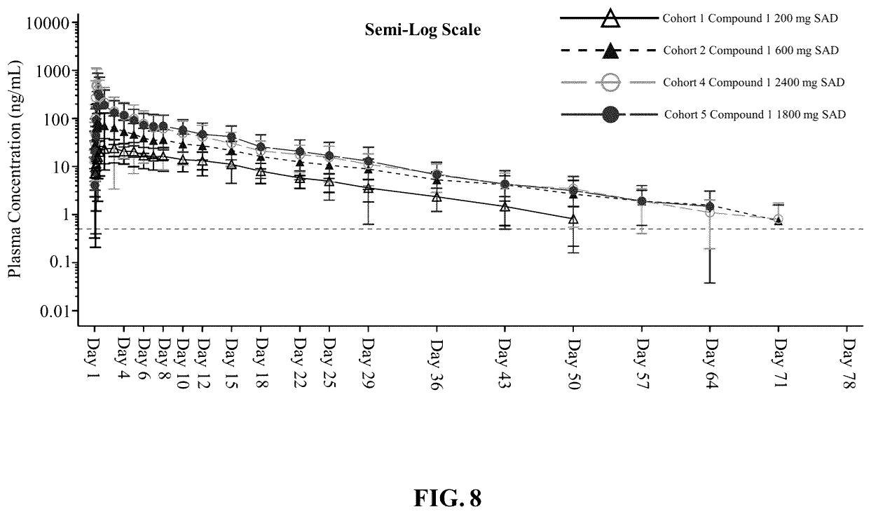

The LEN concentration-time profiles after single-dose administration of Compound 1 are presented in FIG. 8. In SAD cohorts, LEN exposures increased less than dose-proportionally over the dose range of 200 mg to 2400 mg (Table 4). A 12-fold increase in Compound 1 dose from 200 mg to 2400 mg resulted in a 3.8-fold increase in AUC_{inf} and a 10.5-fold increase in Cₘₐₓ. Lenacapavir exposure (AUC) following single-dose administration of Compound 1 1800 mg and 2400 mg was similar, indicating saturation in absorption. The median Tₘₐₓ of LEN was 7.02 to 48.0 hours and the median t_{1/2} of LEN was between 241 and 273 hours (i.e., 10.0 to 11.4 days).

TABLE 4
Plasma PK Parameters of LEN Following Single Doses
of Compound 1 (SAD Cohorts, PK Analysis Sets)

| | Cohort 1 | | Cohort 2 | | Cohort 4 | | Cohort 5 | |
|---|---|---|---|---|---|---|---|---|
| Analyte/ PK | 200 mg Fasted | | 600 mg Fasted | | 2400 mg Fasted | | 1800 mg Fasted | |
| Parameter | (N = 8) | | (N = 8) | | (N = 8) | | (N = 8) | |
| Cₘₐₓ (ng/mL) | 22.1 | (60.3) | 61.7 | (111) | 233 | (285) | 168 | (154) |
| C_{168 h} | 14.5 | (54.8) | 31.1 | (63.5) | 48.3 | (77.8) | 59.8 | (60.7) |
| (ng/mL) | | | | | | | | |
| AUC_{0-168 h} | 2870 | (58.3) | 7050 | (72.7) | 14,700 | (117) | 15,100 | (77.8) |
| (h · ng/mL) | | | | | | | | |
| AUCₗₐₛₜ | 7860 | (54.9) | 17,800 | (63.7) | 30,200 | (85.7) | 33,200 | (62.6) |
| (h · ng/mL) | | | | | | | | |
| AUC_{inf} | 8120 | (53.3) | 18,500 | (61.5) | 30,600 | (85.2) | 33,500 | (62.4) |
| (h · ng/mL) | | | | | | | | |
| Tₘₐₓ (h) | 48.0 | (24.0, 48.0) | 24.0 | (10.0, 36.3) | 7.02 | (4.03, 16.1) | 12.0 | (8.00, 18.0) |
| Tₗₐₛₜ (h) | 1340 | (1090, 1600) | 1680 | (1510, 1850) | 1670 | (1510, 1760) | 1680 | (1340, 1850) |
| t_{1/2} (h) | 258 | (218, 294) | 273 | (250, 285) | 260 | (233, 278) | 241 | (209, 267) |

% GCV = percentage geometric coefficient of variation;
LEN = lenacapavir;
PK = pharmacokinetic(s);
Q1 = first quartile;
Q3 = third quartile;
SAD = single ascending dose

### Dose Proportionality Analysis of Single-Dose Plasma Pharmacokinetics

The statistical comparisons of LEN dose-normalized PK parameter estimates for dose proportionality are provided in Table 5. The results of the ANOVA and power model analyses are in agreement, indicating less than dose-proportional increases in LEN Cₘₐₓ, AUCₗₐₛₜ, and AUC_{inf} between Compound 1 doses 200 mg to 2400 mg. For doses between 200 mg and 600 mg, the LEN Cₘₐₓ increase was close to dose proportional, and AUC was slightly less than dose proportional, however the 90% CI ranged from 46.6% to 180% (Table 5).

TABLE 5
Statistical Comparisons of Dose-Normalized LEN PK Parameters
for Dose Proportionality (SAD Cohorts, PK Analysis Set)

| | | | | | Statistical |
|---|---|---|---|---|---|
| | Treatment | | | | Comparison |
| | Test | | Reference | | % GLSM Ratio |
| PK Parameter | n | GLSM | n | GLSM | (90% CI) |

Compound 1 600 mg (Test) vs Compound 1 200 mg (Reference)

| | | | | | | |
|---|---|---|---|---|---|---|
| Cₘₐₓ/D (ng/mL) | 8 | 20.6 | 8 | 22.1 | 93.1 | (48.3, 180) |
| AUCₗₐₛₜ/D | 8 | 5950 | 8 | 7860 | 75.6 | (46.6, 123) |
| (h · ng/mL) | | | | | | |
| AUC_{inf}/D (h · ng/mL) | 8 | 6170 | 8 | 8120 | 76.0 | (47.5, 122) |

Compound 1 2400 mg (Test) vs Compound 1 200 mg (Reference)

| | | | | | | |
|---|---|---|---|---|---|---|
| Cₘₐₓ/D (ng/mL) | 8 | 19.4 | 8 | 22.1 | 87.8 | (32.7, 236) |
| AUCₗₐₛₜ/D | 8 | 2520 | 8 | 7860 | 32.0 | (18.3, 56.2) |
| (h · ng/mL) | | | | | | |
| AUC_{inf}/D (h · ng/mL) | 8 | 2550 | 8 | 8120 | 31.4 | (18.0, 54.7) |

ANOVA = analysis of variance;
CI = confidence interval;
GLSM = geometric least-squares mean;
LEN = lenacapavir;
PK = pharmacokinetic(s);
SAD = single ascending dose
The ANOVA model includes dose as a fixed effect. The PK parameters are dose normalized to 200 mg.

### Example 7. Multiple-Dose Pharmacokinetics

Participants in Cohorts 6 and 7 (N=9 per cohort) received multiple oral doses of Compound 1 in the fasted state (dose range: 200 mg and 400 mg). In MAD cohorts, Compound 1 levels were detectable in plasma in 2 of 18 (11%) participants (up to 2 or 12 hours postdose in 2 participants from Cohort 6; none in Cohort 7). These transient plasma levels of Compound 1 in limited participants were not sufficient to generate Compound 1 PK parameters.

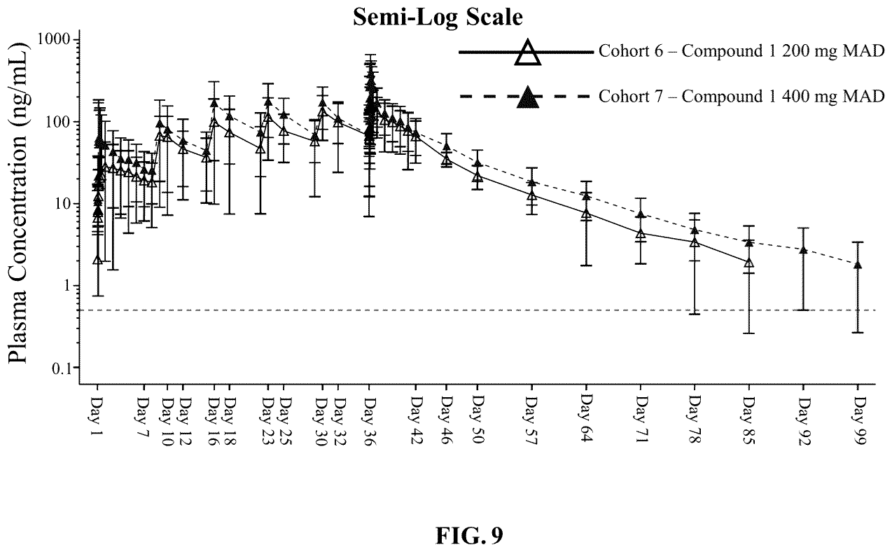

The LEN concentration-time profiles after once-weekly administration of Compound 1 200 mg (Cohort 6) and 400 mg (Cohort 7) for 6 weeks are shown in FIG. 9. Following 200 mg once weekly, Week 6 Cₘₐₓ, C_{trough}, and AUCₜₐᵤ were 6.15-fold, 3.49-fold, and 4.42-fold higher, respectively, compared with Week 1 estimates (Table 6). Similarly, following 400 mg once weekly, Week 6 Cₘₐₓ, C_{trough}, and AUCₜₐᵤ for LEN were 6.68-fold, 2.86-fold, and 3.72-fold higher, respectively, compared with Week 1 estimates. Steady-state for LEN was achieved by Week 5 following 200 mg and 400 mg once-weekly administration.

TABLE 6
Plasma PK Parameters of LEN Following Multiple Once-Weekly
Doses of Compound 1 (MAD Cohorts, PK Analysis Sets)

| | Cohort 6 | | | | Cohort 7 | | | |
|---|---|---|---|---|---|---|---|---|
| | 200 mg QW Fasted | | | | 400 mg QW Fasted | | | |
| Analyte/ PK | Week 1 | | Week 6 | | Week 1 | | Week 6 | |
| Parameter | (N = 9) | | (N = 8) | | (N = 9) | | (N = 9) | |
| Cₘₐₓ (ng/mL) | 24.7 | (68.8) | 153 | (76.7) | 44.7 | (141) | 298 | (124) |
| C_{168 h} (ng/mL) | 14.5 | (70.5)^{a} | 51.7 | (43.2) | 21.1 | (63.3) | 60.4 | (53.7) |
| AUCₜₐᵤ | 3150 | (72.8)^{a} | 14,100 | (57.8) | 4810 | (85.1) | 17,900 | (65.2) |

| (h · ng/mL) | | | | | | |
|---|---|---|---|---|---|---|
| Tₘₐₓ (h) | 72.0 (24.0, | 6.02 (4.02, | | 12.0 (4.00, | 4.00 (3.00, | |
| | 72.0) | 16.0) | | 24.1) | 11.8) | |
| t_{1/2} (h) | — | 246 | (200, 276) | — | 293 | (280, 316) |

| | | | | |
|---|---|---|---|---|
| Accumulation | — | 6.15 (3.74, | — | 6.68 (2.91, |
| ratio of Cₘₐₓ | | 10.1) | | 15.3) |
| Accumulation | — | 3.49 (2.33, | — | 2.86 (1.91, |
| ratio of C_{168 h} | | 5.24) | | 4.30) |
| Accumulation | — | 4.42 (2.81, | — | 3.72 (2.21, |
| ratio of AUCₜₐᵤ | | 6.94) | | 6.25) |

CI = confidence interval;
% GCV = percentage geometric coefficient of variation;
GLSM = geometric least-squares mean;
LEN = lenacapavir;
MAD = multiple ascending dose;
PK = pharmacokinetic(s);
Q1 = first quartile;
Q3 = third quartile;
QW = once weekly
^{a}N = 8.
Cₜₐᵤ = C_{168 h} = C_{trough}.
AUCₜₐᵤ = AUC_{0-168 h}.
All data are presented as geometric mean (% GCV) except time parameters, which are presented as median (Q1, Q3) and accumulation ratio, which is shown as GLSM ratio (90% CI).

### Dose Proportionality Analysis of Multiple Dose Plasma Pharmacokinetics

The statistical comparisons of LEN dose-normalized PK parameter estimates for dose proportionality are provided in Table 7 for Day 1 (Week 1) and Day 36 (Week 6). The results of the ANOVA and power model analyses are in agreement, indicating close to dose proportional increase in LEN exposures between 200 mg and 400 mg once-weekly doses. For Week 1, increase in LEN Cₘₐₓ was close to dose proportional and C₁₆₈ₕ and AUC₀₋₁₆₈ₕ were slightly less than dose proportional. For Week 6, increase in Cₘₐₓ was dose proportional whereas C₁₆₈ₕ and AUC₀₋₁₆₈ₕ increase was less than dose proportional. However, the 90% CI ranged from 42.1% to 183% for Week 1 exposure parameters and 38.9% to 200% for Week 6 exposure parameters.

TABLE 7
Statistical Comparisons of Dose-Normalized LEN PK Parameters
for Dose Proportionality (MAD Cohorts, PK Analysis Set)

| | | | | | Statistical |
|---|---|---|---|---|---|
| | Treatment | | | | Comparison |
| | Test | | Reference | | % GLSM Ratio |
| PK Parameter | n | GLSM | n | GLSM | (90% CI) |

Compound 1 400 mg QW (Test) vs Compound 1 200 mg QW (Reference), Day 1

| | | | | | |
|---|---|---|---|---|---|
| Cₘₐₓ/D (ng/mL) | 9 | 22.3 | 9 | 24.7 | 90.3 (44.5, 183) |
| C_{168 h}/D (ng/mL) | 9 | 10.6 | 8 | 14.5 | 72.8 (43.4, 122) |
| AUC_{0-168 h}/D | 9 | 2400 | 8 | 3150 | 76.4 (42.1, 139) |
| (h · ng/mL) | | | | | |

Compound 1 400 mg QW (Test) vs Compound 1 200 mg QW (Reference), Day 36

| | | | | | |
|---|---|---|---|---|---|
| Cₘₐₓ/D (ng/mL) | 9 | 149 | 8 | 153 | 97.2 (47.4, 200) |
| C_{168 h}/D (h · ng/mL) | 9 | 30.2 | 8 | 51.7 | 58.4 (39.4, 86.7) |
| AUCₜₐᵤ/D (h · ng/mL) | 9 | 8940 | 8 | 14,100 | 63.2 (38.9, 103) |

ANOVA = analysis of variance; CI = confidence interval; GLSM = geometric least-squares mean; LEN = lenacapavir; MAD = multiple ascending dose; PK = pharmacokinetic(s); QW = once weekly; Cₜₐᵤ = C_{168 h} = C_{trough}; AUCₜₐᵤ = AUC_{0-168 h}.
The ANOVA model includes dose as a fixed effect.

### Example 8. Effect of Food on the Pharmacokinetics of Compound 1

The effect of food on Compound 1 was investigated in 2 cohorts. Participants in Cohort 11 (N=10) received a single dose of Compound 1 400 mg with a high-fat meal, and participants in Cohort 12 (N=10) received a single dose of Compound 1 1800 mg with a low-fat meal. Cohort 11 was compared against Week 1 data from MAD Cohort 7 with Compound 1 400 mg in fasted conditions since a single-dose cohort of 400 mg in fasted conditions was not conducted in the study.

### Effect of Low-Fat MEAL on Compound 1 1800 mg

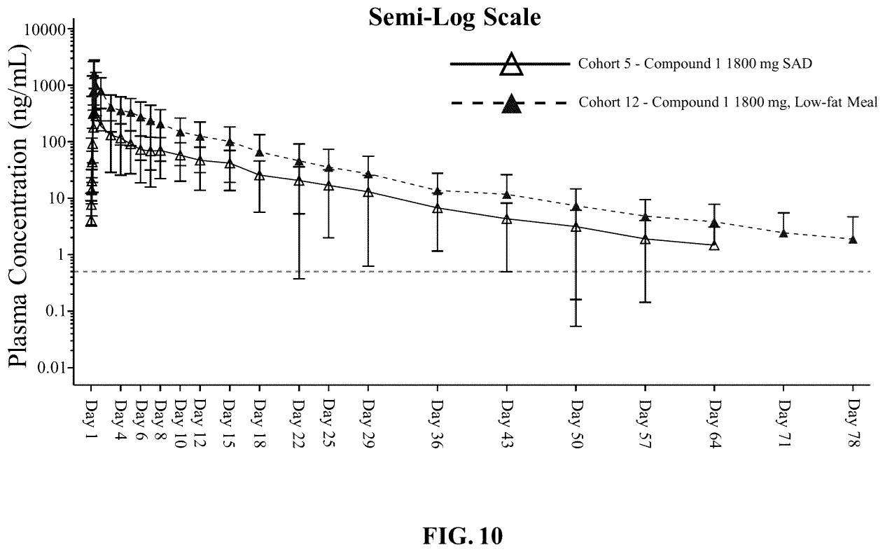

Administration of Compound 1 1800 mg with a low-fat meal increased LEN Cₘₐₓ by 8.87-fold and AUC_{inf} by 3.02-fold compared with fasting conditions (FIG. 10, Table 8). Median Tₘₐₓ was 8.00 hours with a low-fat meal compared with 12.0 hours under fasting conditions.

TABLE 8
Plasma PK Parameters and Comparisons of LEN Following Single Doses of Compound
1 1800 mg With Low-fat Meal Versus Fasted (FE Cohorts, PK Analysis Sets)

| | | | Cohort 12 | | |
|---|---|---|---|---|---|
| | Cohort 5 | | 1800 mg Fed (Low- | | % GLSM Ratio |
| Analyte/ PK | 1800 mg Fasted | | fat) | | (90% CI) |
| Parameter | (N = 8) | | (N = 10) | | Fed vs Fasted |
| Cₘₐₓ (ng/mL) | 168 | (154) | 1490 | (76.6) | 887 (425, 1850) |
| AUCₗₐₛₜ | 33,200 | (62.6) | 100,000 | (67.3) | 302 (185, 495) |
| (h · ng/mL) | | | | | |
| AUC_{inf} (h · ng/mL) | 33,500 | (62.4) | 101,000 | (67.4) | 302 (184, 494) |
| Tₘₐₓ (h) | 12.0 | (8.00, 18.0) | 8.00 | (6.00, 8.00) | — |
| t_{1/2} (h) | 241 | (209, 267) | 285 | (268, 298) | — |

CI = confidence interval; FE = food effect; % GCV = percentage geometric coefficient of variation; GLSM = geometric least-squares mean; LEN = lenacapavir; PK = pharmacokinetic(s); Q1 = first quartile; Q3 = third quartile.
All data are presented as geometric mean (% GCV) except time parameters, which are presented as median (Q1, Q3).

### Effect of High-Fat Meal on Compound 1400 mg

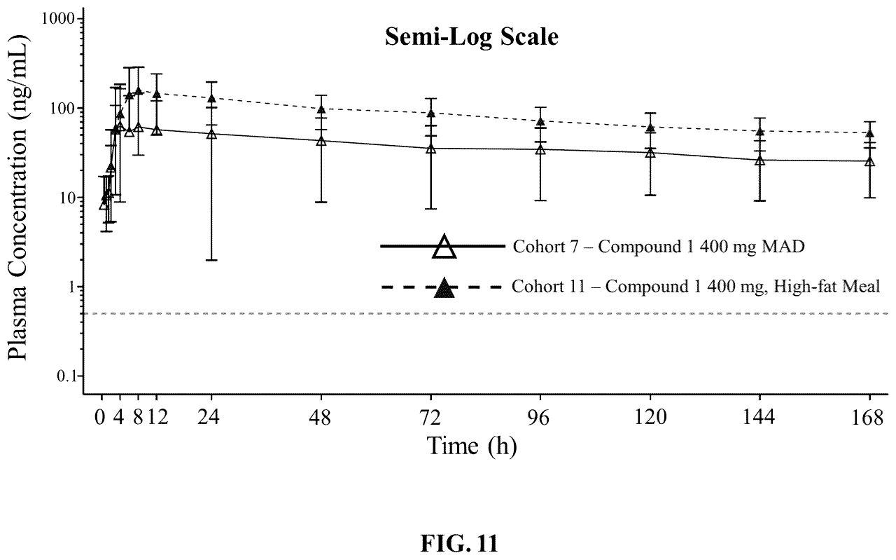

Administration of Compound 1 400 mg with a high-fat meal increased LEN Cₘₐₓ by 3.27-fold, C₁₆₈ₕ by 2.41-fold, and AUC₀₋₁₆₈ₕ by 2.69-fold compared with fasting conditions (FIG. 11, Table 9). Median Tₘₐₓ was 6.00 hours with a high-fat meal compared with 12.0 hours under fasting conditions.

TABLE 9
Plasma PK Parameters and Comparisons of LEN Following Single
Doses of Compound 1 400 mg With High-fat Meal Versus Fasted
(FE Cohort 11 vs MAD Cohort 7 Week 1, PK Analysis Sets)

| | Cohort 7 | | Cohort 11 | | % GLSM Ratio |
|---|---|---|---|---|---|
| Analyte/ PK | 400 mg (Fasted), | | 400 mg Fed (Low-fat) | | (90% CI) |
| Parameter | Week 1 (N = 9) | | (N = 10) | | Fed vs Fasted |
| Cₘₐₓ (ng/mL) | 44.7 | (141) | 146 | (79.9) | 327 (162, 661) |
| C_{168 h} (ng/mL) | 21.1 | (63.3) | 50.8 | (30.9) | 241 (167, 346) |
| AUC_{0-168 h} | 4810 | (85.1) | 12,900 | (45.2) | 269 (167, 432) |
| (h · ng/mL) | | | | | |
| Tₘₐₓ (h) | 12.0 | (4.00, 24.1) | 6.00 | (6.00, 12.0) | — |

CI = confidence interval; FE = food effect; % GCV = percentage geometric coefficient of variation; GLSM = geometric least-squares mean; LEN = lenacapavir; MAD = multiple ascending dose; PK = pharmacokinetic(s); Q1 = first quartile; Q3 = third quartile; QW = once weekly.
All data are presented as geometric mean (% GCV) except time parameters, which are presented as median (Q1, Q3).
AUC_{0-168 h} from Cohort 7 (400 mg QW) Week 1 data was used for comparison due to lack of single-dose data for 400 mg.

### Example 12. Urine Pharmacokinetics of Compound 1 and Lenacapavir

Urine was collected up to 168 hours postdose following SAD doses on Day 1 (Cohorts 1, 2, 4, and 5) or following the last MAD dose on Day 36 (Cohorts 6 and 7).

Compound 1: Urine samples for all SAD and MAD cohorts at all time points were BLQ (<5 ng/mL) for Compound 1, which indicates negligible renal elimination of unchanged Compound 1.

Lenacapavir: The mole fraction of the dose excreted in urine as LEN was <1% following single doses of Compound 1 200 mg to 2400 mg (Table 10) and multiple once-weekly doses over 6 weeks (Table 11), indicating negligible renal elimination of LEN.

TABLE 10
Urine PK Parameters of LEN Following Single Doses
of Compound 1 (SAD Cohorts, PK Analysis Sets)

| | Cohort 1 | Cohort 2 | Cohort 4 | Cohort 5 |
|---|---|---|---|---|
| Analyte/ PK | 200 mg Fasted | 600 mg Fasted | 2400 mg | 1800 mg |
| Parameter | (N = 8) | (N = 8) | Fasted (N = 8) | Fasted (N = 8) |
| Aₑ (mg) | 0.0136 (39.9)^{a} | 0.395 (482)^{b} | 0.143 (295) | 0.118 (80.4) |
| Fₑ (%) | 0.00912 (39.9)^{a} | 0.00882 (482)^{b} | 0.00799 (295) | 0.00877 (80.4) |
| CLᵣ (L/h) | 0.00462 (17.9)^{a} | 0.00504 (221)^{b} | 0.00977 (133) | 0.00778 (92.6) |

% GCV = percentage geometric coefficient of variation; LEN = lenacapavir; PK = pharmacokinetic(s); SAD = single ascending dose; ^{a}N = 2; ^{b}N = 7.
All data are presented as geometric mean (% GCV)

TABLE 11
Urine PK Parameters of LEN Following Multiple Once-Weekly
Doses of Compound 1 (MAD Cohorts, PK Analysis Sets)

| | Cohort 6 | Cohort 7 |
|---|---|---|
| | 200 mg QW Fasted | 400 mg QW Fasted |
| Analyte/ PK Parameter | Day 36 (N = 8) | Day 36 (N = 9) |
| Aₑ (mg) | 0.206 (97.0) | 0.135 (106) |
| Fₑ (%) | 0.138 (97.0) | 0.0451 (106) |
| CLᵣ (L/h) | 0.0145 (93.3) | 0.00753 (95.9) |

% GCV = percentage geometric coefficient of variation; LEN = lenacapavir; MAD = multiple ascending dose; PK = pharmacokinetic(s); QW = once weekly; ^{a} N = 6.
All data are presented as geometric mean (% GCV)

### Example 13. Safety Evaluation

A summary of safety results from this study is as follows:
Single and multiple doses of Compound 1 (up to 2400 mg single dose and 400 mg once weekly for 6 weeks) were safe and well tolerated when administered in the fasted or fed state.
The safety profile for Compound 1 did not differ with increasing doses of Compound 1, whether administered as a single or multiple doses, with or without food.
There were no Grade 3 or higher AEs, SAEs, deaths, or AEs leading to premature discontinuation of study drug or study.
The majority of laboratory abnormalities were Grade 1 or Grade 2 in severity.
No clinically significant changes in vital signs or safety ECG results were observed during the study.

### Example 14. Additional Tablet Formulations

Drug loading (DL) compositions of 40 w/w % and 60 w/w % of Compound 1 were evaluated for Phase 1 formulation development. The ingredients and composition of the 40 w/w % and 60 w/w % tablets are provided in Table 12.

TABLE 12
Tablets Containing 40 w/w % and 60 w/w % of Compound 1

| | Composition | | Composition | | | |
|---|---|---|---|---|---|---|
| | | mg/tablet | | mg/tablet | Quality | |
| Ingredient | w/w % | 55 mg | w/w % | 55 mg | Standard | Function |

Tablet Core

| | | | | | | |
|---|---|---|---|---|---|---|
| Compound 1 | 40.00 | 55.0 | 60.00 | 55.0 | In-house | Active |
| | | | | | | Ingredient |
| Mannitol | 27.25 | 37.47 | 17.25 | 15.81 | USP or Ph. | Diluent |
| | | | | | Eur. | |
| Microcrystalline | 27.25 | 37.47 | 17.25 | 15.81 | NF or Ph. | Diluent |
| Cellulose | | | | | Eur. | |
| Crospovidone | 4.00 | 5.5 | 4.00 | 3.67 | NF or Ph. | Disintegrant |
| | | | | | Eur. | |
| Magnesium | 1.5 | 2.06 | 1.50 | 1.38 | NF or Ph. | Lubricant |
| Stearate | | | | | Eur. | |
| Tablet Core | 100 | 137.5 | 100.0 | 91.7 | — | — |
| Total | | | | | | |

The bioavailability of the DL compositions with 40 w/w % and 60 w/w % of Compound 1 were evaluated in dogs. Tablets containing 55 mg of Compound 1 (equivalent to 40 mg LEN) with 40 w/w % and 60 w/w % drug loading were dosed in fasted, pentagastrin pre-treated dogs. Each formulation was dosed in 3 dogs in a cross-over study, where the same dogs were first dosed with the 40 w/w % DL tablets and then dosed two weeks later with the 60 w/w % DL tablets. The pharmacokinetics of LEN were measured over a one-week period. As shown in Table 13, the results demonstrated that drug loading did not have a significant effect on bioavailability.

TABLE 13
PK Profiles of LEN in Dogs Administered 40 w/w
% and 60 w/w % DL Compositions of Compound 1

| | Mean | | |
|---|---|---|---|
| | AUC₍₀₋ₗₐₛₜ₎ ± SD | Mean Cₘₐₓ ± | |
| Formulation | (nM · h) | SD (nM) | % F |
| Non-Precipitating | 13,040 ± 1,761 | 884 ± 244 | 18.1 ± 0.4 |
| Solution^{a} | | | |
| 40% DL Tablets^{b} | 9,599 ± 2,314 | 528 ± 133 | 13.0 ± 1.5 |
| 60% DL Tablets^{c} | 7,936 ± 1433 | 379 ± 170 | 10.7 ± 1.6 |
| LEN SDD Tablets^{d} | 2,822 ± 511 | 121 ± 49 | 4.6 ± 1 |

^{a}Non-Precipitating Solution: 5% ethanol, 20% propylene glycol (PG), 75% water, pH 7.33
^{b}40% DL Tablets: 40.4% Compound 1, 27.3% microcrystalline cellulose (MCC) (Avicel PH-101), 27.3% mannitol (200SD), 4.0 crospovidone (Polyplasdone XL), 1% magnesium stearate (d.90: 10 μm)
^{c}60% DL Tablets: 60.6% Compound 1, 17.2% MCC (Avicel PH-101), 17.2% mannitol (200SD), 4.0% crospovidone (Polyplasdone XL), 1% magnesium stearate (d.90: 10 μm)
^{d}LEN SDD Tablets: 20% SDD API, 5.34% copovidone, 8% croscarmellose sodium, 32.16% lactose, 1% magnesium stearate, 32.16% MCC, 1.34% Poloxamer 407

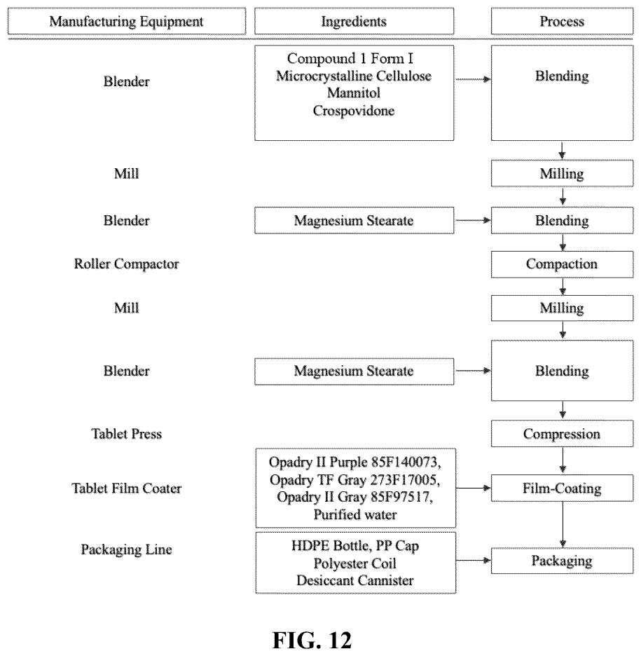

FIG. 12 shows a manufacturing process flow diagram for the preparation of 200 mg QW Compound 1 tablets and 1200 mg QM Compound 1 tablets. The composition of tablets with 200 mg of Compound 1 at 40 w/w/% drug loading, developed for QW Phase 3 PrEP, is provided in Table 14. The composition of tablets with 1200 mg of Compound 1 with 75 w/w % DL developed for QM dosing is provided in Table 15.

TABLE 14
Composition of Tablets with 200
mg of Compound 1 with 40 w/w % DL

| | Composition | | | |
|---|---|---|---|---|
| | | mg/tablet | Quality | |
| Ingredient | w/w % | 200 mg | Standard | Function |

Tablet Core

| | | | | |
|---|---|---|---|---|
| Compound 1 | 40.00 | 200.00 | In-house | Active |
| | | | | Ingredient |
| Mannitol | 27.25 | 136.25 | USP or Ph. | Diluent |
| | | | Eur. | |
| Microcrystalline | 27.25 | 136.25 | NF or Ph. Eur. | Diluent |
| Cellulose | | | | |
| Crospovidone | 4.00 | 20.00 | NF or Ph. Eur. | Disintegrant |
| Magnesium Stearate | 1.50 | 7.50 | NF or Ph. Eur. | Lubricant |
| Tablet Core Total | 100.0 | 500.0 | — | — |

Film-Coat

| | | | | |
|---|---|---|---|---|
| Opadry II Gray | 3.0 | 15.0 | In-house | Film-coat |
| 85F97517 | | | | |
| Opadry TF Gray | 3.0 | 15.0 | In-house | Film-coat |
| 273F17005 | | | | |
| Purified Water | — | TBD | USP or Ph. | Processing |
| | | | Eur. | Aid |

TABLE 15
Composition of Tablets with 1200
mg of Compound 1 with 75 w/w % DL

| | Composition | | | |
|---|---|---|---|---|
| | | mg/tablet | Quality | |
| Ingredient | w/w % | 1200 mg | Standard | Function |

Tablet Core

| | | | | |
|---|---|---|---|---|
| Compound 1^{a} | 75.00 | 1200.00 | In-house | Active |
| | | | | Ingredient |
| Mannitol^{a} | 7.75 | 124.00 | USP or Ph. | Diluent |
| | | | Eur. | |
| Microcrystalline | 7.75 | 124.00 | NF or Ph. Eur. | Diluent |
| Cellulose | | | | |
| Crospovidone | 8.00 | 128.00 | NF or Ph. Eur. | Disintegrant |
| Magnesium Stearate | 1.50 | 24.00 | NF or Ph. Eur. | Lubricant |
| Tablet Core Total | 100.0 | 1600 | — | — |

Film-Coat

| | | | | |
|---|---|---|---|---|
| Opadry II Purple | 3.0^{c} | 48.0 | In-house | Film-coat |
| 85F140073^{b} | | | | |
| Purified Water^{d} | — | TBD | USP or Ph. | Processing |
| | | | Eur. | Aid |

Tablets containing 100 mg and 600 mg of Compound 1 (crystalline Form I) with 40 w/w % or 75 w/w % drug loading (DL) were dosed in fasted, pentagastrin pre-treated dogs. Each formulation was dosed in 6 dogs in a cross-over study, where the same dogs were first dosed with one of the formulations and then dosed with the next formulation two weeks later, until all formulations were dosed. The pharmacokinetics of LEN in the dogs over a one-week period are shown in Table 16. The results demonstrated that the 75 w/w % tablet had a comparable PK profile to the Phase 1 tablets with 40 w/w % and the drug loading did not have a significant effect on bioavailability at the two different doses examined.

TABLE 16
PK Profiles of LEN in Dogs Administered 40 w/w %
and 75 w/w % DL Compositions of Compound 1

| | | API | | | | |
|---|---|---|---|---|---|---|
| | | Particle | Dose | Mean | Mean | |
| | | Size | (mg- | AUC₍₀₋ₗₐₛₜ₎ ± | Cₘₐₓ ± | |
| API | Formulation¹ | (d90 um) | fixed)¹ | SD (nM*h) | SD (nM)¹ | % F |
| Compound | Non- | — | 55 | 12,529 ± 2,111 | 873 ± 187 | 17.2 ± 3.5 |
| 1 | Precipitating | | | | | |
| | Solution^{2, 4} | | | | | |
| | Phase 1 | 81 | 100 | 16,949 ± 6,039 | 1,403 ± 604 | 14.5 ± 5.9 |
| | Formulation, | | | | | |
| | 40% DL³⁻⁴ | | | | | |
| | 75% DL, 8% | 16 | 100 | 21,143 ± 12,330 | 826 ± 211 | 16.4 ± 9.3 |
| | Disintegrant^{4, 6} | | | | | |
| | Phase 1 | 81 | 600 | 56,438 ± 43,277 | 2,180 ± 577 | 7.2 ± 5.4 |
| | Formulation, | | | | | |
| | 40% DL^{3-4, 6} | | | | | |
| | 75% DL, 8% | 16 | 600 | 46,171 ± 35,310 | 2,063 ± 554 | 6.0 ± 4.4 |
| | Disintegrant^{4, 6} | | | | | |

¹A 55 mg, 100 mg, and 600 mg-fixed dose of Compound 1 is equivalent to a 40 mg, 75 mg, and 450 mg-fixed dose of LEN, respectively.
²Non-Precipitating Solutions: 5% ethanol, 20% PG, 75% water, pH 7.33
³Dry Granulated Tablets 40 w/w % DL: 40.4% Compound 1, 27.3% MCC, 27.3% mannitol, 4.0 crospovidone, 1.5% magnesium stearate
⁴N = 6 for tablets. Non-precipitating solutions is a combination of two studies with 3 animals each.
⁵Dry Granulated Tablets 75 w/w % DL: 75% Compound 1, 7.75% MCC, 7.75% mannitol, 8.0 crospovidone, 1.5% magnesium stearate
⁶Manually compressed tablets.

Tablets with 100 mg of Compound 1 with 40 w/w/% DL and a range of drug substance particle sizes (d90 of 16 μm to 187 μm) were dosed in fasted, pentagastrin pre-treated dogs. Each formulation was dosed in 6 dogs in a cross-over study, where the same dogs were first dosed with one of the formulations and then dosed with the next formulation two weeks later, until all formulations were dosed. The pharmacokinetics of LEN in the dogs over a one-week period are shown in Table 17. The results demonstrated that the drug substance particle size did not have a significant effect on bioavailability.

TABLE 17
PK Profiles of LEN in Dogs Administered Tablets with 100
mg of Compound 1 with Different API Particle Sizes

| | | API | | | | |
|---|---|---|---|---|---|---|
| | | Particle | Dose | Mean | Mean | |
| | | Size | (mg- | AUC₍₀₋ₗₐₛₜ₎ ± | Cₘₐₓ ± | |
| API | Formulation¹ | (d90 um) | fixed)¹ | SD (nM*h) | SD (nM)¹ | % F |
| Compound | Non- | — | 55 | 12,529 ± 2,111 | 873 ± 187 | 17.2 ± 3.5 |
| 1 | Precipitating | | | | | |
| | Solution^{2, 5} | | | | | |
| | Dry | 16 | 100 | 23,406 ± 8,114 | 1,719 ± 643 | 19.9 ± 7.1 |
| | Granulated | 22 | | 13,125 ± 7,084 | 661 ± 131 | 10.2 ± 5.5 |
| | Tablet^{3, 5-6} | 85 | | 16,949 ± 6,039 | 1,403 ± 604 | 14.5 ± 5.9 |
| | Phase I | 136 | | 16,423 ± 2,124 | 915 ± 309 | 13.2 ± 1.6 |
| | Formulation, | 187 | | 13,860 ± 4,976 | 1,053 ± 501 | 11.7 ± 4.3 |
| | 40% DL | | | | | |
| LEN | SDD Tablets^{4, 5} | — | 40 | 2,822 ± 511 | 121 ±4 9 | 4.6 ± 1 |

¹A 55 mg-fixed dose of Compound 1 is equivalent to a 40 mg-fixed dose of LEN. A 100 mg-fixed dose of Compound 1 is equivalent to a 75 mg-fixed dose of LEN.
²Non-Precipitating Solutions: 5% Ethanol, 20% PG, 75% Water, pH 7.33
³Dry Granulated Tablets: 40.4% Compound 1, 27.3% MCC, 27.3% Mannitol, 4.0 Crospovidone, 1% Magnesium Stearate.
⁴LEN SDD Tablets: 20% SDD API, 5.34% Copovidone, 8% Croscamellose Sodium, 32.16% Lactose, 1% magnesium stearate, 32.16% Microcrystalline cellulose, 1.34% Poloxamer 407.
⁵N = 3 for 55 mg manually compressed tablets. N = 6 for manufactured 100 mg tablets and LEN SDD tablets. Non-precipitating solutions were a combination of two studies with 3 animals each.
⁶Data not significantly different based on student t-test (p-values > 0.15)

Compound 1 100 mg tablets packaged in a 45 mL bottle in a 10-count configuration were placed at 40° C., 75% relative humidity (RH) and 30° C., 75% RH conditions. At each timepoint, tablets were assessed for appearance, water content, assay and impurities/degradants, dissolution, and microbial content (Table 18). Minimal degradation (0.5%) was observed at accelerated conditions of 40° C., 75% RH at six months and at ambient conditions of 30° C., 75% RH at 24 months.

TABLE 18
Stability of 100 mg Compound 1 Tablets (GMP, Phase 1)

| | | | | 40° C./75% RH | | | | | | 30° C./75% RH | |
|---|---|---|---|---|---|---|---|---|---|---|---|
| Test Attributes | | Initial | | 1 M | | 3 M | | 6 M | | 3 M | |
| Appearance | | Conforms | | Conforms | | Conforms | | Conforms | | Conforms | |
| Water Content (%) | | 2.0 | | 1.0 | | 1.1 | | 1.6 | | 0.91 | |
| Assay (%) | | 100.9 | | 98.9 | | 98.9 | | 98.7 | | 100.8 | |
| Impurity/Deg | Total | 0.1 | | 0.2 | | 0.3 | | 0.5 | | 0.1 | |
| Content (%) | LEN | — | | 0.06 | | 0.1 | | 0.17 | | Tr (0.05) | |
| | Time (min) | Avg | SD | Avg | SD | Avg | SD | Avg | SD | Avg | SD |
| Dissolution | 10 | 90 | 1.9 | 86 | 2 | 85 | 2.6 | 87 | 1.7 | 84 | 2.7 |
| | 15 | 97 | 1.4 | 98 | 0.8 | 96 | 1.6 | 98 | 0.9 | 98 | 0.3 |
| | 20 | 98 | 1.8 | 100 | 1.1 | 98 | 2.2 | 100 | 1.5 | 100 | 0.8 |
| | 30 | 98 | 1.6 | 100 | 1 | 98 | 2.5 | 101 | 1.6 | 101 | 0.9 |
| | 45 | 99 | 1.7 | 100 | 1.1 | 98 | 2.2 | 100 | 1.1 | 101 | 0.7 |
| | 60 | 99 | 1.8 | 100 | 1.1 | 99 | 2.3 | 100 | 1.8 | 101 | 0.8 |

| | | | | | | |
|---|---|---|---|---|---|---|
| Microbiological | TAMC | <100 | NT | NT | NT | NT |
| Examination | TYMC | <50 | NT | NT | NT | NT |
| (CFU/g) | E. coli | Absent | NT | NT | NT | NT |

| | | 30° C./75% RH | | | | | | | | | |
|---|---|---|---|---|---|---|---|---|---|---|---|
| Test Attributes | | 6 M | | 9 M | | 12 M | | 18 M | | 24 M | |
| Appearance | | Conforms | | Conforms | | Conforms | | Conforms | | Conforms | |
| Water Content (%) | | 1.2 | | 1.3 | | 1.4 | | 1.7 | | 1.8 | |
| Assay (%) | | 99.9 | | 99.3 | | 99.4 | | 99.7 | | 98.7 | |
| Impurity/Deg | Total | 0.3 | | 0.3 | | 0.3 | | 0.5 | | 0.5 | |
| Content (%) | LEN | 0.08 | | 0.08 | | 0.10 | | 0.15 | | 0.18 | |
| | Time (min) | Avg | SD | Avg | SD | Avg | SD | Avg | SD | Avg | SD |
| Dissolution | 10 | 87 | 2.9 | 86 | 5.8 | 85 | 4.1 | 88 | 3.3 | 86 | 1.9 |
| | 15 | 97 | 3.6 | 98 | 2.5 | 99 | 2.4 | 97 | 2.0 | 96 | 2.0 |
| | 20 | 99 | 3.5 | 100 | 3.1 | 102 | 2.9 | 98 | 2.5 | 98 | 2.1 |
| | 30 | 99 | 3.4 | 100 | 3.1 | 101 | 2.9 | 98 | 2.3 | 99 | 1.8 |
| | 45 | 99 | 3.5 | 100 | 3.1 | 103 | 2.7 | 98 | 2.0 | 99 | 1.8 |
| | 60 | 99 | 3.7 | 100 | 3.3 | 103 | 3.1 | 99 | 2.2 | 99 | 1.8 |

| | | | | | | |
|---|---|---|---|---|---|---|
| Microbiological | TAMC | NT | NT | <100 | NT | <100 |
| Examination | TYMC | NT | NT | <50 | NT | <50 |
| (CFU/g) | E. coli | NT | NT | Absent | NT | Absent |

Conforms (Appearance): Compound 1 Tablets, 100 mg, are round, plain-faced, film-coated purplish-pink tablets. Tr: trace (≥0.025% and <0.05%) Packaging: 10 tablets/bottle, 45 mL, white HDPE bottle with 1 g desiccant and polyester coil.

Compound 1, 400 mg tablets packaged in a 45 mL bottle in a 10-count configuration were placed at 40 CC, 75% relative humidity (RH) and 30° C., 75% RH conditions. At each timepoint, tablets were assessed for appearance, water content, assay and impurities/degradants, dissolution, and microbial content (Table 19). Minimal degradation (0.3-0.4%) was observed at accelerated conditions of 40° C., 75% RH at six months and at ambient conditions of 30° C., 75% RH at 24 months.

TABLE 19
Stability of 400 mg Compound 1 Tablets (GMP, Phase 1)

| | | | | 50° C. | | −20° C. | | 40° C./75% RH | | | | | |
|---|---|---|---|---|---|---|---|---|---|---|---|---|---|
| Test Attributes | | Initial | | 2 WK | | 1 M | | 1 M | | 3 M | | 6 M | |
| Appearance | | Conforms | | Conforms | | Conforms | | Conforms | | Conforms | | Conforms | |
| Water Content (%) | | 1.5 | | 0.72 | | 1.1 | | 0.76 | | 0.79 | | 0.92 | |
| Assay (%) | | 100.2 | | 99.9 | | 99.3 | | 100.2 | | 100 | | 100.5 | |
| Impurity/Deg | Total | 0.1 | | 0.2 | | 0.1 | | 0.1 | | 0.2 | | 0.3 | |
| Content (%) | LEN | — | | 0.05 | | — | | Tr (0.05) | | 0.08 | | 0.11 | |
| | Time (min) | Avg | SD | Avg | SD | Avg | SD | Avg | SD | Avg | SD | Avg | SD |
| Dissolution | 10 | 84 | 2.4 | 78 | 5.2 | 85 | 2.4 | 84 | 6.2 | 77 | 6.7 | 80 | 5.2 |
| | 15 | 96 | 1.8 | 95 | 0.9 | 95 | 1.2 | 95 | 2.2 | 94 | 0.5 | 94 | 1.5 |
| | 20 | 98 | 2.9 | 100 | 1.4 | 97 | 1.6 | 96 | 3 | 98 | 2.2 | 97 | 1.7 |
| | 30 | 100 | 2.5 | 101 | 1.7 | 98 | 1.7 | 97 | 3 | 99 | 3 | 99 | 1.3 |
| | 45 | 100 | 2.9 | 101 | 1.6 | 98 | 1.7 | 98 | 3 | 100 | 3 | 100 | 1.4 |
| | 60 | 100 | 3.1 | 102 | 1.6 | 98 | 1.7 | 98 | 3.1 | 100 | 2.9 | 99 | 1.2 |

| | | | | | | | |
|---|---|---|---|---|---|---|---|
| Microbiological | TAMC | <100 | N/A | N/A | N/A | N/A | N/A |
| Examination | TYMC | <50 | N/A | N/A | N/A | N/A | N/A |
| (CFU/g) | E. coli | Absent | N/A | N/A | N/A | N/A | N/A |

| | | 30 C./75% RH | | | | | | | | | | | |
|---|---|---|---|---|---|---|---|---|---|---|---|---|---|
| Test Attributes | | 3 M | | 6 M | | 9 M | | 12 M | | 18 M | | 24 M | |
| Appearance | | Conforms | | Conforms | | Conforms | | Conforms | | Conforms | | Conforms | |
| Water Content (%) | | 0.7 | | 1.0 | | 0.92 | | 0.92 | | 1.2 | | 1.1 | |
| Assay (%) | | 100.9 | | 100.9 | | 99.0 | | 100.7 | | 100.3 | | 102.0 | |
| Impurity/Deg | Total | 0.1 | | 0.2 | | 0.3 | | 0.2 | | 0.3 | | 0.4 | |
| Content (%) | LEN | Tr (0.05) | | 0.06 | | 0.1 | | 0.1 | | 0.10 | | 0.10 | |
| | Time (min) | Avg | SD | Avg | SD | Avg | SD | Avg | SD | Avg | SD | Avg | SD |
| Dissolution | 10 | 78 | 5.6 | 81 | 3.3 | 82 | 3.8 | 80 | 4.5 | 84 | 3.3 | 86 | 2.2 |
| | 15 | 94 | 1.2 | 94 | 1.9 | 95 | 0.6 | 94 | 0.9 | 96 | 1.0 | 95 | 0.8 |
| | 20 | 98 | 1.3 | 97 | 2.1 | 99 | 1.1 | 98 | 1.7 | 99 | 1.8 | 97 | 1.0 |
| | 30 | 99 | 1.5 | 98 | 2.3 | 100 | 1.1 | 98 | 1.3 | 99 | 2.1 | 98 | 1.1 |
| | 45 | 100 | 1.6 | 99 | 2.2 | 100 | 1.3 | 101 | 1.8 | 100 | 1.6 | 98 | 1.2 |
| | 60 | 100 | 1.7 | 99 | 2.1 | 100 | 1.1 | 101 | 1.8 | 100 | 1.7 | 99 | 1.0 |

| | | | | | | | |
|---|---|---|---|---|---|---|---|
| Microbiological | TAMC | N/A | N/A | N/A | <100 | N/A | <100 |
| Examination | TYMC | N/A | N/A | N/A | <50 | N/A | <50 |
| (CFU/g) | E. coli | N/A | N/A | N/A | Absent | N/A | Absent |

Conforms (Appearance): Compound 1 Tablets, 400 mg, are capsule-shaped, plain-faced, film-coated purplish-pink tablets. Tr: trace (≥0.025% and <0.05%). Packaging: 10 tablets/bottle, 45 mL, white HDPE bottle with 3 g desiccant and polyester coil

Compound 1 300 mg tablets packaged in a 45 mL bottle in a 4-count configuration were placed at 40° C., 75% RH and 30° C., 75% RH conditions. At each timepoint, tablets were assessed for appearance, water content, assay and impurities/degradants, dissolution, and microbial content (Table 20). Minimal degradation (0.2-0.3%) was observed at accelerated conditions of 40° C., 75% RH at six months and at ambient conditions of 30° C., 75% RH at 12 months.

TABLE 20
Stability of 300 mg Compound 1 Tablets (GMP, Phase 2)

| | | | | 40° C./75% RH | | | | | |
|---|---|---|---|---|---|---|---|---|---|
| Test Attributes | | Initial | | 1 M | | 3 M | | 6 M | |
| Appearance | | Conforms | | Conforms | | Conforms | | Conforms | |
| Water Content (%) | | 0.92 | | 0.81 | | 0.78 | | 1.0 | |
| Assay (%) | | 100.9 | | 100.7 | | 99.5 | | 100.1 | |
| Impurity/Deg | Total | 0.0 | | 0.1 | | 0.2 | | 0.2 | |
| Content (%) | LEN | — | | 0.08 | | 0.08 | | 0.11 | |
| | Time (min) | Avg | SD | Avg | SD | Avg | SD | Avg | SD |
| Dissolution | 10 | 91 | 4.0 | 89 | 4.2 | 91 | 1.8 | 90 | 4.4 |
| | 15 | 100 | 1.0 | 99 | 1.4 | 99 | 1.6 | 99 | 1.2 |
| | 20 | 101 | 1.6 | 101 | 1.8 | 101 | 1.9 | 100 | 1.6 |
| | 30 | 101 | 1.9 | 101 | 2.0 | 101 | 2.3 | 100 | 1.6 |
| | 45 | 101 | 1.5 | 101 | 1.9 | 101 | 1.7 | 100 | 1.8 |
| | 60 | 101 | 1.8 | 101 | 1.9 | 101 | 2.0 | 100 | 1.7 |
| Microbiological | TAMC | <100 | | N/A | | N/A | | N/A | |
| Examination | TYMC | <50 | | N/A | | N/A | | N/A | |
| (CFU/g) | E. coli | Absent | | N/A | | N/A | | N/A | |
| | | 30° C./75% RH | | | | | | | |
| Test Attributes | | 3 M | | 6 M | | 9 M | | 12 M | |
| Appearance | | Conforms | | Conforms | | Conforms | | Conforms | |
| Water Content (%) | | 0.66 | | 0.9 | | 0.91 | | 1.0 | |
| Assay (%) | | 100.1 | | 100.3 | | 100.1 | | 101.5 | |
| Impurity/Deg | Total | 0.1 | | 0.1 | | 0.2 | | 0.3 | |
| Content (%) | LEN | Tr (0.043%) | | 0.06 | | 0.08 | | 0.11 | |
| | Time (min) | Avg | SD | Avg | SD | Avg | SD | Avg | SD |
| Dissolution | 10 | 90 | 1.8 | 90 | 2.2 | 93 | 0.8 | 89 | 4.1 |
| | 15 | 99 | 0.6 | 98 | 1.0 | 100 | 1.0 | 100 | 0.9 |
| | 20 | 101 | 0.8 | 99 | 0.9 | 101 | 1.2 | 101 | 1.4 |
| | 30 | 101 | 1.3 | 99 | 1.1 | 101 | 0.8 | 101 | 1.6 |
| | 45 | 101 | 1.4 | 99 | 1.1 | 101 | 0.8 | 101 | 1.5 |
| | 60 | 101 | 0.8 | 99 | 1.2 | 101 | 0.8 | 101 | 1.4 |

| | | | | | |
|---|---|---|---|---|---|
| Microbiological | TAMC | N/A | N/A | N/A | <100 |
| Examination | TYMC | N/A | N/A | N/A | <50 |
| (CFU/g) | E. coli | N/A | N/A | N/A | Absent |

Conforms (Appearance): Compound 1 Tablets, 300 mg, are round, plain-faced, film-coated purplish-pink tablets. Tr: trace (≥0.025% and <0.05%). Packaging: 100 mg and 400 mg tablets: 10 tablets/bottle. 300 mg tablets: 4 tablets/bottle, 45 mL, white HDPE bottles with 1 g desiccant and polyester coil.

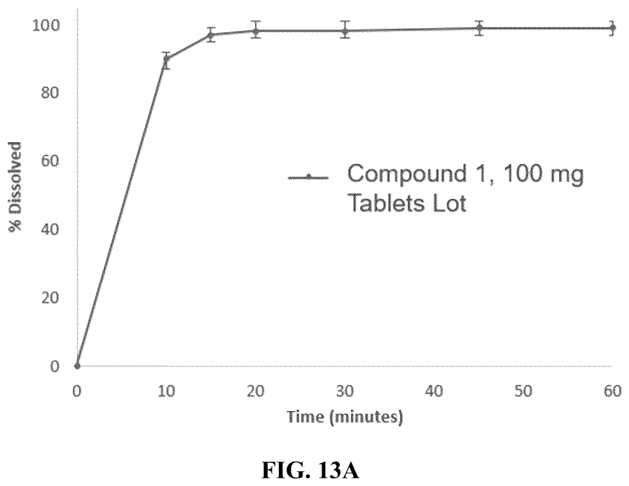

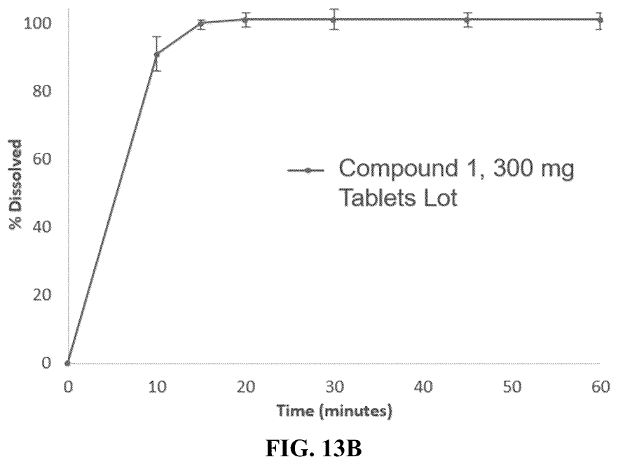

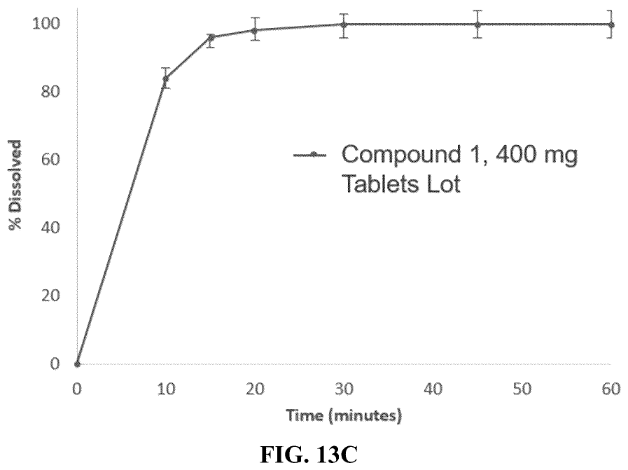

The dissolution of 100 mg, 300 mg, and 400 mg Compound 1 tablets were assessed in an appropriate dissolution media. Tablets were placed in 900 mL of media (composed of 50 mM acetate buffer at pH 4.9 with 0.5% SDS) with a USP/Ph. Eur. apparatus 2 paddle at 75 rpm. At 10 minute, 15 minute, 20 minute, 30 minute, 45 minute, and 60 minute time intervals, samples were taken from the media and analyzed for Compound 1 via UPLC method. The dissolution profiles are shown in FIGS. 13A-13C.

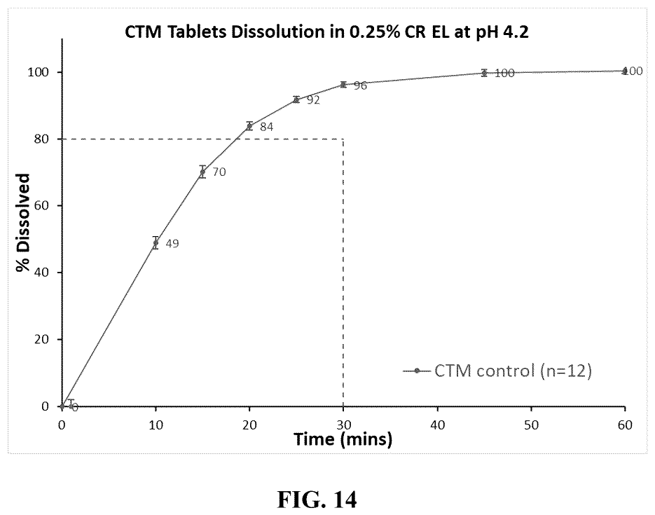

The dissolution of 200 mg Compound 1 tablets were assessed via a dissolution method. Tablets were placed in 900 mL of media composed of 125 mM sodium acetate buffer at pH 4.2 with a USP/Ph. Eur. apparatus 2 paddle at 75 rpm. At 10, 15, 20, 25, 30, 45, and 60 minute time intervals, samples were taken from the media and analyzed for Compound 1 via UPLC method. The dissolution profile is shown in FIG. 14.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A tablet comprising a compound of Formula Ia: 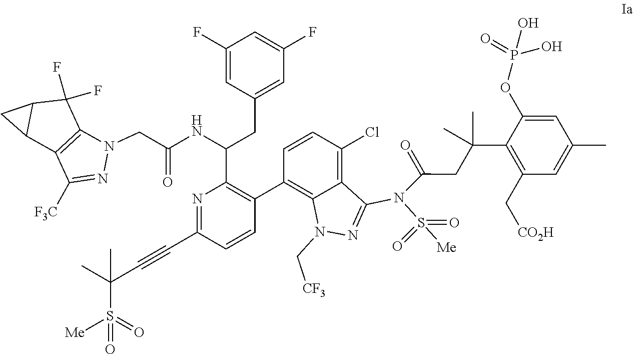 [SMILES: Cc1cc(CC(=O)O)c(C(C)(C)CC(=O)N(c2nn(CC(F)(F)F)c3c(-c4ccc(C#CC(C)(C)S(C)(=O)=O)nc4C(Cc4cc(F)cc(F)c4)NC(=O)Cn4nc(C(F)(F)F)c5c4C(F)(F)C4CC54)ccc(Cl)c23)S(C)(=O)=O)c(OP(=O)(O)O)c1]
or a pharmaceutically acceptable salt thereof, and one or more pharmaceutically acceptable excipients.

2. The tablet of claim 1, wherein the tablet comprises about 30 w/w % to about 50 w/w % of the compound of Formula Ia, or a pharmaceutically acceptable salt thereof.

3-4. (canceled)

5. The tablet of claim 1, wherein the tablet comprises about 25 w/w % to about 35 w/w % of mannitol.

6-7. (canceled)

8. The tablet of claim 1, wherein the tablet comprises about 25 w/w % to about 35 w/w % of microcrystalline cellulose.

9-10. (canceled)

11. The tablet of claim 1, wherein the tablet comprises about 1 w/w % to about 10 w/w % of crospovidone.

12-13. (canceled)

14. The tablet of claim 1, wherein the tablet comprises about 1 w/w % to about 5 w/w % of magnesium stearate.

15-16. (canceled)

17. The tablet of claim 1, wherein the tablet comprises:
about 30 w/w % to about 50 w/w % of the compound of Formula Ia, or a pharmaceutically acceptable salt thereof;
about 25 w/w % to about 35 w/w % of mannitol;
about 25 w/w % to about 35 w/w % of microcrystalline cellulose;
about 1 w/w % to about 10 w/w % of crospovidone; and
about 1 w/w % to about 5 w/w % of magnesium stearate.

18. (canceled)

19. The tablet of claim 1, wherein the tablet comprises:
about 40 w/w % of the compound of Formula Ia, or a pharmaceutically acceptable salt thereof,
about 27.25 w/w % of mannitol;
about 27.25 w/w % of microcrystalline cellulose;
about 4 w/w % of crospovidone; and
about 1.5 w/w % of magnesium stearate.

20. The tablet of claim 1, wherein the tablet comprises about 5 mg to about 500 mg of the compound of Formula Ia, or a pharmaceutically acceptable salt thereof.

21-24. (canceled)

25. The tablet of claim 1, wherein the tablet comprises about 50 mg to about 300 mg of mannitol.

26-27. (canceled)

28. The tablet of claim 1, wherein the tablet comprises about 50 mg to about 300 mg of microcrystalline cellulose.

29-30. (canceled)

31. The tablet of claim 1, wherein the tablet comprises about 5 mg to about 50 mg of crospovidone.

32-33. (canceled)

34. The tablet of claim 1, wherein the tablet comprises about 1 mg to about 20 mg of magnesium stearate.

35-36. (canceled)

37. The tablet of claim 1, wherein the tablet comprises:
about 5 mg to about 500 mg of the compound of Formula Ia, or a pharmaceutically acceptable salt thereof;
about 50 mg to about 300 mg of mannitol;
about 50 mg to about 300 mg of microcrystalline cellulose;
about 5 mg to about 50 mg of crospovidone; and
about 1 mg to about 20 mg of magnesium stearate.

38. (canceled)

39. The tablet of claim 1, wherein the tablet comprises:
about 200 mg of the compound of Formula Ia, or a pharmaceutically acceptable salt thereof,
about 136.25 mg of mannitol;
about 136.25 mg of microcrystalline cellulose;
about 20 mg of crospovidone; and
about 7.5 mg of magnesium stearate.

40. The tablet of claim 1, wherein the tablet further comprises an outer film coat.

41-43. (canceled)

44. The tablet of claim 1, wherein the compound of Formula Ia, or a pharmaceutically acceptable salt thereof, is a compound of Formula Ib: 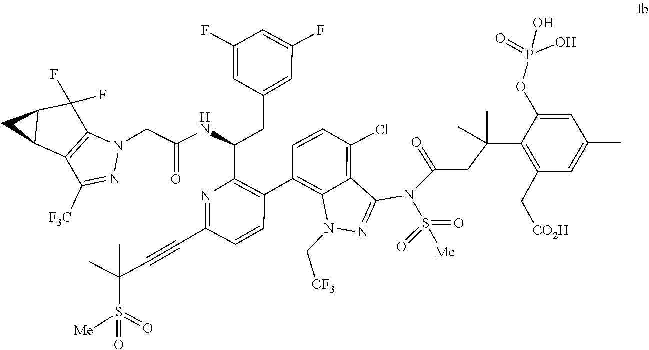 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]
or a pharmaceutically acceptable salt thereof.

45. The tablet of claim 1, wherein the compound of Formula Ia, or a pharmaceutically acceptable salt thereof, is a crystalline form of a compound of Formula Ib:

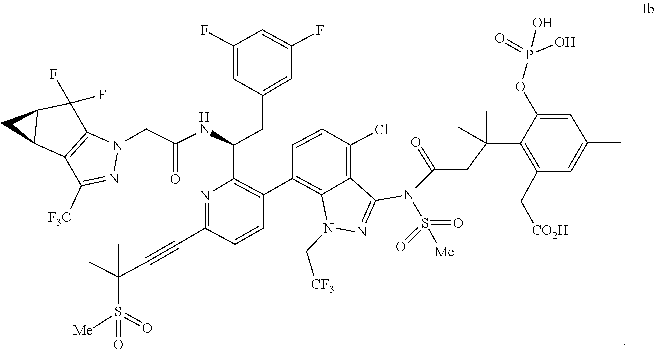 [SMILES: Cc1cc(CC(=O)O)c(C(C)(C)CC(=O)N(c2nn(CC(F)(F)F)c3c(-c4ccc(C#CC(C)(C)S(C)(=O)=O)nc4[C@H](Cc4cc(F)cc(F)c4)NC(=O)Cn4nc(C(F)(F)F)c5c4C(F)(F)[C@@H]4C[C@H]54)ccc(Cl)c23)S(C)(=O)=O)c(OP(=O)(O)O)c1]

46. (canceled)

47. A method of treating or preventing human immunodeficiency virus (HIV) infection in a patient, comprising administering to the patient a tablet of claim 1.

48-74. (canceled)

75. A method of reducing the risk of sexually acquired HIV in a patient, comprising administering to the patient a tablet of claim 1.

76-89. (canceled)
